SEQUENCE LISTING (6 sequences)

SEQ ID NO: 1 - DNA, 1356 bp, Cupriavidus necator
  source 1..1356: mol_type = unassigned DNA
ttagattgaacgctggcggcatgccttacacatgcaagtcgaacggcagcacgggcttcggcctggtggcgagtggcgaacgggtgagtaatacatcggaacgtgccctgtagtgggggataactagtcgaaagattagctaataccgcatacgacctgagggtgaaagcgggggaccgcaaggcctcgcgctacaggagcggccgatgtctgattagctagttggtggggtaaaagcctaccaaggcgacgatcagtagctggtctgagaggacgatcagccacactgggactgagacacggcccagactcctacgggaggcagcagtggggaattttggacaatgggggcaaccctgatccagcaatgccgcgtgtgtgaagaaggccttcgggttgtaaagcacttttgtccggaaagaaatggctctggttaatacccggggtcgatgacggtaccggaagaataagcaccggctaactacgtgccagcagccgcggtaatacgtagggtgcgagcgttaatcggaattactgggcgtaaagcgtgcgcaggcggttttgtaagacaggcgtgaaatccccgagctcaacttgggaatggcgcttgtgactgcaaggctagagtatgtcagaggggggtagaattccacgtgtagcagtgaaatgcgtagagatgtggaggaataccgatggcgaaggcagccccctgggacgtcactgacgctcatgcacgaaagcgtggggagcaaacaggattagataccctggtagtccacgccctaaacgatgtcaactagttgttggggattcatttcttcagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgccactaacgaagcagagatgcattaggtgcccgaaagggaaagtggacacaggtgctgcatggctgtcgtcagctcgtgtcgtgagatgttgggttaagtcccgcaacgagcgcaacccttgtctctagttgctacgaaagggcactctagagagactgccggtgacaaaccggaggaaggtggggatgacgtcaagtcctcatggcccttatgggtagggcttcacacgtcatacaatggtgcgtacagagggttgccaacccgcgagggggagctaatcccagaaaacgcatcgtagtccggatcgtagtctgcaactcgactacgtgaagctggaatcgctagtaatcgcggatcagcatgccgcggtgaatacgttcccggtct

SEQ ID NO: 2 - DNA, 1537 bp, Cupriavidus necator
  source 1..1537: mol_type = unassigned DNA
agattgaactgaagagtttgatcctggctcagattgaacgctggcggcatgccttacacatgcaagtcgaacggcagcacgggcttcggcctggtggcgagtggcgaacgggtgagtaatacatcggaacgtgccctgtagtgggggataactagtcgaaagattagctaataccgcatacgacctgagggtgaaagcgggggaccgcaaggcctcgcgctacaggagcggccgatgtctgattagctagttggtggggtaaaagcctaccaaggcgacgatcagtagctggtctgagaggacgatcagccacactgggactgagacacggcccagactcctacgggaggcagcagtggggaattttggacaatgggggcaaccctgatccagcaatgccgcgtgtgtgaagaaggccttcgggttgtaaagcacttttgtccggaaagaaatggctctggttaatacccggggtcgatgacggtaccggaagaataagcaccggctaactacgtgccagcagccgcggtaatacgtagggtgcgagcgttaatcggaattactgggcgtaaagcgtgcgcaggcggttttgtaagacaggcgtgaaatccccgagctcaacttgggaatggcgcttgtgactgcaaggctagagtatgtcagagggggaagaattccacgtgtagcagtgaaatgcgtagagatgtggaggaataccgatggcgaaggcagccccctgggacgtcactgacgctcatgcacgaaagcgtggggagcaaacaggattagataccctggtagtccacgccctaaacgatgtcaactagttgttggggattcatttcttcagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgccactaacgaagcagagatgcattaggtgcccgaaagggaaagtggacacaggtgctgcatggctgtcgtcagctcgtgtcgtgagatgttgggttaagtcccgcaacgagcgcaacccttgtctctagttgctacgaaagggcactctagagagactgccggtgacaaaccggaggaaggtggggatgacgtcaagtcctcatggcccttatgggtagggcttcacacgtcatacaatggtgcgtacagagggttgccaacccgcgagggggagctaatcccagaaaacgcatcgtagtccggatcgtagtctgcaactcgactacgtgaagctggaatcgctagtaatcgcggatcagcatgccgcggtgaatacgttcccgggtcttgtacacaccgcccgtcacaccatgggagtgggttttgccagaagtagttagcctaaccgcaaggagggcgattaccacggcagggttcatgactggggtgaagtcgtaacaaggtagccgtatcggaaggtgcggctggatcacctcctttc

SEQ ID NO: 3 - DNA, 209 bp, Cupriavidus necator
  source 1..209: mol_type = unassigned DNA
gcctcgctgctttccacctggcgccgcacgcggccccagacgtcgatttcccaggttgcgcccagggtcgcgctctgcccgttgagcgtgctgccgctggcgccgcgcgcgcgcgaggcgccggcctgtgcgtcgacggtcgggaagaagccggcgcgcgcggcctgcagcgacgccaccgcctggcggtactgcgcctcggcggcctt

SEQ ID NO: 4 - DNA, 1495 bp, Cupriavidus necator
  source 1..1495: mol_type = unassigned DNA
aggcgccggcctgtgcgtcgacggtcgggaagaagccggcgcgcgcggcctgcagcgacgccaccgcctggcggtactgcgcctcggcggccttgatgttctggttcgagatctgcacctcggacatcagcgcgtcgagctgcgcatcgccgaacacggtccaccagtcggcgcgtgccagcgcatcctgcggctcggcgggcttccagtcgccggtccaggcgggggtggcggcatcggcttccttgaaggatgcggaaaccggcgcgtcggggcgctggtagtcggggccgacggcgcagccggccagcagcagcgcgcaggccagcgacaccggcagggcatgggtcaggaggcgggaaagaactgtcatgtcgagtcttcgcaaatctagacggcggccggctggtcaggcgtgccggcaccacggcggcgctggcgccaggccttgaccttcaggcgccagcggtccagcgtcaggtagaccaccggcgtggtgtacagcgtcagcagctggcttaccaccagtccgccgacaatggagatgcccagcggcgcgcgcagttcggcgccgtcgccgcggccgattgccagcggcaccgcgcccagcagcgcggccatggtggtcatcaggatcgggcggaagcgcagcaggcaggcgcggtagatcgcgtcgcgcggcgacaggccatcgcgccgttcggcatcgatggcgaagtcgatcatcatgatcgcgttctttttcacgatgccgatcagcaggatcacgccgatcagcgcgatgatgctgaagtcggtcttcgatgccagcagcgccagcagcgcgcccacgccggcggagggcagcgtcgacaggatcgtcagcggatgcacatagctttcatacagcacgcccagcacgatgtagatcgtgatcagcgccgccaggatcaggatcggctgactcttgagcgaatcctggaacgccttggcgccgccctggaagttggcgcgcagcgtctccggcacgccgatgcgcgccatctcgcgcgtgatcgcgtcggtcgcctgcgacagcgaagtgccctcggccaggttgaacgagatcgtcgaggccgcgaactggccctggtggttcacgcccagcggcgtgctggacggggtcacgcgcgcgaacgccgccagcggcacgcggttgccgttgccggtgaccacgtagatgtccttgagcgcatcgggcccttgcaggtattcctggctcagctccatcaccacgcggtactggttcagcggatggtagatggtggacaccagccgctggccgaaggcatcgttgagcaccgcatccacctgctgcgcggtcacgcccaggcgcgaggccgcgtcgcggtcgatgatcaccgaggtctgcaggcccttgtcgttggtatcggtgtcgatatcctccagccccttcaggttcgacaacgcggcgcgcaccttgggctcccacgcgcgcagcacttccaggtcgtcc

SEQ ID NO: 5 - DNA, 201 bp, Cupriavidus necator
  source 1..201: mol_type = unassigned DNA
gcagcttgatgccattgacgaggtagatggaaaccggcacgtgctctttgcgcagcgcgttcaggaacgggccttgtagcagttgccctttgttgctcatggcacactccaaatttataggtttagtggtgaatgatggggatggaaatccccggttcaagtcaggcggcgcaaaaacgcgccagaaaaaagatcaaaaac

SEQ ID NO: 6 - DNA, 479 bp, Cupriavidus necator
  source 1..479: mol_type = unassigned DNA
gaggatgccatgtccgaagcgcctgtccttgccccctcgacctcaacccagccgcccgccgccggccagctcaacctgatccgcccgcagccatatgccgactgggcgccgcaggtcacggccgaagaacgcgccacgctgcgccgcgagctggagcagggcgccgtgctgtacttcccgaacctgaatttccgcttccagccgggcgaagagcgcttccttgacagccgctattccgacggcaagtccaagaacatcaacctgcgcgccgacgacaccgcggtgcgcggcgcccagggcagtccgcaggacctggcggacctgtacacgctgatccgccgctacgccgacaacagcgaattgctggtgcgcacgctgttccctgaatacatcccgcacatgacgcgcgccggcacctcgctgcggcccagcgagatcgccgggcgcccggtcagctggcgcaaggacgacacccgcct

(19) United States
(12) Patent Application Publication
(10) Pub. No.: US 2024/0368645 A1
(43) Pub. Date: Nov. 7, 2024

(54) CARBON EFFICIENT TWO-PHASE HIGH PRODUCTIVITY FERMENTATION SYSTEM

(71) Applicant: PERSIDENT AND FELLOWS OF HARVARD COLLEGE, Cambridge, MA (US)

(72) Inventors: Ciaran DUNN, Cambridge, MA (US); Shannon Noel MANGLE, Cambridge, MA (US); Marika ZIESACK, Cambridge, MA (US)

(73) Assignee: PERSIDENT AND FELLOWS OF HARVARD COLLEGE, Cambridge, MA (US)

(21) Appl. No.: 18/294,871

(22) PCT Filed: Aug. 4, 2022
(86) PCT No.: PCT/US2022/074519

§ 371 Date: Feb. 2, 2024

Related U.S. Application Data
(60) Provisional application No. 63/230,400, filed on Aug. 6, 2021.

(51) Int. Cl. C12P 7/6463; C12M 1/00

(52) U.S. Cl. CPC C12P 7/6463; C12M 23/40; C12M 23/58; C12M 29/08

(57) ABSTRACT

The technology described herein is directed to systems and methods for producing a bioproduct from microorganisms such as bacteria. The system can comprise a growth phase and a production phase, that can occur in the same or different bioreactor chambers; the growth phase can use using gas fermentation or mixotrophic fermentation, and the production phase can use gas fermentation, mixotrophic fermentation, or organic carbon fermentation. In one example, the system can comprise at least one primary reactor chamber using gas fermentation or mixotrophic fermentation and at least one secondary reactor chamber using gas fermentation, mixotrophic fermentation, or organic carbon fermentation. Such systems can use bacteria that are capable of both autotrophy and heterotrophy and capable of switching between autotrophy and heterotrophy.

### CROSS-REFERENCE TO RELATED APPLICATIONS

This application claims benefit under 35 U.S.C. § 119 (e) of U.S. Provisional Application No. 63/230,400 filed Aug. 6, 2021, the contents of which are incorporated herein by reference in their entirety.

### GOVERNMENT SUPPORT

This invention was made with government support under DE-AR0001509 awarded by U.S. Department of Energy (DOE). The government has certain rights in this invention.

### SEQUENCE LISTING

The instant application contains a Sequence Listing which has been submitted in XML format via EFS-Web and is hereby incorporated by reference in its entirety. Said XML copy, created on Aug. 3, 2022, is named 002806-190360WOPT_SL.xml and is 11,359 bytes in size.

### TECHNICAL FIELD

The technology described herein relates to bacterial fermentation systems and methods.

### BACKGROUND

A sustainable future relies on minimizing the use of petro chemicals and reducing greenhouse gas emissions. One way to accomplish this goal is through increasing the usage of sustainable bioproducts from microorganisms, i.e., microbial bioproduction. Traditional microbial bioproduction utilizes carbohydrate-based feedstocks, but some of the cheapest and most sustainable feedstocks are gases (e.g., CO, CO₂, H₂, CH₄) from various point sources (e.g., steel mills, ethanol production plants, steam reforming plants, biogas). Compared to commonly used carbohydrate-based feedstocks, gaseous feedstocks are more cost-effective, are less land-intensive, have fewer restrictions to delivery in large volumes, and have smaller carbon footprints.

C. necator H₁₆ (formerly known as Ralstonia eutropha H₁₆) is an attractive species for industrial gas fermentation. It is a facultative chemolithotrophic bacterium that derives its energy from H₂ and carbon from CO₂, is genetically tractable, can be cultured with inexpensive minimal media components, is non-pathogenic, has a high-flux carbon storage pathway, and fixes the majority of fed CO₂ into biomass. However, many previous C. necator bioproduction methods have relied upon carbohydrate-based feedstocks (see e.g., U.S. Pat. No. 7,622,277; EP U.S. Pat. No. 2,935,599; Green et al. Biomacromolecules. 2002 January-February 3 (1); 208-13; Brigham et al. Deletion of Glyoxylate Shunt Pathway Genes Results in a 3-Hydroxybutyrate Overproducing Strain of Ralstonia eutropha. 2015 Synthetic Biology: Engineering, Evolution & Design. Poster Abstract 17; p. 32; the content of each of which is incorporated by reference in its entirety).

Current bioproduction platforms have limitations with regard to carbon efficiency, product versatility and/or productivity. Corn ethanol has high productivity but is carbon inefficient. Acetogenic ethanol production has achieved commercial scale and is a great alternative for low carbon alcohols and acids. Algal biodiesel production was considered a path for higher carbon fuels but has yet to achieve commercial viability. There is thus a great need for bioproduction platforms that can balance carbon efficiency, product versatility, and productivity.

### SUMMARY

Described herein are bioreactor systems and methods for producing a bioproduct from a microorganism (e.g., bacterium). Such systems and methods use microorganisms (e.g., bacteria) that are capable of both organic carbon fermentation and gas fermentation, commonly referred to as mixotrophs. Importantly, such microorganisms (e.g., bacteria) are also capable of switching between organic carbon fermentation and gas fermentation, referred to herein as switchotrophs. The bioreactors described herein can comprise at least one reactor chamber that induces gas fermentation and at least one reactor chamber that induces carbon fermentation. An exemplary system is a hybrid of continuous gas fermentation (H₂/O₂/CO₂) for biomass production and subsequent fed-batch mixotrophic fermentation (sugar and H₂).

The systems and methods described herein exhibit at least the following benefits compared to other bioproduction platforms: (1) gas feedstocks are more cost-effective, less land-intensive, have fewer restrictions to delivery in large volumes, and have smaller carbon footprints compared to carbohydrate-based feedstocks: (2) gas fermentation provides an austere environment unfavorable to contamination by other microorganisms: (3) the gas fermentation minimizes genetic drift since it is used solely to produce biomass: (4) the mixotrophic fermentation can use hydrogen to draw down any released CO₂ from growth on sugar, thus optimizing production of the bioproduct and minimizing CO₂ output: (5) the mixotrophic fermentation can minimize genetic drift since it is optimized for bioproduct product, not microbial (e.g., bacterial) growth: (6) the system is capable of producing a wide range of bioproducts; and/or (7) this approach addresses the limitations that other technologies face in feedstocks, productivity, and product tailoring, thus unlocking increased scale, improved economics, and meaningful sustainability.

Accordingly, in one aspect described herein is a system for producing a bioproduct comprising: at least one reactor chamber containing therein at least one solution selected from: (a) at least one growth solution comprising: (i) carbon dioxide (CO₂), hydrogen (H₂), and oxygen (O₂); or (ii) an organic carbon source, hydrogen (H₂), and oxygen (O₂), and optionally carbon dioxide (CO₂); and/or (b) at least one production solution comprising: (i) carbon dioxide (CO₂), hydrogen (H₂), and oxygen (O₂); (ii) an organic carbon source, hydrogen (H₂), and oxygen (O₂), and optionally carbon dioxide (CO₂); or (iii) an organic carbon source and oxygen (O₂); and wherein the at least one reactor chamber contains therein: (c) at least one microorganism (e.g., bacterium) in the at least one growth solution and/or at least one production solution, wherein the at least one microorganism (e.g., bacterium) produces the bioproduct.

In some embodiments of any of the aspects, the system comprises one reactor chamber.

In some embodiments of any of the aspects, at least a portion of the growth solution can be removed from the at least one reactor chamber.

In some embodiments of any of the aspects, at least a portion of the production solution can be added to the at least one reactor chamber.

In some embodiments of any of the aspects, the system comprises at least two reactor chambers.

In some embodiments of any of the aspects, the at least one reactor chamber containing the at least one growth solution is a continuous fermentation reactor chamber.

In some embodiments of any of the aspects, the at least one reactor chamber containing the at least one growth solution is a gas fermentation reactor chamber.

In some embodiments of any of the aspects, the at least one reactor chamber containing the at least one growth solution is a mixotrophic fermentation reactor chamber.

In some embodiments of any of the aspects, the at least one reactor chamber containing the at least one production solution is a fed-batch fermentation reactor chamber.

In some embodiments of any of the aspects, the at least one reactor chamber containing the least one production solution is: (a) a gas fermentation reactor chamber; (b) a gas and organic carbon (mixotrophic) fermentation reactor chamber; or (c) an organic carbon fermentation reactor chamber.

In some embodiments of any of the aspects, the at least one reactor chamber containing the at least one production solution emits no CO₂.

In some embodiments of any of the aspects, the at least one reactor chamber containing the at least one production solution emits no CO₂.

In some embodiments of any of the aspects, the at least one reactor chamber containing the at least one production solution emits at most 1 molecule of CO₂ per molecule of acetyl-CoA.

In some embodiments of any of the aspects, the at least one reactor chamber further comprises a pair of electrodes in contact with the first and/or at least one production solution that split water to form the hydrogen.

In some embodiments of any of the aspects, the at least one reactor chamber further comprises an isolated gas volume above a surface of the first and/or at least one production solution within a headspace of the at least one reactor chamber.

In some embodiments of any of the aspects, the isolated gas volume comprises carbon dioxide (CO₂), hydrogen (H₂), and/or oxygen (O₂).

In some embodiments of any of the aspects, the at least one reactor chamber further comprises a power source comprising a renewable source of energy.

In some embodiments of any of the aspects, the renewable source of energy comprises a solar cell, wind turbine, generator, battery, or grid power.

In one aspect described herein is a system for producing a bioproduct comprising: (a) a primary reactor chamber with a at least one growth solution contained therein, wherein the at least one growth solution comprises: (i) carbon dioxide (CO₂), hydrogen (H₂), and oxygen (O₂), or (ii) an organic carbon source, hydrogen (H₂), and oxygen (O₂), and optionally carbon dioxide (CO₂); (b) at least one secondary reactor chamber with a at least one production solution contained therein, wherein the at least one production solution comprises: (i) carbon dioxide (CO₂), hydrogen (H₂), and oxygen (O₂); (ii) an organic carbon source, hydrogen (H₂), and oxygen (O₂), and optionally carbon dioxide (CO₂); or (iii) an organic carbon source and oxygen (O₂); and (c) at least one microorganism (e.g., bacterium) in the at least one growth solution of the primary reactor chamber and/or at least one production solution of the secondary reactor chamber, wherein the at least one microorganism (e.g., bacterium) produces the bioproduct.

In some embodiments of any of the aspects, the system comprises at least two secondary reactor chambers.

In some embodiments of any of the aspects, the system comprises three secondary reactor chambers, wherein: (a) the solution in the first secondary reactor chamber comprises carbon dioxide (CO₂), hydrogen (H₂), and oxygen (O₂); (b) the solution in the second secondary reactor chamber comprises an organic carbon source, hydrogen (H₂), and oxygen (O₂); and (c) the solution in the third secondary reactor chamber comprises an organic carbon source and oxygen (O₂).

In some embodiments of any of the aspects, the primary reactor chamber is a continuous fermentation reactor chamber.

In some embodiments of any of the aspects, the primary reactor chamber is a gas fermentation reactor chamber.

In some embodiments of any of the aspects, the primary reactor chamber is a mixotrophic fermentation reactor chamber.

In some embodiments of any of the aspects, the secondary reactor chamber is a fed-batch fermentation reactor chamber.

In some embodiments of any of the aspects, the primary and at least one secondary reactor chambers are physically linked.

In some embodiments of any of the aspects, the at least one growth solution from the primary reactor chamber is batch fed into the secondary reactor chamber.

In some embodiments of any of the aspects, the secondary reactor chamber is: (a) a gas fermentation reactor chamber; (b) a gas and organic carbon (mixotrophic) fermentation reactor chamber; or (c) an organic carbon fermentation reactor chamber.

In some embodiments of any of the aspects, the primary reactor chamber emits no CO₂.

In some embodiments of any of the aspects, the secondary reactor chamber emits no CO₂.

In some embodiments of any of the aspects, the secondary reactor chamber emits at most 1 molecule of CO₂ per molecule of acetyl-CoA.

In some embodiments of any of the aspects, the primary and/or secondary reactor chamber further comprises a pair of electrodes in contact with the first and/or at least one production solution that split water to form the hydrogen.

In some embodiments of any of the aspects, the primary and/or secondary reactor chamber further comprises an isolated gas volume above a surface of the at least one growth solution and/or at least one production solution within a headspace of the primary and/or secondary reactor chamber.

In some embodiments of any of the aspects, the isolated gas volume comprises carbon dioxide (CO₂), hydrogen (H₂), and/or oxygen (O₂).

In some embodiments of any of the aspects, the primary and/or secondary reactor chamber further comprises a power source comprising a renewable source of energy.

In some embodiments of any of the aspects, the renewable source of energy comprises a solar cell, wind turbine, generator, battery, or grid power.

In some embodiments of any of the aspects, the system comprises: (a) one growth solution and one production solution: (b) two growth solutions and one production solution; or (c) one growth solution and two production solutions.

In some embodiments of any of the aspects, the system further comprises at least one inducer solution.

In some embodiments of any of the aspects, the at least one inducer solution comprises a level of bioavailable nitrogen below a pre-determined threshold.

In some embodiments of any of the aspects, the at least one inducer solution comprises arabinose.

In some embodiments of any of the aspects, the at least one inducer solution further comprises: (a) carbon dioxide (CO₂), hydrogen (H₂), and oxygen (O₂); (b) an organic carbon source, hydrogen (H₂), and oxygen (O₂), and optionally carbon dioxide (CO₂); or (c) an organic carbon source and oxygen (O₂).

In some embodiments of any of the aspects, the microorganism (e.g., bacterium) is a chemolithotroph.

In some embodiments of any of the aspects, the microorganism (e.g., bacterium) is a mixotroph.

In some embodiments of any of the aspects, the mixotroph is capable of gas fermentation and organic carbon fermentation.

In some embodiments of any of the aspects, the microorganism (e.g., bacterium) is a switchotroph.

In some embodiments of any of the aspects, the switchotroph is capable of switching between gas fermentation and organic carbon fermentation.

In some embodiments of any of the aspects, the microorganism (e.g., bacterium) is not a heterotroph.

In some embodiments of any of the aspects, the microorganism (e.g., bacterium) is Cupriavidus necator.

In some embodiments of any of the aspects, the microorganism (e.g., bacterium) naturally produces the bioproduct.

In some embodiments of any of the aspects, the microorganism (e.g., bacterium) is engineered to produce the bioproduct.

In some embodiments of any of the aspects, the microorganism (e.g., bacterium) is capable of being induced to produce the bioproduct.

In some embodiments of any of the aspects, the bioproduct is capable of being isolated, collected, or concentrated after the microorganism (e.g., bacterium) produces a pre-determined concentration of the bioproduct.

In some embodiments of any of the aspects, the organic carbon source is selected from the group consisting of: glucose, glycerol, gluconate, acetate, fructose, decanoate, fatty acid, and glycerol gluconate.

In some embodiments of any of the aspects, the organic carbon source comprises glucose.

In some embodiments of any of the aspects, the at least one growth solution and/or at least one production solution comprises cell culture medium.

In some embodiments of any of the aspects, the at least one growth solution and/or at least one production solution comprises defined medium.

In some embodiments of any of the aspects, the at least one growth solution and/or at least one production solution comprises minimal medium.

In some embodiments of any of the aspects, the at least one growth solution and/or at least one production solution comprises rich medium.

In some embodiments of any of the aspects, the bioproduct is selected from the group consisting of: polypeptide, glycoprotein, lipoprotein, lipid, monosaccharide, polysaccharide, nucleic acid, small molecule, or metabolite.

In some embodiments of any of the aspects, the bioproduct is selected from the group consisting of: polyhydroxyalkanoate (PHA); sucrose: lipochitooligosaccharide; and triacylglyceride.

In one aspect described herein is a method of a culturing a microorganism (e.g., bacterium), the method comprising: (a) culturing the microorganism (e.g., bacterium) in at least one reactor chamber with at least one growth solution contained therein, wherein the at least one growth solution comprises: (i) carbon dioxide (CO₂), hydrogen (H₂), and oxygen (O₂); or (ii) an organic carbon source, hydrogen (H₂), and oxygen (O₂), and optionally carbon dioxide (CO₂); (b) adding at least one production solution to the at least one reactor chamber, wherein the at least one production solution comprises: (i) carbon dioxide (CO₂), hydrogen (H₂), and oxygen (O₂); (ii) an organic carbon source, hydrogen (H₂), and oxygen (O₂), and optionally carbon dioxide (CO₂); or (iii) an organic carbon source and oxygen (O₂); and (c) culturing the microorganism (e.g., bacterium) in the at least one production solution.

In one aspect described herein is a method of a culturing a microorganism (e.g., bacterium), comprising: (a) culturing the microorganism (e.g., bacterium) in at least one reactor chamber with at least one growth solution contained therein, wherein the at least one growth solution comprises: (i) carbon dioxide (CO₂), hydrogen (H₂), and oxygen (O₂); or (ii) an organic carbon source, hydrogen (H₂), and oxygen (O₂), and optionally carbon dioxide (CO₂); (b) adding at least one production solution to the at least one reactor chamber, wherein the at least one production solution comprises: (i) carbon dioxide (CO₂), hydrogen (H₂), and oxygen (O₂); (ii) an organic carbon source, hydrogen (H₂), and oxygen (O₂), and optionally carbon dioxide (CO₂); or (iii) an organic carbon source and oxygen (O₂); (c) culturing the microorganism (e.g., bacterium) in the at least one production solution; and (d) isolating, collecting, or concentrating the bioproduct from the microorganism (e.g., bacterium) in the at least one reactor chamber or from the at least one production solution in the at least one reactor chamber.

In some embodiments of any of the aspects, the microorganism (e.g., bacterium) is cultured in the at least one growth solution for a sufficient amount of time and under sufficient conditions for the microorganism (e.g., bacterium) to grow to a pre-determined concentration.

In some embodiments of any of the aspects, the microorganism (e.g., bacterium) does not produce the bioproduct in the at least one growth solution.

In some embodiments of any of the aspects, at least a portion of the at least one growth solution is removed from the at least one reactor chamber after the microorganism (e.g., bacterium) grows to a pre-determined concentration.

In some embodiments of any of the aspects, at least a portion of the at least one production solution is added to the at least one reactor chamber after the microorganism (e.g., bacterium) grows to a pre-determined concentration.

In some embodiments of any of the aspects, at least a portion of the at least one growth solution is removed from the at least one reactor chamber whenever the microorganism (e.g., bacterium) grows to a pre-determined concentration such that the microorganism (e.g., bacterium) does not ever exceed the pre-determined concentration.

In some embodiments of any of the aspects, at least a portion of the at least one production solution is added to the at least one reactor chamber whenever the microorganism (e.g., bacterium) grows to a pre-determined concentration such that the microorganism (e.g., bacterium) does not ever exceed the pre-determined concentration.

In some embodiments of any of the aspects, the microorganism (e.g., bacterium) is cultured in the at least one production solution for a sufficient amount of time and under sufficient conditions for the microorganism (e.g., bacterium) to produce a pre-determined concentration of the bioproduct.

In some embodiments of any of the aspects, the microorganism (e.g., bacterium) does not exhibit substantial growth in the at least one production solution.

In some embodiments of any of the aspects, the at least one reactor chamber containing the at least one growth solution is a continuous fermentation reactor chamber.

In some embodiments of any of the aspects, the at least one reactor chamber containing the at least one growth solution is a gas fermentation reactor chamber.

In some embodiments of any of the aspects, the at least one reactor chamber containing the at least one growth solution is a mixotrophic fermentation reactor chamber.

In some embodiments of any of the aspects, the at least one reactor chamber containing the at least one production solution is a fed-batch fermentation reactor chamber.

In some embodiments of any of the aspects, the at least one reactor chamber containing the at least one production solution is: (a) a gas fermentation reactor chamber; (b) a gas and organic carbon (mixotrophic) fermentation reactor chamber; or (c) an organic carbon fermentation reactor chamber.

In some embodiments of any of the aspects, the at least one reactor chamber containing the at least one growth solution emits no CO₂.

In some embodiments of any of the aspects, the at least one reactor chamber containing the at least one production solution emits no CO₂.

In some embodiments of any of the aspects, the at least one reactor chamber containing the at least one production solution emits at most 1 molecule of CO₂ per molecule of acetyl-CoA.

In some embodiments of any of the aspects, the at least one reactor chamber further comprises a pair of electrodes in contact with the first and/or at least one production solution that split water to form the hydrogen.

In some embodiments of any of the aspects, the at least one reactor chamber further comprises an isolated gas volume above a surface of the first and/or at least one production solution within a headspace of the at least one reactor chamber.

In some embodiments of any of the aspects, the isolated gas volume comprises carbon dioxide (CO₂), hydrogen (H₂), and/or oxygen (O₂).

In some embodiments of any of the aspects, the at least one reactor chamber further comprises a power source comprising a renewable source of energy.

In some embodiments of any of the aspects, the renewable source of energy comprises a solar cell, wind turbine, generator, battery, or grid power.

In one aspect described herein is a method of a culturing a microorganism (e.g., bacterium), the method comprising: (a) culturing the microorganism (e.g., bacterium) in a primary reactor chamber with a at least one growth solution contained therein, wherein the at least one growth solution comprises: (i) carbon dioxide (CO₂), hydrogen (H₂), and oxygen (O₂); or (ii) an organic carbon source, hydrogen (H₂), and oxygen (O₂), and optionally carbon dioxide (CO₂); (b) moving at least a portion of the at least one growth solution from the primary reactor chamber into at least one secondary reactor chamber with a at least one production solution contained therein, wherein the at least one production solution comprises: (i) carbon dioxide (CO₂), hydrogen (H₂), and oxygen (O₂); (ii) an organic carbon source, hydrogen (H₂), and oxygen (O₂), and optionally carbon dioxide (CO₂); or (iii) an organic carbon source and oxygen (O₂); and (c) culturing the microorganism (e.g., bacterium) in the secondary reactor chamber.

In one aspect described herein is a method of producing a bioproduct, comprising: (a) culturing a microorganism (e.g., bacterium) that produces a bioproduct in a primary reactor chamber with a at least one growth solution contained therein, wherein the at least one growth solution comprises: (i) carbon dioxide (CO₂), hydrogen (H₂), and oxygen (O₂); or (ii) an organic carbon source, hydrogen (H₂), and oxygen (O₂), and optionally carbon dioxide (CO₂); (b) moving at least a portion of the at least one growth solution from the primary reactor chamber into a secondary reactor chamber with a at least one production solution contained therein, wherein the at least one production solution comprises: (i) carbon dioxide (CO₂), hydrogen (H₂), and oxygen (O₂); (ii) an organic carbon source, hydrogen (H₂), and oxygen (O₂), and optionally carbon dioxide (CO₂); or (iii) an organic carbon source and oxygen (O₂); (c) culturing the microorganism (e.g., bacterium) in the secondary reactor chamber; and (d) isolating, collecting, or concentrating the bioproduct from the microorganism (e.g., bacterium) in the secondary reactor chamber or from the at least one production solution in the second reactor chamber.

In some embodiments of any of the aspects, the microorganism (e.g., bacterium) is cultured in the primary reactor chamber for a sufficient amount of time and under sufficient conditions for the microorganism (e.g., bacterium) to grow to a pre-determined concentration.

In some embodiments of any of the aspects, the microorganism (e.g., bacterium) does not produce the bioproduct in the primary reactor chamber.

In some embodiments of any of the aspects, at least a portion of the at least one growth solution from the primary reactor chamber is moved into the at least one secondary reactor chamber after the microorganism (e.g., bacterium) grows to a pre-determined concentration.

In some embodiments of any of the aspects, the method comprises the following iterative steps: (a) moving at least a portion of the at least one growth solution from the primary reactor chamber into a first secondary reactor chamber after the microorganism (e.g., bacterium) grows to a pre-determined concentration; and (b) moving at least a portion of the at least one growth solution from the primary reactor chamber into a second secondary reactor chamber after the microorganism (e.g., bacterium) grows to a pre-determined concentration.

In some embodiments of any of the aspects, the method comprises the following iterative steps: (a) moving at least a portion of the at least one growth solution from the primary reactor chamber into a first secondary reactor chamber after the microorganism (e.g., bacterium) grows to a pre-determined concentration: (b) moving at least a portion of the at least one growth solution from the primary reactor chamber into a second secondary reactor chamber after the microorganism (e.g., bacterium) grows to a pre-determined concentration; and (c) moving at least a portion of the at least one growth solution from the primary reactor chamber into a third secondary reactor chamber after the microorganism (e.g., bacterium) grows to a pre-determined concentration.

In some embodiments of any of the aspects, a portion of the at least one growth solution from the primary reactor chamber is moved into at least one secondary reactor chamber whenever the microorganism (e.g., bacterium) grows to a pre-determined concentration such that the microorganism (e.g., bacterium) does not ever exceed the pre-determined concentration.

In some embodiments of any of the aspects, the microorganism (e.g., bacterium) is cultured in the secondary reactor chamber for a sufficient amount of time and under sufficient conditions for the microorganism (e.g., bacterium) to produce a pre-determined concentration of the bioproduct.

In some embodiments of any of the aspects, the microorganism (e.g., bacterium) does not exhibit substantial growth in the at least one secondary reactor chamber.

In some embodiments of any of the aspects, the primary reactor chamber is a continuous fermentation reactor chamber.

In some embodiments of any of the aspects, the primary reactor chamber is a gas fermentation reactor chamber.

In some embodiments of any of the aspects, the primary reactor chamber is a mixotrophic fermentation reactor chamber.

In some embodiments of any of the aspects, the secondary reactor chamber is a fed-batch fermentation reactor chamber.

In some embodiments of any of the aspects, the primary and at least one secondary reactor chambers are physically linked.

In some embodiments of any of the aspects, the at least one growth solution from the primary reactor chamber is batch fed into the secondary reactor chamber.

In some embodiments of any of the aspects, the secondary reactor chamber is: (a) a gas fermentation reactor chamber; (b) a gas and organic carbon (mixotrophic) fermentation reactor chamber; or (c) an organic carbon fermentation reactor chamber.

In some embodiments of any of the aspects, the primary reactor chamber emits no CO₂.

In some embodiments of any of the aspects, the secondary reactor chamber emits no CO₂.

In some embodiments of any of the aspects, the secondary reactor chamber emits at most 1 molecule of CO₂ per molecule of acetyl-CoA.

In some embodiments of any of the aspects, the primary and/or secondary reactor chamber further comprises a pair of electrodes in contact with the at least one growth solution and/or at least one production solution that split water to form the hydrogen.

In some embodiments of any of the aspects, the primary and/or secondary reactor chamber further comprises an isolated gas volume above a surface of the at least one growth solution and/or at least one production solution within a headspace of the primary and/or secondary reactor chamber.

In some embodiments of any of the aspects, the isolated gas volume comprises carbon dioxide (CO₂), hydrogen (H₂), and/or oxygen (O₂).

In some embodiments of any of the aspects, the primary and/or secondary reactor chamber further comprises a power source comprising a renewable source of energy.

In some embodiments of any of the aspects, the renewable source of energy comprises a solar cell, wind turbine, generator, battery, or grid power.

In some embodiments of any of the aspects, the method comprises using: (a) one growth solution and one production solution: (b) two growth solutions and one production solution; or (c) one growth solution and two production solutions.

In some embodiments of any of the aspects, the method further comprises adding at least one inducer solution to the at least one reactor chamber;

In some embodiments of any of the aspects, the at least one inducer solution is added after the microorganism (e.g., bacterium) is cultured in the at least one growth solution for a sufficient amount of time and under sufficient conditions for the microorganism (e.g., bacterium) to grow to a pre-determined concentration.

In some embodiments of any of the aspects, the inducer solution induces the microorganism (e.g., bacterium) to produce the bioproduct.

In some embodiments of any of the aspects, the at least one inducer solution comprises a level of bioavailable nitrogen below a pre-determined threshold.

In some embodiments of any of the aspects, the at least one inducer solution comprises arabinose.

In some embodiments of any of the aspects, the at least one inducer solution further comprises: (a) carbon dioxide (CO₂), hydrogen (H₂), and oxygen (O₂); (b) an organic carbon source, hydrogen (H₂), and oxygen (O₂), and optionally carbon dioxide (CO₂); or (c) an organic carbon source and oxygen (O₂).

In some embodiments of any of the aspects, the microorganism (e.g., bacterium) is a chemolithotroph.

In some embodiments of any of the aspects, the microorganism (e.g., bacterium) is a mixotroph.

In some embodiments of any of the aspects, the mixotroph is capable of gas fermentation and organic carbon fermentation.

In some embodiments of any of the aspects, the microorganism (e.g., bacterium) is a switchotroph.

In some embodiments of any of the aspects, the switchotroph is capable of switching between gas fermentation and organic carbon fermentation.

In some embodiments of any of the aspects, the microorganism (e.g., bacterium) is not a heterotroph.

In some embodiments of any of the aspects, the microorganism (e.g., bacterium) is Cupriavidus necator.

In some embodiments of any of the aspects, the microorganism (e.g., bacterium) naturally produces the bioproduct.

In some embodiments of any of the aspects, the microorganism (e.g., bacterium) is engineered to produce the bioproduct.

In some embodiments of any of the aspects, the bioproduct is isolated, collected, or concentrated after the microorganism (e.g., bacterium) produces a pre-determined concentration of the bioproduct.

In some embodiments of any of the aspects, the organic carbon source is selected from the group consisting of: glucose, glycerol, gluconate, acetate, fructose, decanoate, fatty acid, and glycerol gluconate.

In some embodiments of any of the aspects, the organic carbon source comprises glucose.

In some embodiments of any of the aspects, the at least one growth solution and/or at least one production solution comprises cell culture medium.

In some embodiments of any of the aspects, the at least one growth solution and/or at least one production solution comprises defined medium.

In some embodiments of any of the aspects, the at least one growth solution and/or at least one production solution comprises minimal medium.

In some embodiments of any of the aspects, the at least one growth solution and/or at least one production solution comprises rich medium.

In some embodiments of any of the aspects, the bioproduct is selected from the group consisting of: polypeptide, glycoprotein, lipoprotein, lipid, monosaccharide, polysaccharide, nucleic acid, small molecule, or metabolite.

In some embodiments of any of the aspects, the bioproduct is selected from the group consisting of: polyhydroxyalkanoate (PHA); sucrose: lipochitooligosaccharide; and triacylglyceride.

In one aspect described herein is a method of adapting the metabolism of a microorganism (e.g., bacterium) for gas fermentation, the method comprising: (a) culturing the microorganism (e.g., bacterium) in a solution comprising an organic carbon source; and (b) transitioning the microorganism (e.g., bacterium) to a gas fermentation solution lacking an organic carbon source once the microorganism (e.g., bacterium) grows to a pre-determined concentration.

In some embodiments of any of the aspects, the organic carbon source is selected from the group consisting of: glucose, glycerol, gluconate, acetate, fructose, decanoate, fatty acid, and glycerol gluconate.

In one aspect described herein is a system for producing a bioproduct comprising: (a) a primary reactor chamber with a first solution contained therein, wherein the first solution comprises carbon dioxide (CO₂), hydrogen (H₂), and oxygen (O₂); (b) at least one secondary reactor chamber with a second solution contained therein, wherein the second solution comprises: (i) carbon dioxide (CO₂), hydrogen (H₂), and oxygen (O₂); (ii) an organic carbon source, hydrogen (H₂), and oxygen (O₂); or (iii) an organic carbon source and oxygen (O₂); and (c) at least one microorganism (e.g., bacterium) in the first solution of the primary reactor chamber and/or second solution of the secondary reactor chamber, wherein the at least one microorganism (e.g., bacterium) produces the bioproduct.

In some embodiments of any of the aspects, the system comprises at least two secondary reactor chambers.

In some embodiments of any of the aspects, the system comprises three secondary reactor chambers, wherein: (a) the solution in the first secondary reactor chamber comprises carbon dioxide (CO₂), hydrogen (H₂), and oxygen (O₂); (b) the solution in the second secondary reactor chamber comprises an organic carbon source, hydrogen (H₂), and oxygen (O₂); and (c) the solution in the third secondary reactor chamber comprises an organic carbon source and oxygen (O₂).

In one aspect described herein is a method of a culturing a microorganism (e.g., bacterium), comprising: (a) culturing the microorganism (e.g., bacterium) in a primary reactor chamber with a first solution contained therein, wherein the first solution comprises carbon dioxide (CO₂), hydrogen (H₂), and oxygen (O₂); (b) moving at least a portion of the first solution from the primary reactor chamber into at least one secondary reactor chamber with a second solution contained therein, wherein the second solution comprises: (i) carbon dioxide (CO₂), hydrogen (H₂), and oxygen (O₂); (ii) an organic carbon source, hydrogen (H₂), and oxygen (O₂); or (iii) an organic carbon source and oxygen (O₂); and (c) culturing the microorganism (e.g., bacterium) in the secondary reactor chamber.

In one aspect described herein is a method of producing a bioproduct, comprising: (a) culturing a microorganism (e.g., bacterium) that produces a bioproduct in a primary reactor chamber with a first solution contained therein, wherein the first solution comprises carbon dioxide (CO₂), hydrogen (H₂), and oxygen (O₂); (b) moving at least a portion of the first solution from the primary reactor chamber into a secondary reactor chamber with a second solution contained therein, wherein the second solution comprises: (i) carbon dioxide (CO₂), hydrogen (H₂), and oxygen (O₂); (ii) an organic carbon source, hydrogen (H₂), and oxygen (O₂); or (iii) an organic carbon source and oxygen (O₂); (c) culturing the microorganism (e.g., bacterium) in the secondary reactor chamber; and (d) isolating, collecting, or concentrating the bioproduct from the microorganism (e.g., bacterium) in the secondary reactor chamber or from the second solution in the second reactor chamber.

In some embodiments of any of the aspects, the microorganism (e.g., bacterium) is a chemolithotroph.

In some embodiments of any of the aspects, the microorganism (e.g., bacterium) is a mixotroph.

In some embodiments of any of the aspects, the mixotroph is capable of gas fermentation and organic carbon fermentation.

In some embodiments of any of the aspects, the microorganism (e.g., bacterium) is a switchotroph.

In some embodiments of any of the aspects, the switchotroph is capable of switching between gas fermentation and organic carbon fermentation.

In some embodiments of any of the aspects, the microorganism (e.g., bacterium) is not a heterotroph.

In some embodiments of any of the aspects, the microorganism (e.g., bacterium) is Cupriavidus necator.

In some embodiments of any of the aspects, the microorganism (e.g., bacterium naturally produces the bioproduct.

In some embodiments of any of the aspects, the microorganism (e.g., bacterium) is engineered to produce the bioproduct.

In some embodiments of any of the aspects, the microorganism (e.g., bacterium) is cultured in the primary reactor chamber for a sufficient amount of time and under sufficient conditions for the microorganism (e.g., bacterium) to grow to a pre-determined concentration.

In some embodiments of any of the aspects, the microorganism (e.g., bacterium) does not produce the bioproduct in the primary reactor chamber.

In some embodiments of any of the aspects, at least a portion of the first solution from the primary reactor chamber is moved into the at least one secondary reactor chamber after the microorganism (e.g., bacterium) grows to a pre-determined concentration.

In some embodiments of any of the aspects, the method comprises the following iterative steps: (a) a portion of the first solution from the primary reactor chamber is moved into a first secondary reactor chamber after the microorganism (e.g., bacterium) grows to a pre-determined concentration; and (b) a portion of the first solution from the primary reactor chamber is moved into a second secondary reactor chamber after the microorganism (e.g., bacterium) grows to a pre-determined concentration.

In some embodiments of any of the aspects, the method comprises the following iterative steps: (a) a portion of the first solution from the primary reactor chamber is moved into a first secondary reactor chamber after the microorganism (e.g., bacterium) grows to a pre-determined concentration: (b) a portion of the first solution from the primary reactor chamber is moved into a second secondary reactor chamber after the microorganism (e.g., bacterium) grows to a pre-determined concentration; and (c) a portion of the first solution from the primary reactor chamber is moved into a third secondary reactor chamber after the microorganism (e.g., bacterium) grows to a pre-determined concentration.

In some embodiments of any of the aspects, a portion of the first solution from the primary reactor chamber is moved into at least one secondary reactor chamber whenever the microorganism (e.g., bacterium) grows to a pre-determined concentration such that the microorganism (e.g., bacterium) does not ever exceed the pre-determined concentration.

In some embodiments of any of the aspects, the microorganism (e.g., bacterium) is cultured in the secondary reactor chamber for a sufficient amount of time and under sufficient conditions for the microorganism (e.g., bacterium) to produce a pre-determined concentration of the bioproduct.

In some embodiments of any of the aspects, the method further comprises inducing the microorganism (e.g., bacterium) to produce the bioproduct.

In some embodiments of any of the aspects, the microorganism (e.g., bacterium) does not exhibit substantial growth in the at least one secondary reactor chamber.

In some embodiments of any of the aspects, the bioproduct is isolated, collected, or concentrated after the microorganism (e.g., bacterium) produces a pre-determined concentration of the bioproduct.

In some embodiments of any of the aspects, the primary reactor chamber is a continuous fermentation reactor chamber.

In some embodiments of any of the aspects, the primary reactor chamber is a gas fermentation reactor chamber.

In some embodiments of any of the aspects, the secondary reactor chamber is a fed-batch fermentation reactor chamber.

In some embodiments of any of the aspects, the primary and at least one secondary reactor chambers are physically linked.

In some embodiments of any of the aspects, the first solution from the primary reactor chamber is batch fed into the secondary reactor chamber.

In some embodiments of any of the aspects, the secondary reactor chamber is: (a) a gas fermentation reactor chamber; (b) a gas and organic carbon (mixotrophic) fermentation reactor chamber; or (c) an organic carbon fermentation reactor chamber.

In some embodiments of any of the aspects, the second solution comprises: (a) at least 11 molecules of H₂ per molecule of acetyl-CoA: (b) at least ⅓ molecule of organic carbon source and 5/3 molecules of H₂ per molecule of acetyl-CoA; or (c) at least ½ molecule of organic carbon source per molecule of acetyl-CoA.

In some embodiments of any of the aspects, the second solution comprises: (a) at least 11 molecules of H₂ per molecule of acetyl-CoA: (b) at least 1 molecule of organic carbon source and 5 molecules of H₂ per 3 molecules of acetyl-CoA; or (c) at least 1 molecule of organic carbon source per 2 molecules of acetyl-CoA.

In some embodiments of any of the aspects, the organic carbon source comprises glucose, glycerol, gluconate, acetate, fructose, or decanoate.

In some embodiments of any of the aspects, the organic carbon source comprises glucose.

In some embodiments of any of the aspects, the primary reactor chamber emits no CO₂.

In some embodiments of any of the aspects, the secondary reactor chamber emits no CO₂.

In some embodiments of any of the aspects, the secondary reactor chamber emits at most 1 molecule of CO₂ per molecule of acetyl-CoA.

In some embodiments of any of the aspects, the first and/or second solution comprises cell culture medium.

In some embodiments of any of the aspects, the first and/or second solution comprises defined medium.

In some embodiments of any of the aspects, the first and/or second solution comprises minimal medium.

In some embodiments of any of the aspects, the first and/or second solution comprises rich medium.

In some embodiments of any of the aspects, the bioproduct is selected from the group consisting of: polypeptide, glycoprotein, lipoprotein, lipid, monosaccharide, polysaccharide, nucleic acid, small molecule, or metabolite.

In some embodiments of any of the aspects, the bioproduct is selected from the group consisting of: polyhydroxyalkanoate (PHA); sucrose: lipochitooligosaccharide; and triacylglyceride.

In some embodiments of any of the aspects, the primary and/or secondary reactor chamber further comprises a pair of electrodes in contact with the first and/or second solution that split water to form the hydrogen.

In some embodiments of any of the aspects, the primary and/or secondary reactor chamber further comprises an isolated gas volume above a surface of the first and/or second solution within a headspace of the primary and/or secondary reactor chamber.

In some embodiments of any of the aspects, the isolated gas volume comprises carbon dioxide (CO₂), hydrogen (H₂), and/or oxygen (O₂).

In some embodiments of any of the aspects, the primary and/or secondary reactor chamber further comprises a power source comprising a renewable source of energy.

In some embodiments of any of the aspects, the renewable source of energy comprises a solar cell, wind turbine, generator, battery, or grid power.

### BRIEF DESCRIPTION OF THE DRAWINGS

FIG. 1 is an exemplary schematic showing an overall metabolic engineering and fermentation strategy. In some embodiments, the first and second stages can occur sequentially in the same reaction chamber. In some embodiments, the first and second stages can occur in separate reaction chambers (e.g., primary and secondary reactor chambers). In some embodiments, the first stage is the growth fermenter that can be under continuous gas fermentation: in some embodiments the first stage can be under mixotrophic (gas and organic carbon, and optionally CO₂) fermentation. The second stage is a fed-batch strategy for production that can manifest as gas only, mixotrophic (gas and organic carbon, and optionally CO₂), or sugar (organic carbon) only fermentation. The relative requirements of energy in the form of H₂ or glucose indicate the degree of carbon efficiency of the system. “Gases only” is the most carbon efficient with no CO₂ release. “DSP” indicates downstream processing.

FIG. 2A-2C is a series of schematics showing the different modes of a two-stage bioprocess. Stage 1 can use a continuous gas fermentation process or a mixotrophy fermentation process to grow cells. Stage 2 is a discontinuous process that is used for bioproduct (e.g., triacylglyceride (TAG)) production either from gases (FIG. 2A), via mixotrophy (FIG. 2B) or from sugar (FIG. 2C). Addition of external hydrogen as a reducing equivalent allows for increased carbon efficiency. In some embodiments, stages 1 and 2 can occur sequentially in the same reaction chamber. In some embodiments, stages 1 and 2 can occur in separate reaction chambers (e.g., primary and secondary reactor chambers).

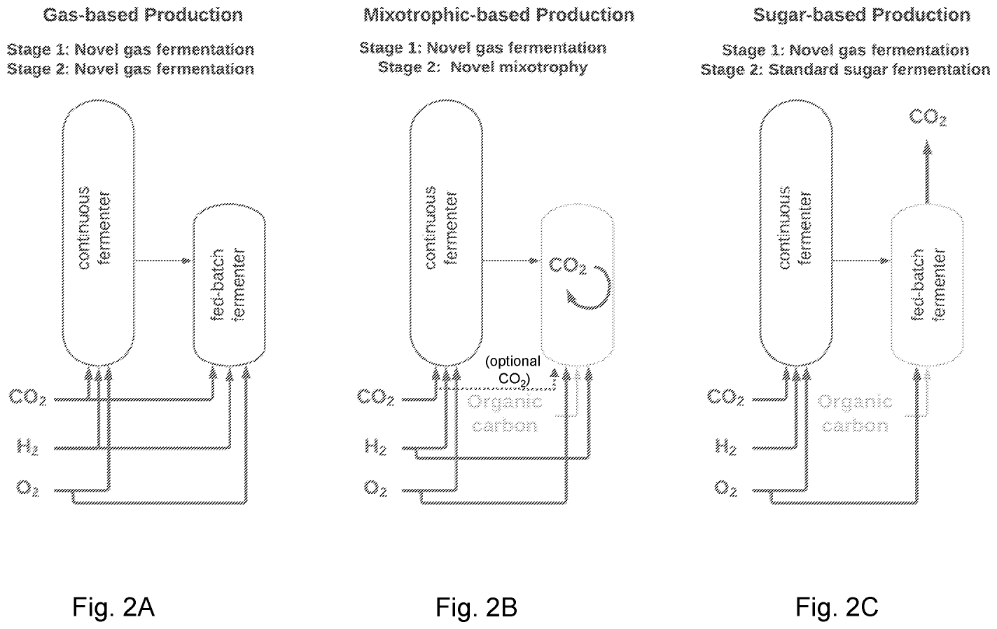

### DETAILED DESCRIPTION

Described herein are bioreactor systems and methods for producing a bioproduct from a microorganism (e.g., bacterium). Such systems and methods use microorganisms (e.g., bacteria) that are capable of both organic carbon fermentation and gas fermentation, commonly referred to as mixotrophs. Importantly, such microorganisms (e.g., bacteria) are also capable of switching between organic carbon fermentation and gas fermentation, referred to herein as switchotrophs. The bioreactors described herein in some aspects can comprise at least one reactor chamber that induces gas fermentation and at least one reactor chamber that induces carbon fermentation. An exemplary system is a hybrid of continuous gas fermentation (H₂/O₂/CO₂) for biomass production and subsequent fed-batch mixotrophic fermentation (sugar and H₂).

The systems and methods described herein exhibit at least the following benefits compared to other bioproduction platforms: (1) gas feedstocks are more cost-effective, less land-intensive, have fewer restrictions to delivery in large volumes, and have smaller carbon footprints compared to carbohydrate-based feedstocks: (2) gas fermentation provides an austere environment unfavorable to contamination by other microorganisms: (3) the gas fermentation minimizes genetic drift since it is used solely to produce biomass: (4) the mixotrophic fermentation can use hydrogen to draw down any released CO₂ from growth on sugar, thus optimizing production of the bioproduct and minimizing CO₂ output: (5) the mixotrophic fermentation can minimize genetic drift since it is optimized for bioproduct product, not microbial (e.g., bacterial) growth: (6) the system is capable of producing a wide range of bioproducts; and/or (7) this approach addresses the limitations that other technologies face in feedstocks, productivity, and product tailoring, thus unlocking increased scale, improved economics, and meaningful sustainability.

Described herein are systems comprising at least one bacterium (e.g., engineered to produce a bioproduct or naturally producing a bioproduct). Non-limiting examples of bioproducts include polypeptides, glycoproteins, lipoproteins, lipids, monosaccharides, polysaccharides, nucleic acids, small molecules, or metabolites.

In one aspect, described herein is a system for producing a bioproduct comprising: at least one reactor chamber containing therein at least one solution selected from: (a) at least one growth solution comprising: (i) carbon dioxide (CO₂), hydrogen (H₂), and oxygen (O₂); or (ii) an organic carbon source, hydrogen (H₂), and oxygen (O₂), and optionally carbon dioxide (CO₂); or (b) at least one production solution comprising: (i) carbon dioxide (CO₂), hydrogen (H₂), and oxygen (O₂); (ii) an organic carbon source, hydrogen (H₂), and oxygen (O₂), and optionally carbon dioxide (CO₂); or (iii) an organic carbon source and oxygen (O₂); and wherein the at least one reactor chamber contains therein: at least one bacterium in the at least one growth solution and/or at least one production solution, wherein the at least one bacterium produces the bioproduct.

In one aspect, described herein is a system for producing a bioproduct comprising: at least one reactor chamber containing therein: (a) at least one growth solution comprising: (i) carbon dioxide (CO₂), hydrogen (H₂), and oxygen (O₂); or (ii) an organic carbon source, hydrogen (H₂), and oxygen (O₂), and optionally carbon dioxide (CO₂); (b) at least one production solution comprising: (i) carbon dioxide (CO₂), hydrogen (H₂), and oxygen (O₂); (ii) an organic carbon source, hydrogen (H₂), and oxygen (O₂), and optionally carbon dioxide (CO₂); or (iii) an organic carbon source and oxygen (O₂); and (c) at least one bacterium in the at least one growth solution and/or at least one production solution, wherein the at least one bacterium produces the bioproduct.

In some embodiments of any of the aspects, the system comprises one reactor chamber (i.e., a single reactor chamber). In some embodiments of any of the aspects, the system comprises at least two reactor chambers, e.g., at least one primary reactor chamber (e.g., comprising at least one growth solution) and at least one secondary reactor chamber (e.g., comprising at least one production solution). In some embodiments of any of the aspects, the system comprises 1, 2, 3, 4 5, 6, 7, 8, 9, 10 or more reactor chambers.

In some embodiments of any of the aspects, the system comprises one primary reactor chamber; a “primary reactor chamber” can also be referred to herein as a “growth reactor chamber.”

In some embodiments of any of the aspects, the system comprises at least one primary reactor chamber, e.g., 2, 3, 4, 5, 6, 7, 8, 9, 10 or more primary reactor chambers. The multiple primary reactor chambers can be connected to each other or they can be discontinuous from each other. Each primary reactor chamber can be connected to at least one other primary reactor chamber. Each primary reactor chamber can be connected to at least one secondary reactor chamber.

In some embodiments of any of the aspects, the system comprises one secondary reactor chamber; a “secondary reactor chamber” can also be referred to herein as a “production reactor chamber.” In some embodiments of any of the aspects, the system comprises at least one secondary reactor chamber, e.g., 2, 3, 4, 5, 6, 7, 8, 9, 10 or more secondary reactor chambers. The multiple secondary reactor chambers can be connected to each other or they can be discontinuous from each other. Each secondary reactor chamber can be connected to at least one other secondary reactor chamber. Each secondary reactor chamber can be connected to at least one primary reactor chamber. In some embodiments of any of the aspects, the system comprises one primary reactor chamber that is connected to each of two or three secondary reactor chambers (see e.g., Table 4).

In one aspect, described herein is a system for producing a bioproduct comprising: (a) a primary reactor chamber with at least one growth solution contained therein, wherein the at least one growth solution comprises: (i) carbon dioxide (CO₂), hydrogen (H₂), and oxygen (O₂), or (ii) an organic carbon source, hydrogen (H₂), and oxygen (O₂), and optionally carbon dioxide (CO₂); (b) at least one secondary reactor chamber with at least one production solution contained therein, wherein the at least one production solution comprises: (i) carbon dioxide (CO₂), hydrogen (H₂), and oxygen (O₂); (ii) an organic carbon source, hydrogen (H₂), and oxygen (O₂), and optionally carbon dioxide (CO₂); or (iii) an organic carbon source and oxygen (O₂); and (c) at least one bacterium in the at least one growth solution of the primary reactor chamber and/or at least one production solution of the secondary reactor chamber, wherein the at least one bacterium produces the bioproduct.

In one aspect, described herein is a system for producing a bioproduct comprising: (a) at least one reactor chamber (e.g., a single reactor chamber or a primary reactor chamber) with a first solution (also referred to herein as a “growth solution”) contained therein, wherein the first solution (e.g., growth solution) comprises (i) carbon dioxide (CO₂), hydrogen (H₂), and oxygen (O₂), or (ii) an organic carbon source, hydrogen (H₂), and oxygen (O₂), and optionally carbon dioxide (CO₂); and (b) at least one reactor chamber (e.g., the single reactor chamber or at least one secondary reactor chamber) with a second solution (also referred to herein as a “production solution”) contained therein, wherein the second solution (e.g., at least one production solution) comprises: (i) carbon dioxide (CO₂), hydrogen (H₂), and oxygen (O₂); (ii) an organic carbon source, hydrogen (H₂), and oxygen (O₂) and optionally carbon dioxide (CO₂); or (iii) an organic carbon source and oxygen (O₂).

In one aspect, described herein is at least one growth solution (e.g., in at least one reactor chamber or in a primary reactor chamber, e.g., with a first solution contained therein). In some embodiments of any of the aspects, the growth (e.g., first) solution comprises carbon dioxide (CO₂), hydrogen (H₂), and oxygen (O₂). In some embodiments of any of the aspects, the growth solution comprises an organic carbon source, hydrogen (H₂), and oxygen (O₂), and optionally carbon dioxide (CO₂). In some embodiments of any of the aspects, the growth solution comprises organic carbon source, hydrogen (H₂), and oxygen (O₂). In some embodiments of any of the aspects, the growth solution comprises organic carbon source, hydrogen (H₂), oxygen (O₂), and carbon dioxide (CO₂). Without wishing to be bound by theory, the inclusion of carbon dioxide (CO₂) in the growth solution and/or production solution can increase the growth rate and/or bioproduct production of the bacterium.

In some embodiments of any of the aspects, the system comprises a first growth solution and a second growth solution. In some embodiments of any of the aspects, the first and/or second growth solution comprises carbon dioxide (CO₂), hydrogen (H₂), and oxygen (O₂). In some embodiments of any of the aspects, the first and/or second growth solution comprises an organic carbon source, hydrogen (H₂), and oxygen (O₂), and optionally carbon dioxide (CO₂). In some embodiments of any of the aspects, the system comprises 1, 2, 3, 4 5, 6, 7, 8, 9, 10 or more growth solutions.

In one aspect, described herein is at least one production solution (e.g., in at least one reactor chamber or in a secondary reactor chamber, e.g., with a second solution contained therein). In some embodiments of any of the aspects, the production solution (e.g., second solution) comprises: (i) carbon dioxide (CO₂), hydrogen (H₂), and oxygen (O₂); (ii) an organic carbon source, hydrogen (H₂), and oxygen (O₂) and optionally carbon dioxide (CO₂); or (iii) an organic carbon source and oxygen (O₂). In one aspect, described herein is at least one reactor chamber (e.g., at least one reactor chamber or a secondary reactor chamber) with a production solution (e.g., second solution) contained therein. In some embodiments of any of the aspects, production solution comprises: carbon dioxide (CO₂), hydrogen (H₂), and oxygen (O₂). In some embodiments of any of the aspects, the production (e.g., second) solution comprises: an organic carbon source, hydrogen (H₂), and oxygen (O₂) and optionally carbon dioxide (CO₂). In one aspect, described herein is a secondary reactor chamber with a production solution (e.g., second solution) contained therein, wherein the production (e.g., second) solution comprises: an organic carbon source and oxygen (O₂).

In some embodiments of any of the aspects, the system comprises a first production solution and a second production solution. In some embodiments of any of the aspects, the first and/or second production solution comprises carbon dioxide (CO₂), hydrogen (H₂), and oxygen (O₂). In some embodiments of any of the aspects, the first and/or second growth production comprises an organic carbon source, hydrogen (H₂), and oxygen (O₂), and optionally carbon dioxide (CO₂). In some embodiments of any of the aspects, the first and/or second growth production comprises an organic carbon source and oxygen (O₂). In some embodiments of any of the aspects, the system comprises 1, 2, 3, 4 5, 6, 7, 8, 9, 10 or more production solutions.

In some embodiments of any of the aspects, the system further comprises at least one bacterium in the at least one growth solution in the at least one reactor chamber (e.g., first solution of the primary reactor chamber) and/or at least one production solution in the at least one reactor chamber (e.g., second solution of the secondary reactor chamber). In some embodiments of any of the aspects, the at least one bacterium produces a bioproduct (e.g., in at least one reactor chamber or in the at least one secondary reactor chamber). In some embodiments of any of the aspects, the at least one bacterium is in the at least one growth solution in the at least one reactor chamber (e.g., in at least one reactor chamber or in the first solution of the primary reactor chamber). In some embodiments of any of the aspects, the at least one bacterium is in the production solution in the at least one reactor chamber (e.g., in at least one reactor chamber or in the second solution of the at least one secondary reactor chamber). In some embodiments of any of the aspects, the at least one bacterium is in the at least one growth solution and the at least one production solution in the at least one reactor chamber (e.g., in at least one reactor chamber or in the first solution of the primary reactor chamber and in the second solution of the secondary reactor chamber).

In one aspect, described herein is a system for producing a bioproduct comprising: (a) at least one reactor chamber (e.g., a single reactor chamber or a primary reactor chamber) with at least one growth solution (e.g., a first solution) contained therein, wherein the at least one growth solution (e.g., the first solution) comprises carbon dioxide (CO₂), hydrogen (H₂), and oxygen (O₂); and (b) at least one reactor chamber (e.g., the single reactor chamber or at least one secondary reactor chamber) with at least one production solution (e.g., a second solution) contained therein, wherein the at least one production solution (e.g., the second solution) comprises: carbon dioxide (CO₂), hydrogen (H₂), and oxygen (O₂).

In one aspect, described herein is a system for producing a bioproduct comprising: (a) at least one reactor chamber (e.g., a single reactor chamber or a primary reactor chamber) with at least one growth solution (e.g., a first solution) contained therein, wherein the at least one growth solution (e.g., the first solution) comprises carbon dioxide (CO₂), hydrogen (H₂), and oxygen (O₂); and (b) at least one reactor chamber (e.g., the single reactor chamber or at least one secondary reactor chamber) with at least one production solution (e.g., a second solution) contained therein, wherein the at least one production solution (e.g., the second solution) comprises an organic carbon source, hydrogen (H₂), and oxygen (O₂) and optionally carbon dioxide (CO₂).

In one aspect, described herein is a system for producing a bioproduct comprising: (a) at least one reactor chamber (e.g., a single reactor chamber or a primary reactor chamber) with at least one growth solution (e.g., a first solution) contained therein, wherein the at least one growth solution (e.g., the first solution) comprises carbon dioxide (CO₂), hydrogen (H₂), and oxygen (O₂); and (b) at least one reactor chamber (e.g., the single reactor chamber or at least one secondary reactor chamber) with at least one production solution (e.g., a second solution) contained therein, wherein the at least one production solution (e.g., the second solution) comprises an organic carbon source and oxygen (O₂).

In one aspect, described herein is a system for producing a bioproduct comprising: (a) at least one reactor chamber (e.g., a single reactor chamber or a primary reactor chamber) with at least one growth solution (e.g., a first solution) contained therein, wherein the at least one growth solution (e.g., the first solution) comprises an organic carbon source, hydrogen (H₂), and oxygen (O₂), and optionally carbon dioxide (CO₂); and (b) at least one reactor chamber (e.g., the single reactor chamber or at least one secondary reactor chamber) with at least one production solution (e.g., a second solution) contained therein, wherein the at least one production solution (e.g., the second solution) comprises: carbon dioxide (CO₂), hydrogen (H₂), and oxygen (O₂).

In one aspect, described herein is a system for producing a bioproduct comprising: (a) at least one reactor chamber (e.g., a single reactor chamber or a primary reactor chamber) with at least one growth solution (e.g., a first solution) contained therein, wherein the at least one growth solution (e.g., the first solution) comprises an organic carbon source, hydrogen (H₂), and oxygen (O₂), and optionally carbon dioxide (CO₂); and (b) at least one reactor chamber (e.g., the single reactor chamber or at least one secondary reactor chamber) with at least one production solution (e.g., a second solution) contained therein, wherein the at least one production solution (e.g., the second solution) comprises an organic carbon source, hydrogen (H₂), and oxygen (O₂) and optionally carbon dioxide (CO₂).

In one aspect, described herein is a system for producing a bioproduct comprising: (a) at least one reactor chamber (e.g., a single reactor chamber or a primary reactor chamber) with at least one growth solution (e.g., a first solution) contained therein, wherein the at least one growth solution (e.g., the first solution) comprises an organic carbon source, hydrogen (H₂), and oxygen (O₂), and optionally carbon dioxide (CO₂); and (b) at least one reactor chamber (e.g., the single reactor chamber or at least one secondary reactor chamber) with at least one production solution (e.g., a second solution) contained therein, wherein the at least one production solution (e.g., the second solution) comprises an organic carbon source and oxygen (O₂).

In one aspect, described herein is a system for producing a bioproduct comprising: (a) at least one reactor chamber (e.g., a single reactor chamber or a primary reactor chamber) with at least one growth solution (e.g., a first solution) contained therein, wherein the at least one growth solution (e.g., the first solution) comprises carbon dioxide (CO₂), hydrogen (H₂), and oxygen (O₂); (b) at least one reactor chamber (e.g., the single reactor chamber or at least one secondary reactor chamber) with at least one production solution (e.g., a second solution) contained therein, wherein the at least one production solution (e.g., the second solution) comprises: carbon dioxide (CO₂), hydrogen (H₂), and oxygen (O₂); and (c) at least one bacterium in the at least one growth solution (e.g., the first solution) of the primary reactor chamber and/or the at least one production solution (e.g., second solution) of the secondary reactor chamber, wherein the at least one bacterium produces the bioproduct.

In one aspect, described herein is a system for producing a bioproduct comprising: (a) at least one reactor chamber (e.g., a single reactor chamber or a primary reactor chamber with at least one growth solution (e.g., a first solution) contained therein, wherein the at least one growth solution (e.g., the first solution) comprises carbon dioxide (CO₂), hydrogen (H₂), and oxygen (O₂); (b) at least one reactor chamber (e.g., the single reactor chamber or at least one secondary reactor chamber) with at least one production solution (e.g., a second solution) contained therein, wherein the at least one production solution (e.g., the second solution) comprises an organic carbon source, hydrogen (H₂), and oxygen (O₂) and optionally carbon dioxide (CO₂); and (c) at least one bacterium in the at least one growth solution (e.g., the first solution) of the primary reactor chamber and/or the at least one production solution (e.g., second solution) of the secondary reactor chamber, wherein the at least one bacterium produces the bioproduct.

In one aspect, described herein is a system for producing a bioproduct comprising: (a) at least one reactor chamber (e.g., a single reactor chamber or a primary reactor chamber with at least one growth solution (e.g., a first solution) contained therein, wherein the at least one growth solution (e.g., the first solution) comprises carbon dioxide (CO₂), hydrogen (H₂), and oxygen (O₂); (b) at least one reactor chamber (e.g., the single reactor chamber or at least one secondary reactor chamber with at least one production solution (e.g., a second solution) contained therein, wherein the at least one production solution (e.g., the second solution) comprises an organic carbon source and oxygen (O₂); and (c) at least one bacterium in the at least one growth solution (e.g., the first solution) of the primary reactor chamber and/or second solution of the secondary reactor chamber, wherein the at least one bacterium produces the bioproduct.

In one aspect, described herein is a system for producing a bioproduct comprising: (a) at least one reactor chamber (e.g., a single reactor chamber or a primary reactor chamber) with at least one growth solution (e.g., a first solution) contained therein, wherein the at least one growth solution (e.g., the first solution) comprises an organic carbon source, hydrogen (H₂), and oxygen (O₂), and optionally carbon dioxide (CO₂); (b) at least one reactor chamber (e.g., the single reactor chamber or at least one secondary reactor chamber) with at least one production solution (e.g., a second solution) contained therein, wherein the at least one production solution (e.g., the second solution) comprises: carbon dioxide (CO₂), hydrogen (H₂), and oxygen (O₂); and (c) at least one bacterium in the at least one growth solution (e.g., the first solution) of the primary reactor chamber and/or the at least one production solution (e.g., second solution) of the secondary reactor chamber, wherein the at least one bacterium produces the bioproduct.

In one aspect, described herein is a system for producing a bioproduct comprising: (a) at least one reactor chamber (e.g., a single reactor chamber or a primary reactor chamber with at least one growth solution (e.g., a first solution) contained therein, wherein the at least one growth solution (e.g., the first solution) comprises an organic carbon source, hydrogen (H₂), and oxygen (O₂), and optionally carbon dioxide (CO₂); (b) at least one reactor chamber (e.g., the single reactor chamber or at least one secondary reactor chamber) with at least one production solution (e.g., a second solution) contained therein, wherein the at least one production solution (e.g., the second solution) comprises an organic carbon source, hydrogen (H₂), and oxygen (O₂) and optionally carbon dioxide (CO₂); and (c) at least one bacterium in the at least one growth solution (e.g., the first solution) of the primary reactor chamber and/or the at least one production solution (e.g., second solution) of the secondary reactor chamber, wherein the at least one bacterium produces the bioproduct.

In one aspect, described herein is a system for producing a bioproduct comprising: (a) at least one reactor chamber (e.g., a single reactor chamber or a primary reactor chamber with at least one growth solution (e.g., a first solution) contained therein, wherein the at least one growth solution (e.g., the first solution) comprises an organic carbon source, hydrogen (H₂), and oxygen (O₂), and optionally carbon dioxide (CO₂); (b) at least one reactor chamber (e.g., the single reactor chamber or at least one secondary reactor chamber with at least one production solution (e.g., a second solution) contained therein, wherein the at least one production solution (e.g., the second solution) comprises an organic carbon source and oxygen (O₂); and (c) at least one bacterium in the at least one growth solution (e.g., the first solution) of the primary reactor chamber and/or second solution of the secondary reactor chamber, wherein the at least one bacterium produces the bioproduct.

In some embodiments of any of the aspects, the at least one reactor chamber (e.g., a single reactor chamber or a primary reactor chamber) is a continuous fermentation reactor chamber. As used herein, the term “continuous fermentation” refers to a microbial process with a constant flow of culture medium through the bioreactor. In some embodiments of any of the aspects, the at least one reactor chamber (e.g., a single reactor chamber or a primary reactor chamber) is a mixotrophic fermentation reactor chamber.

In some embodiments of any of the aspects, the at least one reactor chamber (e.g., a single reactor chamber, or primary reactor chamber and/or the secondary reactor chamber) is a gas fermentation reactor chamber. As used herein, the term “gas fermentation” refers to a microbial process by which gaseous feedstocks (such as carbon monoxide (CO), carbon dioxide (CO₂), hydrogen (H₂), syngas, methane (CH₄), biogas, etc.) are used as carbon and/or energy sources, and then converted into a bioproduct by the microorganisms. For example, gas-fermenting microorganisms can fix carbon dioxide (CO₂), e.g., into organic carbon. As another non-limiting example, gas-fermenting microorganisms can be autotrophs, chemolithotrophs, mixotrophs, or switchotrophs, as described further herein.

In some embodiments of any of the aspects, the at least one reactor chamber (e.g., a single reactor chamber or secondary reactor chamber) is an organic carbon fermentation reactor chamber. As used herein, the term “organic carbon fermentation” refers to a microbial process by which organic carbon sources (e.g., glucose, glycerol, gluconate, acetate, fructose, decanoate, etc.) are converted into a bioproduct by the microorganisms. As a non-limiting example, organic carbon-fermenting microorganisms can be heterotrophs, mixotrophs or switchotrophs, as described further herein. In some embodiments of any of the aspects, the organic carbon-fermenting microorganism is not a heterotroph.

In some embodiments of any of the aspects, the at least one reactor chamber (e.g., a single reactor chamber or secondary reactor chamber) is a mixotrophic fermentation reactor chamber. As used herein, the term “mixotrophic fermentation” refers to a microbial process by which both gas fermentation and organic carbon fermentation occur. In other words, gaseous feedstocks (such as carbon monoxide (CO), carbon dioxide (CO₂), hydrogen (H₂), syngas, methane (CH₄), biogas, etc.) are used as carbon and/or energy sources, and organic carbon sources (e.g., glucose, glycerol, gluconate, acetate, fructose, decanoate, etc.) are also used as carbon sources, and then the gaseous feedstocks and organic carbon sources are converted into a bioproduct by the microorganisms. Products, byproducts, metabolites, chemical, gases, etc., from gas fermentation can feed into organic carbon fermentation. Products, byproducts, metabolites, chemical, gases, etc., from organic carbon fermentation can feed into gas fermentation. As a non-limiting example, gases produced by organic carbon fermentation (e.g., carbon dioxide (CO₂)) can feed into gas fermentation. As a non-limiting example, mixotrophic-fermenting microorganisms can be mixotrophs or switchotrophs, as described further herein.

In some embodiments of any of the aspects, the at least one reactor chamber (e.g., a single reactor chamber or secondary reactor chamber) is a fed-batch fermentation reactor chamber. As used herein, the term “fed-batch fermentation” (or “batch-fed formation”) refers to a microbial process where one or more nutrients/solutions are fed into the bioreactor during culturing. In some embodiments of any of the aspects, the bioproduct(s) remain in the bioreactor until the end of the batch or run, e.g., until the bacterium produces a pre-determined concentration of the bioproduct. In some embodiments of any of the aspects, the at least one growth solution (e.g., the first solution) from at least one reactor chamber (e.g., the primary reactor chamber) is batch fed into at least one other reactor chamber (e.g., the secondary reactor chamber). In some embodiments of any of the aspects, the at least one growth solution (e.g., the first solution) from the at least one reactor chamber (e.g., primary reactor chamber) comprises a pre-determined concentration of bacterium in culture medium.

In some embodiments of any of the aspects, at least two reactor chambers (e.g., primary and secondary reactor chambers) are physically linked. As a non-limiting example the at least two reactor chambers (e.g., primary and secondary reactor chambers) are connected via pipes, tubes, tubing, or another connection, any one of which can comprise a valve or another fixture to control the flow of material between the reactor chambers. In some embodiments of any of the aspects, 1 growth reactor chamber (e.g., primary reactor chamber) is physically linked to 1, 2, 3, 4, 5, 6, 7, 8, 9, 10 or more production reactor chamber(s) (e.g., secondary reactor chamber(s)). In some embodiments of any of the aspects, 1, 2, 3, 4, 5, 6, 7, 8, 9, 10 or more growth reactor chamber(s) (e.g., primary reactor chamber(s)) are physically linked to 1 production reactor chamber (e.g., secondary reactor chamber). In some embodiments of any of the aspects, 1, 2, 3, 4, 5, 6, 7, 8, 9, 10 or more growth reactor chamber(s) (e.g., primary reactor chamber(s)) are physically linked to 1, 2, 3, 4, 5, 6, 7, 8, 9, 10 or more production reactor chamber(s) (e.g., secondary reactor chamber(s)).

In some embodiments of any of the aspects, the at least one reactor chamber (e.g., a single reactor chamber or secondary reactor chamber) is: (a) a gas fermentation reactor chamber; (b) a gas and organic carbon (mixotrophic) fermentation reactor chamber; or (c) an organic carbon fermentation reactor chamber. In some embodiments of any of the aspects, the at least one reactor chamber (e.g., a single reactor chamber or secondary reactor chamber) is a gas fermentation reactor chamber. In some embodiments of any of the aspects, the at least one reactor chamber (e.g., a single reactor chamber or secondary reactor chamber) is a gas and organic carbon (mixotrophic) fermentation reactor chamber. In some embodiments of any of the aspects, the at least one reactor chamber (e.g., a single reactor chamber or secondary reactor chamber) is: an organic carbon fermentation reactor chamber (see e.g., Table 4).

TABLE 4
Exemplary conditions for reactor steps or reactor chambers.

| Reactor Steps or | | | | Reactor Steps or | | | | Reactor Steps or | | | |
|---|---|---|---|---|---|---|---|---|---|---|---|
| Chambers | | | | Chambers | | | | Chambers | | | |
| | Production | | | | Production | | | | Production | | |
| Growth | 1st | 2nd | 3rd | Growth | 1st | 2nd | 3rd | Growth | 1st | 2nd | 3rd |
| gas | gas | | | gas | gas | gas | mix | gas | mix | mix | OC |
| gas | mix | | | gas | gas | gas | OC | gas | mix | OC | gas |
| gas | OC | | | gas | gas | mix | gas | gas | mix | OC | mix |
| gas | gas | gas | | gas | gas | mix | mix | gas | mix | OC | OC |
| gas | gas | mix | | gas | gas | mix | OC | gas | OC | gas | gas |
| gas | gas | OC | | gas | gas | OC | gas | gas | OC | gas | mix |
| gas | mix | gas | | gas | gas | OC | mix | gas | OC | gas | OC |
| gas | mix | mix | | gas | gas | OC | OC | gas | OC | mix | gas |
| gas | mix | OC | | gas | mix | gas | gas | gas | OC | mix | mix |
| gas | OC | gas | | gas | mix | gas | mix | gas | OC | mix | OC |
| gas | OC | mix | | gas | mix | gas | OC | gas | OC | OC | gas |
| gas | OC | OC | | gas | mix | mix | gas | gas | OC | OC | mix |
| gas | gas | gas | gas | gas | mix | mix | mix | gas | OC | OC | OC |
| mix | gas | | | mix | gas | gas | mix | mix | mix | mix | OC |
| mix | mix | | | mix | gas | gas | OC | mix | mix | OC | gas |
| mix | OC | | | mix | gas | mix | gas | mix | mix | OC | mix |
| mix | gas | gas | | mix | gas | mix | mix | mix | mix | OC | OC |
| mix | gas | mix | | mix | gas | mix | OC | mix | OC | gas | gas |
| mix | gas | OC | | mix | gas | OC | gas | mix | OC | gas | mix |
| mix | mix | gas | | mix | gas | OC | mix | mix | OC | gas | OC |
| mix | mix | mix | | mix | gas | OC | OC | mix | OC | mix | gas |
| mix | mix | OC | | mix | mix | gas | gas | mix | OC | mix | mix |
| mix | OC | gas | | mix | mix | gas | mix | mix | OC | mix | OC |
| mix | OC | mix | | mix | mix | gas | OC | mix | OC | OC | gas |
| mix | OC | OC | | mix | mix | mix | gas | mix | OC | OC | mix |
| mix | gas | gas | gas | mix | mix | mix | mix | mix | OC | OC | OC |

“Gas” indicates gas fermentation (e.g., CO₂, H₂, and O₂); “mix” indicates mixotrophic fermentation (e.g., organic carbon source, H₂, and O₂, optionally CO₂); “OC” indicates organ carbon fermentation (e.g., organic carbon source and O₂).

In some embodiments of any of the aspects, the system comprises at least one growth solution and at least one production solution (see e.g., Table 5). In some embodiments of any of the aspects, the system comprises one growth solution and one production solution. In some embodiments of any of the aspects, the system comprises one growth solution and two production solutions. In some embodiments of any of the aspects, the system comprises two growth solutions and one production solution. In some embodiments of any of the aspects, the system comprises two growth solutions and two production solutions. In some embodiments of any of the aspects, the first growth solution can be used for a pre-determined period of time, and then a second growth solution can be used. In some embodiments of any of the aspects, the first production solution can be used for a pre-determined period of time, and then a second production solution can be used.

TABLE 5
Exemplary growth and production solutions.

| Growth | | Production | | Growth | | Production | | Growth | | Production | |
|---|---|---|---|---|---|---|---|---|---|---|---|
| 1st | 2nd | 1st | 2nd | 1st | 2nd | 1st | 2nd | 1st | 2nd | 1st | 2nd |
| gas | | gas | | gas | | gas | mix | gas | gas | gas | OC |
| gas | | mix | | gas | | mix | mix | gas | gas | mix | OC |
| gas | | OC | | gas | | OC | mix | gas | gas | OC | OC |
| mix | | gas | | mix | | gas | mix | mix | gas | gas | OC |
| mix | | mix | | mix | | mix | mix | mix | gas | mix | OC |
| mix | | OC | | mix | | OC | mix | mix | gas | OC | OC |
| gas | gas | gas | | gas | | gas | OC | gas | mix | gas | gas |
| gas | gas | mix | | gas | | mix | OC | gas | mix | mix | gas |
| gas | gas | OC | | gas | | OC | OC | gas | mix | OC | gas |
| mix | gas | gas | | mix | | gas | OC | mix | mix | gas | gas |
| mix | gas | mix | | mix | | mix | OC | mix | mix | mix | gas |
| mix | gas | OC | | mix | | OC | OC | mix | mix | OC | gas |
| gas | mix | gas | | gas | gas | gas | gas | gas | mix | gas | mix |
| gas | mix | mix | | gas | gas | mix | gas | gas | mix | mix | mix |
| gas | mix | OC | | gas | gas | OC | gas | gas | mix | OC | mix |
| mix | mix | gas | | mix | gas | gas | gas | mix | mix | gas | mix |
| mix | mix | mix | | mix | gas | mix | gas | mix | mix | mix | mix |
| mix | mix | OC | | mix | gas | OC | gas | mix | mix | OC | mix |
| gas | | gas | gas | gas | gas | gas | mix | gas | mix | gas | OC |
| gas | | mix | gas | gas | gas | mix | mix | gas | mix | mix | OC |
| gas | | OC | gas | gas | gas | OC | mix | gas | mix | OC | OC |
| mix | | gas | gas | mix | gas | gas | mix | mix | mix | gas | OC |
| mix | | mix | gas | mix | gas | mix | mix | mix | mix | mix | OC |
| mix | | OC | gas | mix | gas | OC | mix | mix | mix | OC | OC |

“Gas” indicates gas fermentation (e.g., CO₂, H₂, and O₂); “mix” indicates mixotrophic fermentation (e.g., organic carbon source, H₂, and O₂, optionally CO₂); “OC” indicates organ carbon fermentation (e.g., organic carbon source and O₂).

In one aspect, the system comprises at least one bacteria and a support. In some embodiments of any of the aspects, the bacteria are linked to the support using intrinsic mechanisms (e.g., pili, biofilm, etc.) and/or extrinsic mechanisms (e.g., chemical crosslinking, antibiotics, opsonin, etc.). In some embodiments of any of the aspects, the system further comprises a container and a solution, in which the bacteria linked to the support are submerged.

In some embodiments of any of the aspects, the system further comprises a pair of electrodes that split water contained within the solution to form hydrogen. In some embodiments of any of the aspects, at least one reactor chamber (e.g. a single reactor chamber or the primary and/or secondary reactor chamber) further comprises a pair of electrodes in contact with the at least one growth and/or production solution (e.g., first and/or second solution) that split water to form the hydrogen. In some embodiments of any of the aspects, the at least one reactor chamber (e.g., a single reactor chamber or primary reactor chamber) further comprises a pair of electrodes in contact with the at least one growth solution (e.g., the first solution) that split water to form the hydrogen. In some embodiments of any of the aspects, the at least one reactor chamber (e.g., the single reactor chamber or secondary reactor chamber) further comprises a pair of electrodes in contact with the at least one production solution (e.g., the second solution) that split water to form the hydrogen. In some embodiments of any of the aspects, the at least one reactor chamber (e.g., a single reactor chamber, or primary and secondary reactor chambers) further comprise a pair of electrodes in contact with the at least one growth and/or production solution (e.g., first and second solution) that split water to form the hydrogen.

In some embodiments of any of the aspects, the solution (e.g., a culture medium) comprises hydrogen (H₂) and carbon dioxide (CO₂). In some embodiments of any of the aspects, the gasses in the solution (e.g., a culture medium) consist of hydrogen (H₂) and carbon dioxide (CO₂). In some embodiments of any of the aspects, the gasses in the solution (e.g., a culture medium) consist of oxygen (O₂) and carbon dioxide (CO₂). In some embodiments of any of the aspects, the gasses in the solution (e.g., a culture medium) consist of hydrogen (H₂) and oxygen (O₂). In some embodiments of any of the aspects, the solution (e.g., a culture medium) comprises carbon dioxide (CO₂), hydrogen (H₂), and oxygen (O₂). In some embodiments of any of the aspects, the gasses in the solution (e.g., a culture medium) consist of carbon dioxide (CO₂), hydrogen (H₂), and oxygen (O₂). In some embodiments of any of the aspects, the gasses in the solution (e.g., a culture medium) consist of carbon dioxide (CO₂). In some embodiments of any of the aspects, the gasses in the solution (e.g., a culture medium) consist of hydrogen (H₂). In some embodiments of any of the aspects, the gasses in the solution (e.g., a culture medium) consist of oxygen (O₂). In some embodiments of any of the aspects, the solution (e.g., a culture medium) comprises an organic carbon source, hydrogen (H₂), and oxygen (O₂) and optionally carbon dioxide (CO₂). In some embodiments of any of the aspects, the solution (e.g., a culture medium) comprises an organic carbon source and oxygen (O₂). In some embodiments of any of the aspects, the solution (e.g., a culture medium) comprises hydrogen (H₂) and glycerol. In some embodiments of any of the aspects, the solution (e.g., a culture medium) comprises hydrogen (H₂), glycerol, and carbon dioxide (CO₂).

In some embodiments of any of the aspects, the support comprises a solid substrate. Examples of solid substrate can include, but are not limited to, film, beads or particles (including nanoparticles, microparticles, polymer microbeads, magnetic microbeads, and the like), filters, fibers, screens, mesh, tubes, hollow fibers, scaffolds, plates, channels, gold particles, magnetic materials, medical apparatuses (e.g., needles or catheters) or implants, dipsticks or test strips, filtration devices or membranes, hollow fiber cartridges, microfluidic devices, mixing elements (e.g., spiral mixers), extracorporeal devices, and other substrates commonly utilized in assay formats, and any combinations thereof. In some embodiments of any of the aspects, the solid substrate can be a magnetic particle or bead.

In several aspects, the system comprises primary and/or secondary reactor chambers and at least one of the bacteria as described herein. Accordingly, in one aspect, described herein is a system comprising: (a) at least one reactor chamber (e.g., a single reactor chamber or a primary reactor chamber) with a solution contained therein, wherein the solution comprises oxygen (O₂), hydrogen (H₂) and carbon dioxide (CO₂); and (b) at least one bacterium in the solution. Also described herein is a system comprising: (a) at least one reactor chamber (e.g., a single reactor chamber or a secondary reactor chamber) with a solution contained therein, wherein the solution comprises: (i) carbon dioxide (CO₂), hydrogen (H₂), and oxygen (O₂); (ii) an organic carbon source, hydrogen (H₂), and oxygen (O₂) and optionally carbon dioxide (CO₂); or (iii) an organic carbon source and oxygen (O₂); and (b) at least one bacterium in the solution. In some embodiments of any of the aspects, the system further comprises a pair of electrodes in contact with the solution that split water to form the hydrogen. In one aspect, described herein is a system comprising: (a) at least one reactor chamber (e.g., a single reactor chamber or a primary reactor chamber); and (b) at least one bacterium. In one aspect, described herein is a system comprising: (a) at least one reactor chamber (e.g., a single reactor chamber or a secondary reactor chamber); and (b) at least one bacterium. In one aspect, described herein is a system comprising: (a) a primary reactor chamber; (b) at least one secondary reactor chamber; and (c) at least one bacterium. In one aspect, described herein is a system comprising: (a) at least one reactor chamber; and (b) at least one bacterium. In some embodiments of any of the aspects, the system further comprises a pair of electrodes in contact with the at least one reactor chamber. In some embodiments of any of the aspects, the system (e.g., a system comprising at least one reactor chamber, a system comprising a support) can comprise any combination of bacteria.

In one aspect, described herein is a system comprising: (a) at least one reactor chamber (e.g., a single reactor chamber or a primary reactor chamber) with at least one growth solution (e.g., a first solution) contained therein, wherein the at least one growth solution (e.g., the first solution) comprises: (i) carbon dioxide (CO₂), hydrogen (H₂), and oxygen (O₂), or (ii) an organic carbon source, hydrogen (H₂), and oxygen (O₂), and optionally carbon dioxide (CO₂); (b) at least one reactor chamber (e.g., the single reactor chamber or a secondary reactor chamber) with at least one production solution (e.g., a second solution) contained therein, wherein the at least one production solution (e.g., the second solution) comprises: (i) carbon dioxide (CO₂), hydrogen (H₂), and oxygen (O₂); (ii) an organic carbon source, hydrogen (H₂), and oxygen (O₂) and optionally carbon dioxide (CO₂); or (iii) an organic carbon source and oxygen (O₂); (c) a bacterium in the solution in the at least one reactor chamber (e.g., a single reactor chamber or primary and/or secondary reactor chamber(s)); and (d) a pair of electrodes in contact with the solution in the at least one reactor chamber (e.g., the single reactor chamber or primary and/or secondary reactor chamber(s)) that split water to form the hydrogen.

In one aspect, described herein is a system comprising: (a) at least one reactor chamber (e.g., a single reactor chamber or a primary reactor chamber) with at least one growth solution (e.g., a first solution) contained therein, wherein the at least one growth solution (e.g., the first solution) comprises carbon dioxide (CO₂), hydrogen (H₂), and oxygen (O₂); (b) at least one reactor chamber (e.g., the single reactor chamber or a secondary reactor chamber) with at least one production solution (e.g., a second solution) contained therein, wherein the at least one production solution (e.g., the second solution) comprises: carbon dioxide (CO₂), hydrogen (H₂), and oxygen (O₂); (c) a bacterium in the solution in the at least one reactor chamber (e.g., the single reactor chamber or primary and/or secondary reactor chamber(s)); and (d) a pair of electrodes in contact with the solution in the at least one reactor chamber (e.g., the single reactor chamber or primary and/or secondary reactor chamber(s)) that split water to form the hydrogen.

In one aspect, described herein is a system comprising: (a) at least one reactor chamber (e.g., a single reactor chamber or a primary reactor chamber) with at least one growth solution (e.g., a first solution) contained therein, wherein the at least one growth solution (e.g., the first solution) comprises carbon dioxide (CO₂), hydrogen (H₂), and oxygen (O₂); (b) at least one reactor chamber (e.g., the single reactor chamber or a secondary reactor chamber) with at least one production solution (e.g., a second solution) contained therein, wherein the at least one production solution (e.g., the second solution) comprises: an organic carbon source, hydrogen (H₂), and oxygen (O₂) and optionally carbon dioxide (CO₂); (c) a bacterium in the solution in the at least one reactor chamber (e.g., the single reactor chamber or primary and/or secondary reactor chamber(s)); and (d) a pair of electrodes in contact with the solution in the at least one reactor chamber (e.g., the single reactor chamber or primary and/or secondary reactor chamber(s)) that split water to form the hydrogen.

In one aspect, described herein is a system comprising: (a) at least one reactor chamber (e.g., a single reactor chamber or a primary reactor chamber) with at least one growth solution (e.g., a first solution) contained therein, wherein the at least one growth solution (e.g., the first solution) comprises carbon dioxide (CO₂), hydrogen (H₂), and oxygen (O₂); (b) at least one reactor chamber (e.g., the single reactor chamber or a secondary reactor chamber) with at least one production solution (e.g., a second solution) contained therein, wherein the at least one production solution (e.g., the second solution) comprises: an organic carbon source and oxygen (O₂); (c) a bacterium in the solution in the at least one reactor chamber (e.g., the single reactor chamber or primary and/or secondary reactor chamber(s)); and (d) a pair of electrodes in contact with the solution in the at least one reactor chamber (e.g., the single reactor chamber or primary and/or secondary reactor chamber(s)) that split water to form the hydrogen.

In one aspect, described herein is a system comprising: (a) at least one reactor chamber (e.g., a single reactor chamber or a primary reactor chamber) with at least one growth solution (e.g., a first solution) contained therein, wherein the at least one growth solution (e.g., the first solution) comprises an organic carbon source, hydrogen (H₂), and oxygen (O₂), and optionally carbon dioxide (CO₂); (b) at least one reactor chamber (e.g., the single reactor chamber or a secondary reactor chamber) with at least one production solution (e.g., a second solution) contained therein, wherein the at least one production solution (e.g., the second solution) comprises: carbon dioxide (CO₂), hydrogen (H₂), and oxygen (O₂); (c) a bacterium in the solution in the at least one reactor chamber (e.g., the single reactor chamber or primary and/or secondary reactor chamber(s)); and (d) a pair of electrodes in contact with the solution in the at least one reactor chamber (e.g., the single reactor chamber or primary and/or secondary reactor chamber(s)) that split water to form the hydrogen.

In one aspect, described herein is a system comprising: (a) at least one reactor chamber (e.g., a single reactor chamber or a primary reactor chamber) with at least one growth solution (e.g., a first solution) contained therein, wherein the at least one growth solution (e.g., the first solution) comprises an organic carbon source, hydrogen (H₂), and oxygen (O₂), and optionally carbon dioxide (CO₂); (b) at least one reactor chamber (e.g., the single reactor chamber or a secondary reactor chamber) with at least one production solution (e.g., a second solution) contained therein, wherein the at least one production solution (e.g., the second solution) comprises: an organic carbon source, hydrogen (H₂), and oxygen (O₂) and optionally carbon dioxide (CO₂); (c) a bacterium in the solution in the at least one reactor chamber (e.g., the single reactor chamber or primary and/or secondary reactor chamber(s)); and (d) a pair of electrodes in contact with the solution in the at least one reactor chamber (e.g., the single reactor chamber or primary and/or secondary reactor chamber(s)) that split water to form the hydrogen.

In one aspect, described herein is a system comprising: (a) at least one reactor chamber (e.g., a single reactor chamber or a primary reactor chamber) with at least one growth solution (e.g., a first solution) contained therein, wherein the at least one growth solution (e.g., the first solution) comprises an organic carbon source, hydrogen (H₂), and oxygen (O₂), and optionally carbon dioxide (CO₂); (b) at least one reactor chamber (e.g., the single reactor chamber or a secondary reactor chamber) with at least one production solution (e.g., a second solution) contained therein, wherein the at least one production solution (e.g., the second solution) comprises: an organic carbon source and oxygen (O₂); (c) a bacterium in the solution in the at least one reactor chamber (e.g., the single reactor chamber or primary and/or secondary reactor chamber(s)); and (d) a pair of electrodes in contact with the solution in the at least one reactor chamber (e.g., the single reactor chamber or primary and/or secondary reactor chamber(s)) that split water to form the hydrogen.

In some embodiments of any of the aspects, the pair of electrodes comprise a cathode including a cobalt-phosphorus alloy and an anode including cobalt phosphate.

In some embodiments of any of the aspects, the system further comprises at least one (e.g., 1, 2, 3 4, 5, 6, 7, 8, 9, 10, or more) an inducer solution(s). In some embodiments of any of the aspects, the system comprises one inducer solution. In some embodiments of any of the aspects, the inducer solution is used to induce the bacterium to produce at least one bioproduct in the at least one reactor chamber (e.g., a single reactor chamber or a secondary reactor chamber). In some embodiments of any of the aspects, the inducer solution is used after the at least one growth solution. In some embodiments of any of the aspects, the inducer solution is used before the at least one production solution.

In some embodiments of any of the aspects, the inducer induces expression of the bioproduct from an inducible promoter. Non-limiting examples of inducible promoters include: a doxycycline-inducible promoter, the lac promoter, the lacUV5 promoter, the tac promoter, the trc promoter, the T5 promoter, the T7 promoter, the T7-lac promoter, the araBAD promoter, the rha promoter, the tet promoter, an isopropyl β-D-1-thiogalactopyranoside (IPTG)-dependent promoter, an AlcA promoter, a LexA promoter, a temperature inducible promoter (e.g., Hsp70 or Hsp90-derived promoters), or a light inducible promoter (e.g., pDawn/YFI/FixK2 promoter/CI/pR promoter system).

In some embodiments of any of the aspects, the inducer is arabinose and the bioproduct is encoded in an arabinose-inducible vector or under the control of an arabinose-inducible promoter (e.g., pBAD).

In some embodiments of any of the aspects, a concentration of the bioavailable nitrogen in the inducer solution is below a threshold nitrogen concentration to induce or cause the bacteria to produce a product. In some embodiments of any of the aspects, an external inducer can be used to induce production of the product. Non-limiting examples of an external inducer include: isopropyl β-D-1-thiogalactopyranoside (IPTG), glucose, arabinose, anhydrotetracycline, rhamnose, or xylose. In some embodiments of any of the aspects, the inducer solution is also referred to as a culture medium and can comprise a minimal medium or a defined medium as described further herein.

In some embodiments of any of the aspects, the inducer solution further comprises: carbon dioxide (CO₂), hydrogen (H₂), and oxygen (O₂). In some embodiments of any of the aspects, the inducer solution further comprises: an organic carbon source, hydrogen (H₂), and oxygen (O₂), and optionally carbon dioxide (CO₂). In some embodiments of any of the aspects, the inducer solution further comprises: an organic carbon source and oxygen (O₂).

In one embodiment, a system includes at least one reactor chamber (e.g., a single reactor chamber) containing a solution. In one embodiment, a system includes a primary and/or secondary reactor chamber containing a solution. The solution may include hydrogen (H₂), carbon dioxide (CO₂), oxygen (O₂), bioavailable nitrogen, and a bacterium. Gasses such as one or more of hydrogen (H₂), carbon dioxide (CO₂), nitrogen (N₂), and oxygen (O₂) may also be located within a headspace of the reactor chamber, though embodiments in which a reactor does not include a headspace such as in a flow through reactor are also contemplated. The system may also include a pair of electrodes immersed in the solution. The electrodes are configured to apply a voltage potential to, and pass a current through, the solution to split water contained within the solution to form at least hydrogen (H₂) and oxygen (O₂) gasses in the solution. These gases may then become dissolved in the solution. During use, a concentration of the bioavailable nitrogen in the solution may be maintained below a threshold nitrogen concentration that causes the bacteria to produce a desired product. This product may either by excreted from the bacteria and/or stored within the bacteria as the disclosure is not so limited.

Concentrations of the above noted gases both dissolved within a solution, and/or within a headspace above the solution, may be controlled in any number of ways including bubbling gases through the solution, generating the dissolved gases within the solution as noted above (e.g. electrolysis/water splitting), periodically refreshing a composition of gases located within a headspace above the solution, or any other appropriate method of controlling the concentration of dissolved gas within the solution. In some embodiments of any of the aspects, the gases are flowed (at one timepoint or multiple timepoints) into the reactor chamber. In some embodiments of any of the aspects, gases are added to the primary and/or secondary reactor chamber(s) prior to cultivation or culturing of the microorganisms. In some embodiments of any of the aspects, the gases are mixed prior to inflow into the reactor chamber. In some embodiments of any of the aspects, the gases are constantly sparged into the solution. Additionally, the various methods of controlling concentration may either be operated in a steady-state mode with constant operating parameters, and/or a concentration of one or more of the dissolved gases may be monitored to enable a feedback process to actively change the concentrations, generation rates, or other appropriate parameter to change the concentration of dissolved gases to be within the desired ranges noted herein. Monitoring of the gas concentrations may be done in any appropriate manner including pH monitoring, dissolved oxygen meters, gas chromatography, or any other appropriate method.

As noted above, in one embodiment, the composition of a volume of gas located in a headspace of a reactor may include one or more of carbon dioxide, oxygen, hydrogen, and nitrogen. A concentration of the carbon dioxide may be between 10 volume percent (vol %) and 100 vol %. However, carbon dioxide may also be greater than equal to 0.04 vol % and/or any other appropriate concentration. For example, carbon dioxide may be between or equal to 0.04 vol % and 100 vol %. In some embodiments, a concentration of carbon dioxide may be between or equal to 0.04 vol % and 50 vol %. In some embodiments, a concentration of the oxygen may be between 0 vol % and 100 vol % and/or any other appropriate concentration. In some embodiments, a concentration of the oxygen may be between 1 vol % and 99 vol % and/or any other appropriate concentration. In some embodiments, a concentration of the oxygen may be between 0.05 vol % and 50 vol % and/or any other appropriate concentration. A concentration of the hydrogen may be greater than or equal to 0.05 vol % and 99 vol %. A concentration of the nitrogen may be between 0 vol % and 99 vol %.

As also noted, in one embodiment, a solution within a reactor chamber may include water as well as one or more of carbon dioxide, oxygen, and hydrogen dissolved within the water. A concentration of the carbon dioxide in the solution may be between 0.04 vol % to saturation within the solution. A concentration of the oxygen in the solution may be between 1 vol % to saturation within the solution. A concentration of the hydrogen in the solution may be between 0.05 vol % to saturation within the solution provided that appropriate concentrations of carbon dioxide and/or oxygen are also present.

As noted previously, and as described further below, production of a desired end product by bacteria located within the solution may be controlled by limiting a concentration of bioavailable nitrogen, such as in the form of ammonia, amino acids, or any other appropriate source of nitrogen useable by the bacteria within the solution to below a threshold nitrogen concentration. However, and without wishing to be bound by theory, the concentration threshold may be different for different bacteria and/or for different concentrations of bacteria. For example, a solution containing enough ammonia to support a Ralstonia eutropha (i.e., Cupriavidus necator) population up to an optical density (OD) of 2.3 produces product at molar concentrations less than or equal to 0.03 M while a population with an OD of 0.7 produces product at molar concentrations less than or equal to 0.9 mM. Accordingly, higher optical densities may be correlated with producing product at higher nitrogen concentrations while lower optical densities may be correlated with producing product at lower nitrogen concentrations. Further, bacteria may be used to produce product by simply placing them in solutions containing no nitrogen. In view of the above, an optical density of bacteria within a solution may be between or equal to 0.1 and 12, 0.7 and 12, or any other appropriate concentration including concentrations both larger and smaller than those noted above. Additionally, a concentration of nitrogen within the solution may be between or equal to 0 molar and 0.2 molar, 0.0001 molar and 0.1 molar, 0.0001 molar and 0.05 molar, 0.0001 molar and 0.03 molar, or any other appropriate composition including compositions greater and less than the ranges noted above.

Bacteria used in the systems and methods disclosed herein may be selected so that the bacteria both oxidize hydrogen as well as consume carbon dioxide. Accordingly, in some embodiments, the bacteria may include an enzyme capable of metabolizing hydrogen as an energy source such as with hydrogenase enzymes. Additionally, the bacteria may include one or more enzymes capable of performing carbon fixation such as Ribulose-1,5-bisphosphate carboxy lase/oxygenase (RuBisCO). One possible class of bacteria that may be used in the systems and methods described herein to produce a product include, but are not limited to, chemolithoautotrophs. Additionally, appropriate chemolithoautotrophs may include any one or more of Ralstonia eutropha (R. eutropha) as well as Alcaligenes paradoxs I 360 bacteria, Alcaligenes paradoxs 12/X bacteria, Nocardia opaca bacteria, Nocardia autotrophica bacteria, Paracoccus denitrificans bacteria, Pseudomonas facilis bacteria, Arthrobacter species 11× bacteria, Xanthobacter autotrophicus bacteria, Azospirillum lipferum bacteria, Derxia Gummosa bacteria, Rhizobium japonicum bacteria, Microcyclus aquaticus bacteria, Microcyclus ebruneus bacteria, Renobacter vacuolatum bacteria, and any other appropriate bacteria.

A bacterium in the system or bioreactor can either naturally include a bioproduct production pathway, or may be appropriately engineered, to include a bioproduct production pathway when placed under the appropriate growth conditions.

One embodiment of a system can include one or more reactor chambers (see e.g., US Patent Publication 2018/0265898, which is incorporated herein by reference in its entirety). In some embodiments, a reactor chamber houses one or more pairs of electrodes including an anode and a cathode immersed in a water based solution. Bacteria are also included in the solution. The reactor chamber can be at least one reactor chamber, a primary reactor chamber, or a secondary reactor chamber. The reactor chamber can be physically linked to another reactor chamber, such as a primary or a secondary reactor chamber. A headspace corresponding to a volume of gas that is isolated from an exterior environment is located above the solution within the reactor chamber. The gas volume may correspond to any appropriate composition including, but not limited to, carbon dioxide, nitrogen, hydrogen, oxygen, and any other appropriate gases as the disclosure is not so limited. Additionally, as detailed further below, the various gases may be present in any appropriate concentration as detailed previously. However, it should be understood that embodiments in which a reactor chamber is exposed to an external atmosphere that may either be a controlled composition and/or a normal atmosphere are also contemplated. The system may also include one or more temperature regulation devices such as a water bath, temperature controlled ovens, or other appropriate configurations and/or devices to maintain a reactor chamber at any desirable temperature range for bacterial growth.

In embodiments where a reactor chamber interior is isolated from an exterior environment, the system may include one or more seals. In the depicted embodiment, the scal corresponds to a cork, stopper, a threaded cap, a latched lid, or any other appropriate structure that seals an outlet from an interior of the reactor chamber. In this particular embodiment, a power source is electrically connected to the anode and cathode via two or more electrical leads that pass through one or more pass throughs in the seal to apply a potential to and pass a current IDC to split water within the solution into hydrogen and oxygen through an oxygen evolution reaction (OER) at the anode and a hydrogen evolution reaction (HER) at the cathode. While the leads can pass through the seal, it should be understood that embodiments in which the leads pass through a different portion of the system, such as a wall of the reactor chamber, are also contemplated as the disclosure is not so limited.

In some embodiments of any of the aspects, the at least one reactor chamber (e.g., a single reactor chamber, or primary and/or secondary reactor chamber) further comprises a power source comprising a renewable source of energy. In some embodiments of any of the aspects, the single reactor chamber further comprises a power source comprising a renewable source of energy. In some embodiments of any of the aspects, the primary reactor chamber further comprises a power source comprising a renewable source of energy. In some embodiments of any of the aspects, the secondary reactor chamber further comprises a power source comprising a renewable source of energy. In some embodiments of any of the aspects, the primary and secondary reactor chamber further comprises a power source comprising a renewable source of energy.

Depending on the particular embodiment, the above-described power source may correspond to any appropriate source of electrical current that is applied to the electrodes. However, in at least one embodiment, the power source may correspond to a renewable source of energy such as a solar cell, wind turbine, or any other appropriate source of current though embodiments in which a non-renewable energy source, such as a generator, battery, grid power, or other power source is used are also contemplated. In either case, a current from the power source is passed through the electrodes and solution to evolve hydrogen and oxygen. The current may be controlled to produce hydrogen and/or oxygen at a desired rate of production as noted above.

Accordingly, in one aspect, described herein is a system comprising: (a) at least one reactor chamber (e.g., a single reactor chamber or a primary reactor chamber) with a solution contained therein, wherein the solution comprises hydrogen (H₂) and carbon dioxide (CO₂); (b) a bacterium as described herein: (c) a pair of electrodes in contact with the solution that split water to form the hydrogen; and (d) comprising a power source comprising a renewable source of energy.

In one aspect, described herein is a system comprising: (a) at least one reactor chamber (e.g., a single reactor chamber or a primary reactor chamber) with a solution contained therein, wherein the solution comprises hydrogen (H₂) carbon dioxide (CO₂), and oxygen (O₂); (b) a bacterium as described herein: (c) a pair of electrodes in contact with the solution that split water to form the hydrogen; and (d) comprising a power source comprising a renewable source of energy.

In one aspect, described herein is a system comprising: (a) at least one reactor chamber (e.g., a single reactor chamber or a secondary reactor chamber) with a solution contained therein, wherein the solution comprises hydrogen (H₂) and carbon dioxide (CO₂); (b) a bacterium as described herein: (c) a pair of electrodes in contact with the solution that split water to form the hydrogen; and (d) comprising a power source comprising a renewable source of energy.

In one aspect, described herein is a system comprising: (a) at least one reactor chamber (e.g., a single reactor chamber or a secondary reactor chamber) with a solution contained therein, wherein the solution comprises carbon dioxide (CO₂), hydrogen (H₂), and oxygen (O₂); (b) a bacterium as described herein: (c) a pair of electrodes in contact with the solution that split water to form the hydrogen; and (d) comprising a power source comprising a renewable source of energy.

In one aspect, described herein is a system comprising: (a) at least one reactor chamber (e.g., a single reactor chamber or a secondary reactor chamber) with a solution contained therein, wherein the solution comprises an organic carbon source, hydrogen (H₂), and oxygen (O₂) and optionally carbon dioxide (CO₂); (b) a bacterium as described herein: (c) a pair of electrodes in contact with the solution that split water to form the hydrogen; and (d) comprising a power source comprising a renewable source of energy.

In one aspect, described herein is a system comprising: (a) at least one reactor chamber (e.g., a single reactor chamber or a secondary reactor chamber) with a solution contained therein, wherein the solution comprises an organic carbon source and oxygen (O₂); (b) a bacterium as described herein: (c) a pair of electrodes in contact with the solution that split water to form the hydrogen; and (d) comprising a power source comprising a renewable source of energy.

In some embodiments, the electrodes may be coated with, or formed from, a water splitting catalyst to further facilitate water splitting and/or reduce the voltage applied to the solution. In some embodiments, the catalysts may be coated onto an electrode substrate including, for example, carbon fabrics, porous carbon foams, porous metal foams, metal fabrics, solid electrodes, and/or any other appropriate geometry or material as the disclosure is not so limited. In another embodiment, the electrodes may simply be made from a desired catalyst material. Several appropriate materials for use as catalysts include, but are not limited to, one or more of a cobalt-phosphorus (Co—P) alloy, cobalt phosphate (CoPi), cobalt oxide, cobalt hydroxide, cobalt oxyhydroxide, a NiMoZn alloy, or any other appropriate material. As noted further below, certain catalysts offer additional benefits as well. For example, in one specific embodiment, the electrodes may correspond to a cathode including a cobalt-phosphorus alloy and an anode including cobalt phosphate, which may help to reduce the presence of reactive oxygen species and/or metal ions within a solution. A composition of the CoPi coating and/or electrode may include phosphorous compositions between or equal to 0 weight percent (wt %) and 50 wt %. Additionally, the Co—P alloy may include between 80 wt % and 99 wt % Co as well as 1 wt % and 20 wt % P. However, embodiments in which different element concentrations are used and/or other types of catalysts and/or electrodes are used are also contemplated as the disclosure is not so limited. For example, stainless steel, platinum, and/or other types of electrodes may be used.

In some embodiments, it may be desirable to either continuously, or periodically, bubble. i.e. sparge or flush, one or more gases through a solution and/or to refresh a composition of gases located within a headspace of the reactor chamber above a surface of the solution. In such an embodiment, a gas source may be in fluid communication with one or more gas inlets that pass through either a seal and/or another portion of the reactor chamber such as a side wall to place the gas source in fluid communication with an interior of the reactor chamber. Additionally, in some embodiments, one or more inlets discharge a flow of gas into the solution so that the gas will bubble through the solution. However, embodiments in which the one or more gas inlets discharge a flow of gas into the headspace of the reactor chamber instead are also contemplated as the disclosure is not so limited. Additionally, one or more corresponding gas outlets may be formed in a seal and/or another portion of the reactor chamber to permit a flow of gas to flow from an interior to an exterior of the reactor chamber. It should be noted that gas inlets and outlets may correspond to any appropriate structure including, but not limited to, tubes, pipes, flow passages, ports in direct fluid communication with the reactor chamber interior, or any other appropriate structure.

In some embodiments of any of the aspects, the at least one reactor chamber (e.g., a single reactor chamber, or primary and/or secondary reactor chamber) further comprises an isolated gas volume above a surface of the first and/or second solution within a headspace of the at least one reactor chamber (e.g., a single reactor chamber, or primary and/or secondary reactor chamber). In some embodiments of any of the aspects, the at least one reactor chamber (e.g., a single reactor chamber or primary reactor chamber) further comprises an isolated gas volume above a surface of the at least one growth solution (e.g., the first solution) within a headspace of the at least one reactor chamber (e.g., a single reactor chamber or primary reactor chamber). In some embodiments of any of the aspects, the at least one reactor chamber (e.g., the single reactor chamber or secondary reactor) chamber further comprises an isolated gas volume above a surface of the at least one production solution (e.g., the second solution) within a headspace of the at least one reactor chamber (e.g., the single reactor chamber or secondary reactor chamber).

In some embodiments of any of the aspects, the isolated gas volume comprises carbon dioxide (CO₂), hydrogen (H₂), and/or oxygen (O₂). In some embodiments of any of the aspects, the isolated gas volume comprises carbon dioxide (CO₂). In some embodiments of any of the aspects, the isolated gas volume comprises hydrogen (H₂). In some embodiments of any of the aspects, the isolated gas volume comprises oxygen (O₂). In some embodiments of any of the aspects, the isolated gas volume comprises carbon dioxide (CO₂) and hydrogen (H₂). In some embodiments of any of the aspects, the isolated gas volume comprises carbon dioxide (CO₂) and oxygen (O₂). In some embodiments of any of the aspects, the isolated gas volume comprises hydrogen (H₂) and oxygen (O₂). In some embodiments of any of the aspects, the isolated gas volume comprises carbon dioxide (CO₂), hydrogen (H₂), and oxygen (O₂).

Gas sources may correspond to any appropriate gas source capable of providing a pressurized flow of gas to the chamber through the inlet including, for example, one or more pressurized gas cylinders. While a gas source may include any appropriate composition of one or more gasses, in one embodiment, a gas source may provide one or more of hydrogen, nitrogen, carbon dioxide, and oxygen. The flow of gas provided by the gas source may have a composition equivalent to the range of gas compositions described above for the gas composition with a headspace of the reactor chamber. Further, in some embodiments, the gas source may simply be a source of carbon dioxide. Of course embodiments in which a different mix of gases, other including different gases and/or different concentrations than those noted above, is bubbled through a solution or otherwise input into a reactor chamber are also contemplated as the disclosure is not so limited. Additionally, the gas source may be used to help maintain operation of a reactor at, below, and/or above atmospheric pressure as the disclosure is not limited to any particular pressure range.

The above noted one or more gas inlets and outlets may also include one or more valves located along a flow path between the gas source and an exterior end of the one or more outlets. These valves may include for example, manually operated valves, pneumatically or hydraulically actuated valves, unidirectional valves (i.e. check valves) may also be incorporated in the one or more inlets and/or outlets to selectively prevent the flow of gases into or out of the reactor either entirely or in the upstream direction into the chamber and/or towards the gas source.

While the use of inlet and/or outlet gas passages have been described above, embodiments in which there are no inlet and/or outlets for gasses are present are also contemplated. For example, in one embodiment, a system including a scalable reactor may simply be flushed with appropriate gasses prior to being sealed. The system may then be flushed with an appropriate composition of gasses at periodic intervals to refresh the desired gas composition in the solution and/or headspace prior to rescaling the reactor chamber. Alternatively, the headspace may be sized to contain a gas volume sufficient for use during an entire production run.

In instances where electrodes are run at high enough rates and/or for sufficient durations, concentration may be formed within a solution in a reactor chamber. Accordingly, it may be desirable to either prevent and/or mitigate the presence of concentration gradients in the solution. Therefore, in some embodiments, a system may include a mixer such as a stir bar. Alternatively, a shaker table, and/or any other way of inducing motion in the solution to reduce the presence of concentration gradients may also be used as the disclosure is not so limited.

Embodiments in which a flow-through reaction chamber with two or more corresponding electrodes immersed in a solution that is flowed through the reaction chamber and past the electrodes are contemplated. For example, one possible embodiment, one or more corresponding electrodes may be suspended within a solution flowing through a chamber, tube, passage, or other structure. Similar to the above embodiment, the electrodes are electrically coupled with a corresponding power source to perform water splitting as the solution flows past the electrodes. Such a system may either be a single pass flow through system and/or the solution may be continuously flowed passed the electrodes in a continuous loop though other configurations are also contemplated as well.

Without wishing to be bound by theory, described herein is one possible pathway for a system to produce one or more desired products. In the depicted embodiment, the hydrogen evolution reaction occurs at the cathode. During the reaction at the cathode, two hydrogen ions (HT) are combined with two electrons to form hydrogen gas H₂ that dissolves within the solution along with carbon dioxide (CO₂), which dissolved in the solution as well. At the same time various toxicants such as reactive oxygen species (ROS) including, for example, hydrogen peroxide (H₂O₂), superoxides (O₂⁻), and/or hydroxyl radical (HO.) species as well as metallic ions may be generated at the cathode. For example. Co²⁺ ions may be dissolved into solution when a cobalt based cathode is used. As described further below, in some embodiments, the use of certain catalysts may help to reduce the production of ROS and the metallic ions leached into the solution may be deposited onto the anode using one or more elements located within the solution to form compounds such as a cobalt phosphate.

Once hydrogen and carbon dioxide are provided within a solution, bacteria present within the solution may be used to transform these compounds into useful products (e.g., triacylglycerides). For example, in one embodiment, the bacteria use hydrogenase to metabolize the dissolved hydrogen gas and one or more appropriate enzymes, such as RuBisCO or other appropriate enzyme, to provide a carbon fixation pathway. This may include absorbing the carbon dioxide and forming Acetyl-CoA through the Calvin cycle. Further, depending on the concentration of nitrogen within the solution, the bacteria may either form biomass or one or more desired products. For instance, if a concentration of nitrogen within the solution is below a predetermined nitrogen concentration threshold, the bacteria may form one or more products (e.g., triacylglycerides).

Depending on the embodiment, a solution placed in the chamber of a reactor may include water with one or more additional solvents, compounds, and/or additives. For example, the solution may include: inorganic salts such as phosphates including sodium phosphates and potassium phosphates; trace metal supplements such as iron, nickel, manganese, zinc, copper, and molybdenum; or any other appropriate component in addition to the dissolved gasses noted above. In one such embodiment, a phosphate may have a concentration between 9 and 90 mM. 9 and 72 mM. 9 and 50 mM, or any other appropriate concentration. In a particular embodiment, a water based solution may include one or more of the following in the listed concentrations: 12 mM to 123 mM of Na₂HPO₄, 11 mM to 33 mM of KH₂PO₄, 1.25 mM to 15 mM of (NH₄)₂SO₄, 0.16 mM to 0.64 mM of MgSO₄, 2.4 μM to 5.8 M of CaSO₄, 1 μM to 4 μM of NiSO₄, 0.81 μM to 3.25 μM molar concentration of Ferric Citrate. 60 mM to 240 mM molar concentration of NaHCO₃.

Reactive oxygen species (ROS) as well as metallic ions may be formed and/or dissolved into a solution during the hydrogen evolution reaction at the cathode. However. ROS and larger concentrations of the metallic ions within the solution may be detrimental to cell growth above certain concentrations. It is noted that the use of continuous hydrogen production within a reactor to form hydrogen for conversion into one or more desired products has been hampered by the production of these ROS and metallic ion concentrations because the bacteria used to form the desired products tend to be sensitive to these compounds and ions limiting the growth of, and above certain concentrations, killing the bacteria. Therefore, in some embodiments, it may be desirable to apply voltages, use electrodes that produce less ROS, remove and/or prevent the dissolution of metallic ions from the electrodes, and/or use bacteria that are resistant to the presence of these toxicants as detailed further below.

As noted above, it may be desirable to select one or more catalysts for use as the electrodes that produce fewer reactive oxygen species (ROS) during use. Specifically, a biocompatible catalyst system that is not toxic to the bacterium and lowers the overpotential for water splitting may be used in some embodiments. One such example of a catalyst includes a ROS-resistant cobalt-phosphorus (Co—P) alloy cathode. This cathode may be combined with a cobalt phosphate (CoPi) anode. This catalyst pair has the added benefit of the anode being self-healing. In other words, the catalyst pair helps to remove metallic Co²⁺ ions present with a solution in a reactor. Without wishing to be bound by theory, the electrode pair works in concert to remove extracted metal ions from the cathode by depositing them onto the anode which may help to maintain extraneous cobalt ions at relatively low concentrations within solution and to deliver a low applied electrical potential to split water to generate H₂. Without wishing to be bound by theory, it is believed that during electrolysis of the water, phosphorus and/or cobalt is extracted from the electrodes. The reduction potential of leached cobalt is such that formation of cobalt phosphate using phosphate available in the solution is energetically favored. Cobalt phosphate formed in solution then deposits onto the anode at a rate linearly proportional to free Co²⁺, providing a self-healing process for the electrodes. In view of the above, the cobalt-phosphorus (Co—P) alloy and cobalt phosphate (CoPi) catalysts may be used to help mitigate the presence of both ROS and metal ions within the solution to help promote growth of bacteria within the reactor chamber.

It should be understood that any appropriate voltage may be applied to a pair of electrodes immersed in a solution to split water into hydrogen and oxygen. However, in some embodiments, the applied voltage may be limited to fall between upper and lower voltage thresholds. For example, the self-healing properties of a cobalt phosphate and cobalt phosphorous based alloy electrode pair may function at voltage potentials greater than about 1.42 V. Additionally, the thermodynamic minimum potential for splitting water is about 1.23 V. Therefore, depending on the particular embodiment, the voltage applied to the electrodes may be greater than or equal to about 1.23 V. 1.42 V. 1.5 V. 2 V. 2.2 V. 2.4 V, or any other appropriate voltage. Additionally, the applied voltage may be less than or equal to about 10 V. 5 V. 4 V. 3 V. 2.9 V. 2.8 V. 2.7 V. 2.6 V. 2.5 V, or any other appropriate voltage. Combinations of the above noted voltage ranges are contemplated including, for example, a voltage applied to a pair of electrodes may be between 1.23 V and 10 V. 1.42 V and 5 V. 2 V and 3 V. 2.3 V and 2.7 V as well as other appropriate ranges. Additionally, it should be understood that voltages both greater than and less than those noted above, as well as different combinations of the above ranges, are also contemplated as the disclosure is not so limited. In addition to the applied voltages, any appropriate current may be passed through the electrodes to perform water splitting which will depend on the desired rate of hydrogen generation for a given volume of a reactor being used. For example, in some embodiments, a current used to split water may be controlled to generate hydrogen at a rate substantially equal to a rate of hydrogen consumption by bacteria in the solution. However, embodiments in which hydrogen is produced at rates both greater than or less than consumption by the bacteria are also contemplated.

In addition to using catalysts, controlling the solution pH, and applying appropriate driving potentials, and/or controlling any other appropriate parameter to reduce the presence of reactive oxygen species (ROS) within the solution in a reaction chamber, it may also be desirable to use bacteria that are resistant to the presence of ROS and/or metallic ions present within the solution as noted previously. Specifically, a chemolithoautotrophic bacterium that is resistant to reactive oxygen species may be used. Further, in some embodiments a R. eutropha bacteria that is resistant to ROS as compared to a wild-type H₁₆ R. eutropha may be used. US 2018/0265898 details several genetic polymorphisms found between the wild-type H₁₆ R. eutropha and a ROS-tolerant BC4 strain that was purposefully evolved. Mutations of the BC4 strain relative to the wild type bacteria are detailed further below (see e.g., Table 2 and SEQ ID NOs: 3-6).

In some embodiments of any of the aspects, the systems described herein are capable of undergoing intermittent production. For example, when a driving potential is applied to the electrodes to generate hydrogen, the bacteria produce the desired product. Correspondingly, when the potential is removed and hydrogen is no longer generated, production of the product is ceased once the available hydrogen is consumed and a reduction in overall biomass is observed until the potential is once again applied to the electrodes to generate hydrogen. The system will then resume biomass and/or product formation. Thus, while a system may be run continuously to produce a desired product, in some modes of operation a driving potential may be intermittently applied to the electrodes to intermittently split water to form hydrogen and correspondingly intermittently produce a desired product. A frequency of the intermittently applied potential may be any frequency and may either be uniform or non-uniform as the disclosure is not so limited. This ability to intermittently produce a product may be desirable in applications such as when intermittent renewable energy sources are used to provide the power applied to the electrodes including, but not limited to, intermittent power sources such as solar and wind energy. In some embodiments of any of the aspects, the primary reactor chamber is used for continuous bacterial biomass production. In some embodiments of any of the aspects, the secondary reactor chamber is used for fed batch bioproduct production.

In some embodiments of any of the aspects, the systems described herein can be scaled up to meet bioproduction needs. As used herein, the term “scale up” refers to an increase in production capacity (e.g., of a system as described herein). In some embodiments of the aspects, a system (e.g., a bioreactor system) as described herein can be scaled up by at least 1-fold, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 10⁰-fold, at least 1,000-fold, at least 10^{,000}-fold, at least 100,000-fold, or at least 1,000,000-fold.

In some embodiments of the aspects, a bioreactor system as described herein can be scaled up to at least a 100 ml reactor, at least a 500 ml reactor, at least a 1000 mL reactor, at least a 2 L reactor, at least a 5 L reactor, at least a 10 L reactor, at least a 25 L reactor, at least a 50 L reactor, at least a 100 L reactor, at least a 500 L reactor, or at least a 1,000 L reactor. In some embodiments of the aspects, the primary reactor chamber is at least a 100 ml reactor, at least a 500 ml reactor, at least a 1000 mL reactor, at least a 2 L reactor, at least a 5 L reactor, at least a 10 L reactor, at least a 25 L reactor, at least a 50 L reactor, at least a 100 L reactor, at least a 500 L reactor, or at least a 1,000 L reactor. In some embodiments of the aspects, the secondary reactor chamber is at least a 100 ml reactor, at least a 500 ml reactor, at least a 1000 mL reactor, at least a 2 L reactor, at least a 5 L reactor, at least a 10 L reactor, at least a 25 L reactor, at least a 50 L reactor, at least a 100 L reactor, at least a 500 L reactor, at least a 1,000 L reactor, at least a 10,000 L reactor, at least a 100,000 L reactor, or at least a 1,000,000 L reactor.

Described herein are methods of culturing bacteria and/or sustainably producing bioproducts. In one aspect, the method comprises: (a) culturing the bacterium in at least one reactor chamber with at least one growth solution contained therein, wherein the at least one growth solution comprises: (i) carbon dioxide (CO₂), hydrogen (H₂), and oxygen (O₂); or (ii) an organic carbon source, hydrogen (H₂), and oxygen (O₂), and optionally carbon dioxide (CO₂); (b) adding at least one production solution to the at least one reactor chamber, wherein the at least one production solution comprises: (i) carbon dioxide (CO₂), hydrogen (H₂), and oxygen (O₂); (ii) an organic carbon source, hydrogen (H₂), and oxygen (O₂), and optionally carbon dioxide (CO₂); or (iii) an organic carbon source and oxygen (O₂); and (c) culturing the bacterium in the at least one production solution.

In some embodiments of any of the aspects, the method comprises using at least one growth solution and at least one production solution (see e.g., Table 5). In some embodiments of any of the aspects, the system comprises one growth solution and one production solution. In some embodiments of any of the aspects, the method comprises using one growth solution and two production solutions. In some embodiments of any of the aspects, the method comprises using two growth solutions and one production solution. In some embodiments of any of the aspects, the method comprises using two growth solutions and two production solutions. In some embodiments of any of the aspects, the first growth solution can be used for a pre-determined period of time, and then a second growth solution can be used. In some embodiments of any of the aspects, the first production solution can be used for a pre-determined period of time, and then a second production solution can be used. See e.g., Table 4 and Table 5 for exemplary combinations of conditions/solutions for the methods described herein.

In one aspect, the method comprises: (a) culturing the bacterium in at least one reactor chamber with at least one growth solution contained therein, wherein the at least one growth solution comprises: (i) carbon dioxide (CO₂), hydrogen (H₂), and oxygen (O₂); or (ii) an organic carbon source, hydrogen (H₂), and oxygen (O₂), and optionally carbon dioxide (CO₂); (b) adding at least one production solution to the at least one reactor chamber, wherein the at least one production solution comprises: (i) carbon dioxide (CO₂), hydrogen (H₂), and oxygen (O₂); (ii) an organic carbon source, hydrogen (H₂), and oxygen (O₂), and optionally carbon dioxide (CO₂); or (iii) an organic carbon source and oxygen (O₂); (c) culturing the bacterium in the at least one production solution; and (d) isolating, collecting, or concentrating the bioproduct from the bacterium in the at least one reactor chamber or from the at least one production solution in the at least one reactor chamber.

In one aspect, the method comprises: (a) culturing a bacterium (e.g., a bacterium engineered to produce the bioproduct) in a culture medium comprising CO₂ and/or H₂; and (b) isolating, collecting, or concentrating bioproducts from said bacterium or from the culture medium of said bacterium. In another aspect, described herein are methods of sustainably producing the bioproduct comprising: (a) culturing a bacterium (e.g., a bacterium engineered to produce the bioproduct) in a culture medium comprising a simple organic carbon source (e.g., glycerol) and/or H₂; and (b) isolating, collecting, or concentrating the bioproduct from said bacterium or from the culture medium of said bacterium. In some embodiments of any of the aspects, the culture medium comprises CO₂ and glycerol.

In one aspect, described herein is a method of a culturing a bacterium, comprising: (a) culturing the bacterium in a primary reactor chamber with at least one growth solution (e.g., a first solution) contained therein, wherein the at least one growth solution (e.g., the first solution) comprises: (i) carbon dioxide (CO₂), hydrogen (H₂), and oxygen (O₂), or (ii) an organic carbon source, hydrogen (H₂), and oxygen (O₂), and optionally carbon dioxide (CO₂); (b) moving at least a portion of the at least one growth solution (e.g., the first solution) from the primary reactor chamber into at least one secondary reactor chamber with at least one production solution (e.g., a second solution) contained therein, wherein the at least one production solution (e.g., the second solution) comprises: (i) carbon dioxide (CO₂), hydrogen (H₂), and oxygen (O₂); (ii) an organic carbon source, hydrogen (H₂), and oxygen (O₂) and optionally carbon dioxide (CO₂); or (iii) an organic carbon source and oxygen (O₂); and (C) culturing the bacterium in the secondary reactor chamber.

In one aspect, described herein is a method of producing a bioproduct, comprising: (a) culturing a bacterium that produces a bioproduct in a primary reactor chamber with at least one growth solution (e.g., a first solution) contained therein, wherein the at least one growth solution (e.g., the first solution) comprises: (i) carbon dioxide (CO₂), hydrogen (H₂), and oxygen (O₂), or (ii) an organic carbon source, hydrogen (H₂), and oxygen (O₂), and optionally carbon dioxide (CO₂); (b) moving at least a portion of the at least one growth solution (e.g., the first solution) from the primary reactor chamber into a secondary reactor chamber with at least one production solution (e.g., a second solution) contained therein, wherein the at least one production solution (e.g., the second solution) comprises: (i) carbon dioxide (CO₂), hydrogen (H₂), and oxygen (O₂); (ii) an organic carbon source, hydrogen (H₂), and oxygen (O₂) and optionally carbon dioxide (CO₂); or (iii) an organic carbon source and oxygen (O₂); (c) culturing the bacterium in the secondary reactor chamber; and (d) isolating, collecting, or concentrating the bioproduct from the bacterium in the secondary reactor chamber or from the at least one production solution (e.g., the second solution) in the second reactor chamber.

In multiple aspects, described herein are methods of adapting the metabolism of a bacterium for gas fermentation. Organic carbon fermentation permits expression of pathways that allow for inorganic carbon uptake and energy generation. In one aspect, described herein is a method of adapting the metabolism of a bacterium for gas fermentation, the method comprising: (a) culturing the bacterium in a solution comprising an organic carbon source; and (b) transitioning the bacterium to a gas fermentation solution lacking an organic carbon source once the bacterium grows to a pre-determined concentration.

In one aspect, described herein is a method of adapting the metabolism of a bacterium for gas fermentation, the method comprising: (a) culturing the bacterium in a growth solution comprising an organic carbon source; and (b) transitioning the bacterium to a production solution lacking an organic carbon source once the bacterium grows to a pre-determined concentration.

In one aspect, described herein is a method of adapting the metabolism of a bacterium for gas fermentation, the method comprising: (a) culturing the bacterium in at least one growth solution comprising an organic carbon source, wherein the at least one growth solution comprises: an organic carbon source, hydrogen (H₂), and oxygen (O₂), and optionally carbon dioxide (CO₂); and (b) transitioning the bacterium to at least one production solution lacking an organic carbon source once the bacterium grows to a pre-determined concentration, wherein the at least one production comprises: carbon dioxide (CO₂), hydrogen (H₂), and oxygen (O₂).

In one aspect, described herein is a method of adapting the metabolism of a bacterium for gas fermentation, the method comprising: (a) culturing the bacterium in at least one growth solution comprising an organic carbon source, wherein the at least one growth solution comprises: (i) an organic carbon source, hydrogen (H₂), and oxygen (O₂), and optionally carbon dioxide (CO₂), or (ii) an organic carbon source and oxygen (O₂); and (b) transitioning the bacterium to at least one production solution lacking an organic carbon source once the bacterium grows to a pre-determined concentration, wherein the at least one production comprises: carbon dioxide (CO₂), hydrogen (H₂), and oxygen (O₂).

In one aspect, described herein is a method of adapting the metabolism of a bacterium for gas fermentation, the method comprising: (a) culturing the bacterium in at least one mixotrophic growth solution comprising an organic carbon source; and (b) transitioning the bacterium to at least one gas fermentation production solution lacking an organic carbon source once the bacterium grows to a pre-determined concentration.

In some embodiments of any of the aspects, the solution in the at least one reactor chamber (e.g., a single reactor chamber or primary reactor chamber) comprises: carbon dioxide (CO₂), hydrogen (H₂), and oxygen (O₂). In some embodiments of any of the aspects, the solution in the at least one reactor chamber (e.g., a single reactor chamber or primary reactor chamber) comprises: an organic carbon source, hydrogen (H₂), and oxygen (O₂), and optionally carbon dioxide (CO₂). In some embodiments of any of the aspects, the gasses in the solution in the at least one reactor chamber (e.g., a single reactor chamber or primary reactor chamber) comprises: carbon dioxide (CO₂), hydrogen (H₂), and oxygen (O₂). In some embodiments of any of the aspects, the gasses in the solution in the at least one reactor chamber (e.g., a single reactor chamber or primary reactor chamber) comprises: hydrogen (H₂), and oxygen (O₂), and optionally carbon dioxide (CO₂). In some embodiments of any of the aspects, the gasses in the solution in the at least one reactor chamber (e.g., a single reactor chamber or primary reactor chamber) comprises: hydrogen (H₂) and oxygen (O₂). In some embodiments of any of the aspects, the gasses in the solution in the at least one reactor chamber (e.g., a single reactor chamber or primary reactor chamber) comprises: hydrogen (H₂), and oxygen (O₂), and carbon dioxide (CO₂).

In some embodiments of any of the aspects, the solution in the at least one reactor chamber (e.g., the single reactor chamber or secondary reactor chamber) comprises: carbon dioxide (CO₂), hydrogen (H₂), and oxygen (O₂). In some embodiments of any of the aspects, the gasses in the solution in the at least one reactor chamber (e.g., the single reactor chamber or secondary reactor chamber) comprises: carbon dioxide (CO₂), hydrogen (H₂), and oxygen (O₂). In some embodiments of any of the aspects, the solution in the at least one reactor chamber (e.g., the single reactor chamber or secondary reactor chamber) comprises: an organic carbon source, hydrogen (H₂), and oxygen (O₂) and optionally carbon dioxide (CO₂). In some embodiments of any of the aspects, the gasses in the solution in the at least one reactor chamber (e.g., the single reactor chamber or secondary reactor chamber) comprises: hydrogen (H₂), and oxygen (O₂) and optionally carbon dioxide (CO₂). In some embodiments of any of the aspects, the gasses in the solution in the at least one reactor chamber (e.g., the single reactor chamber or secondary reactor chamber) comprises: hydrogen (H₂) and oxygen (O₂). In some embodiments of any of the aspects, the gasses in the solution in the at least one reactor chamber (e.g., the single reactor chamber or secondary reactor chamber) comprises: hydrogen (H₂), and oxygen (O₂) and carbon dioxide (CO₂). In some embodiments of any of the aspects, the solution in the at least one reactor chamber (e.g., the single reactor chamber or secondary reactor chamber) comprises: an organic carbon source and oxygen (O₂). In some embodiments of any of the aspects, the gas in the solution in the at least one reactor chamber (e.g., the single reactor chamber or secondary reactor chamber) comprises oxygen (O₂).

In some embodiments of any of the aspects, the bacterium is cultured in the at least one reactor chamber (e.g., a single reactor chamber or primary reactor chamber) for a sufficient amount of time and under sufficient conditions for the bacterium to grow to a pre-determined concentration. As a non-limiting example, the sufficient amount of time for the bacterium to grow to a pre-determined concentration in the at least one reactor chamber (e.g., a single reactor chamber or primary reactor chamber) is at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 24 hours, at least 2.5 days, at least 3 days, at least 3.5 days, at least 4 days, at least 4.5 days, at least 5 days, at least 5.5 days, at least 6 days, at least 6.5 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, 0-1 weeks, 1-2 weeks, 2-3 weeks, 3-4 weeks, or 0.05-30 days. In some embodiments of any of the aspects, the sufficient amount of time for the bacterium to grow to a pre-determined concentration in the at least one reactor chamber (e.g., a single reactor chamber or primary reactor chamber) is about 23 hours. In some embodiments of any of the aspects, the sufficient amount of time for the bacterium to grow to a pre-determined concentration in the at least one reactor chamber (e.g., a single reactor chamber or primary reactor chamber) is about 36 hours.

In some embodiments of any of the aspects, the bacterium is cultured sequentially in two different growth solutions for a sufficient amount of time and under sufficient conditions for the bacterium to grow to a pre-determined concentration. In some embodiments of any of the aspects, the bacterium is cultured in a first gas fermentation growth solution and then in a second mixotrophic growth solution (see e.g., Table 5). In some embodiments of any of the aspects, the bacterium is cultured in a first mixotrophic growth solution and then in a second gas fermentation growth solution (see e.g., Table 5).

In some embodiments of any of the aspects, the bacterium is cultured in the first growth solution (e.g., gas fermentation or mixotrophic growth) for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 24 hours, at least 2.5 days, at least 3 days, at least 3.5 days, at least 4 days, at least 4.5 days, at least 5 days, at least 5.5 days, at least 6 days, at least 6.5 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, 0-1 weeks, 1-2 weeks, 2-3 weeks, 3-4 weeks, or 0.05-30 days.

In some embodiments of any of the aspects, the bacterium is cultured in the second growth solution (e.g., gas fermentation or mixotrophic growth) for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 24 hours, at least 2.5 days, at least 3 days, at least 3.5 days, at least 4 days, at least 4.5 days, at least 5 days, at least 5.5 days, at least 6 days, at least 6.5 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, 0-1 weeks, 1-2 weeks, 2-3 weeks, 3-4 weeks, or 0.05-30 days.

In some embodiments of any of the aspects, the sufficient conditions for the bacterium to grow to a pre-determined concentration in the at least one reactor chamber (e.g., a single reactor chamber or primary reactor chamber) includes a minimal medium, a defined medium, or a rich medium, as described herein or known in the art. In some embodiments of any of the aspects, the pre-determined concentration of bacteria in the primary reactor chamber is at least 10¹ colony-forming units per milliliter (CFU/mL), at least 10² CFU/mL, at least 10³ CFU/mL, at least 10⁴ CFU/mL, at least 105 CFU/mL, at least 10⁶ CFU/mL, at least 10⁷ CFU/mL, at least 10⁸ CFU/mL, at least 10⁹ CFU/mL, at least 10¹⁰ CFU/mL, at least 10¹¹ CFU/mL, or at least 10¹² CFU/mL or more. In some embodiments of any of the aspects, the pre-determined concentration of bacteria in the at least one reactor chamber (e.g., a single reactor chamber or primary reactor chamber) is an OD600 measurement of at least 0.1, at least 0.2, at least 0.3, at least 0.4, at least 0.5, at least 0.6, at least 0.7, at least 0.8, at least 0.9, or at least 1.0, or more.

In some embodiments of any of the aspects, the bacterium does not produce the bioproduct in the at least one reactor chamber (e.g., a single reactor chamber or primary reactor chamber). In some embodiments of any of the aspects, the bacterium only produces the bioproduct when it is induced to do so (e.g., after introduction of an inducer; e.g., in that at least one reactor chamber; e.g., in a single reactor chamber or the at least one secondary reactor chamber).

In some embodiments of any of the aspects, after culturing, growth, and/or fermentation of or by the bacterium has occurred in the at least one reactor chamber (e.g., a single reactor chamber or primary reaction chamber), the at least one reactor chamber (e.g., a single reactor chamber or primary reaction chamber) comprises a metabolized first solution (e.g., an at least partially metabolized first solution). As compared to the at least one growth solution (e.g., the first solution), the metabolized solution further comprises a higher concentration of bacteria and may optionally include less starting material (e.g., carbon dioxide (CO₂), hydrogen (H₂), and oxygen (O₂)) and more organic carbon sources or other products produced by gas fermentation and/or chemolithotrophy (see e.g., Table 1). In some embodiments of any of the aspects, at least a portion of the metabolized production solution (e.g., metabolized first solution) from the at least one reactor chamber (e.g., primary reaction chamber) is removed or moved into at least one other reactor chamber (e.g., a secondary reactor chamber).

In some embodiments of any of the aspects, at least a portion of the at least one growth solution (e.g., an at least partially metabolized growth solution or an at least partially metabolized first solution) from the at least one reactor chamber (e.g., a single reactor chamber or primary reactor chamber) is removed or moved into the at least one other reactor chamber (e.g., at least one secondary reactor chamber) after the bacterium grows to a pre-determined concentration. In some embodiments of any of the aspects, at least 1%, at least 2%, at least 3%, at least 4%, at least 5%, at least 6%, at least 7%, at least 8%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, or more of the at least one growth solution (e.g., an at least partially metabolized growth solution or an at least partially metabolized first solution) from the at least one reactor chamber (e.g., a single reactor chamber or primary reactor chamber) is removed or moved into the at least one other reactor chamber (e.g., at least one secondary reactor chamber) after the bacterium grows to a pre-determined concentration. In some embodiments of any of the aspects, at least a portion of the at least one growth solution (e.g., an at least partially metabolized growth solution or an at least partially metabolized first solution) from the at least one reactor chamber (e.g., a single reactor chamber or primary reactor chamber) is removed or moved into the 1, 2, 3, 4, 5, 6, 7, 8, 9, 10, or more other reactor chamber(s) (e.g., secondary reactor chamber(s)) after the bacterium grows to a pre-determined concentration.

In some embodiments of any of the aspects, the process of removing or moving at least a portion of the at least one growth solution (e.g., an at least partially metabolized first solution) from the at least one reactor chamber (e.g., a single reactor chamber or primary reactor chamber) into at least one secondary reactor chamber is iterative.

In some embodiments of any of the aspects, the method comprises the following iterative (e.g., and sequential) steps: (a) a portion of the at least one growth solution (e.g., an at least partially metabolized growth solution or an at least partially metabolized first solution) from the at least one reactor chamber (e.g., a single reactor chamber or primary reactor chamber) is removed or moved into at least one other reactor chamber (e.g., a first secondary reactor chamber) after the bacterium grows to a pre-determined concentration; and (b) a portion of the at least one growth solution (e.g., an at least partially metabolized growth solution or an at least partially metabolized first solution) from the at least one reactor chamber (e.g., a single reactor chamber or primary reactor chamber) is removed or moved into at least one other reactor chamber (e.g., a second secondary reactor chamber) after the bacterium grows to a pre-determined concentration. For example, steps (a) and (b) are repeated iteratively whenever the bacterium grows to a pre-determined concentration in the at least one reactor chamber (e.g., a single reactor chamber or primary reactor chamber). As a non-limiting example, the steps are performed in the following iterative order: (a) (b) (a) (b) (a) (b), etc.

In some embodiments of any of the aspects, the method comprises the following iterative (e.g., and sequential) steps: (a) a portion of the at least one growth solution (e.g., an at least partially metabolized growth solution or an at least partially metabolized first solution) from the at least one reactor chamber (e.g., a single reactor chamber or primary reactor chamber) is removed or moved into at least one other reactor chamber (e.g., a first secondary reactor chamber) after the bacterium grows to a pre-determined concentration: (b) a portion of the at least one growth solution (e.g., an at least partially metabolized growth solution or an at least partially metabolized first solution) from the at least one reactor chamber (e.g., a single reactor chamber or primary reactor chamber) is removed or moved into at least one other reactor chamber (e.g., a second secondary reactor chamber) after the bacterium grows to a pre-determined concentration; and (c) a portion of the at least one growth solution (e.g., an at least partially metabolized growth solution or an at least partially metabolized first solution) from the at least one reactor chamber (e.g., a single reactor chamber or primary reactor chamber) is removed or moved into at least one other reactor chamber (e.g., a third secondary reactor chamber) after the bacterium grows to a pre-determined concentration. For example, steps (a), (b), and (c) are repeated iteratively whenever the bacterium grows to a pre-determined concentration in the at least one reactor chamber (e.g., a single reactor chamber or primary reactor chamber). As a non-limiting example, the steps are performed in the following iterative order: (a) (b) (c) (a) (b) (c) (a) (b) (c), etc.

In some embodiments of any of the aspects, a portion of the at least one growth solution (e.g., an at least partially metabolized growth solution or an at least partially metabolized first solution) from the at least one reactor chamber (e.g., a single reactor chamber or primary reactor chamber) is removed or moved into at least one other reactor chamber (e.g., at least one secondary reactor chamber) whenever the bacterium grows to a pre-determined concentration such that the bacterium does not ever exceed the pre-determined concentration. In some embodiments of any of the aspects, the fermentation in the at least one reactor chamber (e.g., a single reactor chamber or primary reactor chamber) is continuous as at least a portion of the at least one growth solution (e.g., an at least partially metabolized growth solution or an at least partially metabolized first solution) from the at least one reactor chamber (e.g., a single reactor chamber or primary reactor chamber) is removed or moved into the at least one other reactor chamber (e.g., at least one secondary reactor chamber) whenever the bacterium grows to a pre-determined concentration. In some embodiments of any of the aspects, after the portion of the at least one growth solution (e.g., an at least partially metabolized growth solution or an at least partially metabolized first solution) is removed from the at least one reactor chamber (e.g., a single reactor chamber or primary reactor chamber), fresh solution (e.g., cell culture medium) is added to the at least one reactor chamber (e.g., a single reactor chamber or primary reactor chamber), e.g., to dilute the remaining bacteria to a concentration below the pre-determined concentration. In some embodiments of any of the aspects, after the portion of the at least one growth solution (e.g., an at least partially metabolized growth solution or an at least partially metabolized first solution) is removed from the at least one reactor chamber (e.g., a single reactor chamber or primary reactor chamber), fresh bacterium is added to the at least one reactor chamber (e.g., a single reactor chamber or primary reactor chamber).

In some embodiments of any of the aspects, the bacterium is cultured in the at least one reactor chamber (e.g., the single reactor chamber or secondary reactor chamber) for a sufficient amount of time and under sufficient conditions for the bacterium to produce a pre-determined concentration of the bioproduct. As a non-limiting example, the sufficient amount of time for the bacterium to produce a pre-determined concentration of the bioproduct in the at least one reactor chamber (e.g., the single reactor chamber or secondary reactor chamber) is at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 24 hours, at least 2.5 days, at least 3 days, at least 3.5 days, at least 4 days, at least 4.5 days, at least 5 days, at least 5.5 days, at least 6 days, at least 6.5 days, at least 7 days, at least 8 days, at least 9 days, at least 10 days, at least 11 days, at least 12 days, at least 13 days, at least 14 days, 0-1 weeks, 1-2 weeks, or 0.05-14 days. In some embodiments of any of the aspects, the sufficient amount of time for the bacterium to produce a pre-determined concentration of the bioproduct in the at least one reactor chamber (e.g., the single reactor chamber or secondary reactor chamber) is about 26 hours. In some embodiments of any of the aspects, the sufficient amount of time for the bacterium to produce a pre-determined concentration of the bioproduct in the at least one reactor chamber (e.g., the single reactor chamber or secondary reactor chamber) is about 54 hours.

In some embodiments of any of the aspects, the bacterium is cultured sequentially in two different production solutions for a sufficient amount of time and under sufficient conditions for the bacterium to produce a pre-determined concentration of the bioproduct. In some embodiments of any of the aspects, the first production solution is a fermentation, mixotrophic, or organic carbon source first production solution (see e.g., Table 5). In some embodiments of any of the aspects, the second production solution is a fermentation, mixotrophic, or organic carbon source second production solution (see e.g., Table 5).

In some embodiments of any of the aspects, the bacterium is cultured in the first production solution (e.g., gas fermentation, mixotrophic, or organic carbon growth) for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 24 hours, at least 2.5 days, at least 3 days, at least 3.5 days, at least 4 days, at least 4.5 days, at least 5 days, at least 5.5 days, at least 6 days, at least 6.5 days, at least 7 days, at least 8 days, at least 9 days, at least 10 days, at least 11 days, at least 12 days, at least 13 days, at least 14 days, 0-1 weeks, 1-2 weeks, or 0.05-14 days.

In some embodiments of any of the aspects, the bacterium is cultured in the second production solution (e.g., gas fermentation, mixotrophic, or organic carbon growth) for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 24 hours, at least 2.5 days, at least 3 days, at least 3.5 days, at least 4 days, at least 4.5 days, at least 5 days, at least 5.5 days, at least 6 days, at least 6.5 days, at least 7 days, at least 8 days, at least 9 days, at least 10 days, at least 11 days, at least 12 days, at least 13 days, at least 14 days, 0-1 weeks, 1-2 weeks, or 0.05-14 days.

In some embodiments of any of the aspects, the sufficient conditions for the bacterium to produce a pre-determined concentration of the bioproduct in the at least one reactor chamber (e.g., the single reactor chamber or secondary reactor) chamber includes a minimal medium, a defined medium, or a rich medium, as described herein or known in the art. In some embodiments of any of the aspects, the method further comprises inducing the bacterium to produce the bioproduct. In some embodiments of any of the aspects, the method further comprises adding an inducer to the solution in the at least one reactor chamber (e.g., the single reactor chamber or secondary reactor chamber) in order to induce the bacterium to produce the bioproduct. In some embodiments of any of the aspects, the solution in the at least one reactor chamber (e.g., the single reactor chamber or at least one secondary reactor chamber) comprises an inducer.

In some embodiments of any of the aspects, the method further comprises adding at least one (e.g., 1, 2, 3,4 5, 6, 7, 8, 9, 10, or more) inducer solution(s) to the at least one reactor chamber. In some embodiments of any of the aspects, the method comprises adding one inducer solution. In some embodiments of any of the aspects, the inducer solution is used to induce the bacterium to produce at least one bioproduct in the at least one reactor chamber (e.g., a single reactor chamber or a secondary reactor chamber).

In some embodiments of any of the aspects, the at least one inducer solution is added after culturing the bacterium in at least one reactor chamber with at least one growth solution contained therein. In some embodiments of any of the aspects, the at least one inducer solution is added after the bacterium is cultured in the at least one growth solution for a sufficient amount of time and under sufficient conditions for the bacterium to grow to a pre-determined concentration. In some embodiments of any of the aspects, the at least one inducer solution is before culturing the bacterium in the at least one production solution. In some embodiments of any of the aspects, the at least one inducer solution is during the step of culturing the bacterium in the at least one production solution.

In some embodiments of any of the aspects, the inducer induces expression of the bioproduct from an inducible promoter. Non-limiting examples of inducible promoters include: a doxycycline-inducible promoter, the lac promoter, the lacUV5 promoter, the tac promoter, the trc promoter, the T5 promoter, the T7 promoter, the T7-lac promoter, the araBAD promoter, the rha promoter, the tet promoter, an isopropyl β-D-1-thiogalactopyranoside (IPTG)-dependent promoter, an AlcA promoter, a LexA promoter, a temperature inducible promoter (e.g., Hsp70 or Hsp90-derived promoters), or a light inducible promoter (e.g., pDawn/YFI/FixK2 promoter/CI/pR promoter system). In some embodiments of any of the aspects, the inducer is arabinose and the bioproduct is encoded in an arabinose-inducible vector or under the control of an arabinose-inducible promoter (e.g., pBAD).

In some embodiments of any of the aspects, a concentration of the bioavailable nitrogen in the inducer solution is below a threshold nitrogen concentration to induce or cause the bacteria to produce a product. In some embodiments of any of the aspects, an external inducer can be used to induce production of the product. Non-limiting examples of external inducers include: isopropyl β-D-1-thiogalactopyranoside (IPTG), glucose, arabinose, anhydrotetracycline, rhamnose, or xylose. In some embodiments of any of the aspects, the inducer solution is also referred to as a culture medium and can comprise a minimal medium or a defined medium as described further herein.

In some embodiments of any of the aspects, the inducer solution further comprises: carbon dioxide (CO₂), hydrogen (H₂), and oxygen (O₂). In some embodiments of any of the aspects, the inducer solution further comprises: an organic carbon source, hydrogen (H₂), and oxygen (O₂), and optionally carbon dioxide (CO₂). In some embodiments of any of the aspects, the inducer solution further comprises: an organic carbon source and oxygen (O₂).

In some embodiments of any of the aspects, the bacterium is exposed to the at least one inducer solution for at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 20 minutes, at least 30 minutes, at least 40 minutes, at least 50 minutes, at least 60 minutes, at least 70 minutes, at least 80 minutes, at least 90 minutes, at least 10⁰ minutes, at least 110 minutes, at least 120 minutes, at least 3 hours, at least 4 hours, at least 5 hours, at least 6 hours, at least 7 hours, at least 8 hours, at least 9 hours, at least 10 hours, at least 11 hours, at least 12 hours, at least 24 hours, at least 2.5 days, at least 3 days, at least 3.5 days, at least 4 days, at least 4.5 days, at least 5 days, at least 5.5 days, at least 6 days, at least 6.5 days, at least 7 days, at least 8 days, at least 9 days, at least 10 days, at least 11 days, at least 12 days, at least 13 days, at least 14 days, 0-1 weeks. 1-2 weeks, or 0.05-14 days.

In some embodiments of any of the aspects, the pre-determined concentration of bioproduct in the at least one reactor chamber (e.g., the single reactor chamber or secondary reactor chamber) or the production solution in the at least one reactor chamber (e.g., the single reactor chamber or secondary reactor chamber) is at least 1 ng/ml, at least 2 ng/mL, at least 3 ng/ml, at least 4 ng/ml, at least 5 ng/ml, at least 6 ng/ml, at least 7 ng/ml, at least 8 ng/mL, at least 9 ng/ml, at least 10 ng/ml, at least 10 ng/ml, at least 20 ng/ml, at least 30 ng/ml, at least 40 ng/ml, at least 50 ng/ml, at least 60 ng/ml, at least 70 ng/ml, at least 80 ng/ml, at least 90 ng/ml, at least 10⁰ ng/ml, at least 10⁰ ng/mL, at least 200 ng/ml, at least 300 ng/ml, at least 400 ng/ml, at least 500 ng/ml, at least 600 ng/ml, at least 700 ng/ml, at least 800 ng/mL, at least 900 ng/ml, or more.

In some embodiments of any of the aspects, the pre-determined concentration of bioproduct in the at least one reactor chamber (e.g., the single reactor chamber or secondary reactor chamber) or production solution in the at least one reactor chamber (e.g., the single reactor chamber or secondary reactor chamber) is at least 1 μg/mL, at least 2 μg/mL, at least 3 μg/mL, at least 4 μg/mL, at least 5 μg/mL, at least 6 μg/mL, at least 7 μg/mL, at least 8 μg/mL, at least 9 μg/mL, at least 10 μg/mL, at least 10 μg/mL, at least 20 μg/mL, at least 30 μg/mL, at least 40 g/mL, at least 50 μg/mL, at least 60 μg/mL, at least 70 μg/mL, at least 80 μg/mL, at least 90 μg/mL, at least 10⁰ μg/mL, at least 10⁰ μg/mL, at least 200 μg/mL, at least 300 μg/mL, at least 400 μg/mL, at least 500 μg/mL, at least 600 μg/mL, at least 700 μg/mL, at least 800 μg/mL, at least 900 μg/mL, or more.

In some embodiments of any of the aspects, the pre-determined concentration of bioproduct in the at least one reactor chamber (e.g., the single reactor chamber or secondary reactor chamber) or production solution in the at least one reactor chamber (e.g., the single reactor chamber or secondary reactor chamber) is at least 1 mg/mL, at least 2 mg/mL, at least 3 mg/mL, at least 4 mg/mL, at least 5 mg/mL, at least 6 mg/mL, at least 7 mg/mL, at least 8 mg/mL, at least 9 mg/mL, at least 10 mg/mL, at least 10 mg/mL, at least 20 mg/mL, at least 30 mg/mL, at least 40 mg/mL, at least 50 mg/mL, at least 60 mg/mL, at least 70 mg/mL, at least 80 mg/mL, at least 90 mg/mL, at least 10⁰ mg/mL, at least 10⁰ mg/mL, at least 200 mg/mL, at least 300 mg/mL, at least 400 mg/mL, at least 500 mg/mL, at least 600 mg/mL, at least 700 mg/mL, at least 800 mg/mL, at least 900 mg/mL, or more.

In some embodiments of any of the aspects, the pre-determined concentration of bioproduct in the at least one reactor chamber (e.g., the single reactor chamber or secondary reactor chamber) or production solution in the at least one reactor chamber (e.g., the single reactor chamber or secondary reactor chamber) is at least 1 g/mL, at least 2 g/mL, at least 3 g/mL, at least 4 g/mL, at least 5 g/mL, at least 6 g/mL, at least 7 g/mL, at least 8 g/mL, at least 9 g/mL, at least 10 g/mL, at least 10 g/mL, at least 20 g/mL, at least 30 g/mL, at least 40 g/mL, at least 50 g/mL, at least 60 g/mL, at least 70 g/mL, at least 80 g/mL, at least 90 g/mL, at least 10⁰ g/mL, at least 10⁰ g/mL, at least 200 g/mL, at least 300 g/mL, at least 400 g/mL, at least 500 g/mL, at least 600 g/mL, at least 700 g/mL, at least 800 g/mL, at least 900 g/mL, or more.

In some embodiments of any of the aspects, the bacterium exhibits a maximum specific growth rate, e.g., in the at least one growth solution and/or at least one production solution. The specific growth rate can be measured as cell per cell per hour or bioproduct per cell per hour. Cell number can be measured using standard techniques, e.g., by OD measurements, or serial dilution and plating. The bioproduct can be measured using standard techniques according to the specific bioproduct. For example, thin layer chromatography (TLC) can be used to detect bioproducts, such as TAGs. The technique for detecting a bioproduct can be selected by a person of skill in the art according to the specific bioproduct (e.g., polypeptide, glycoprotein, lipoprotein, lipid, monosaccharide, polysaccharide, nucleic acid, small molecule, or metabolite).

In some embodiments of any of the aspects, the specific growth rate is at least 0.3 hr⁻¹. In some embodiments of any of the aspects, the specific growth rate is at least 0.21 hr⁻¹. In some embodiments of any of the aspects, the specific growth rate is at least 0.1 hr⁻¹, at least 0.2 hr⁻¹, at least 0.3 hr⁻¹, at least 0.4 hr⁻¹, at least 0.5 hr⁻¹, at least 0.6 hr⁻¹, at least 0.7 hr⁻¹, at least 0.8 hr⁻¹, at least 0.9 hr⁻¹, at least 1.0 hr⁻¹, 0-0.3 hr⁻¹, 0-0.21 hr⁻¹, or 0-0.20 hr⁻¹.

In some embodiments of any of the aspects, the bacterium does not exhibit substantial growth in the production solution (e.g., in the at least one reactor chamber (e.g., the single reactor chamber or at least one secondary reactor chamber)). As used herein, the term “substantial” refers to of ample or considerable amount, quantity, or size as determined by a user. In some embodiments of any of the aspects, the bacterium increases its concentration in the production solution (e.g., in the at least one reactor chamber (e.g., a single reactor chamber or secondary reactor chamber)) by at most 1%, at most 2%, at most 3%, at most 4%, at most 5%, at most 6%, at most 7%, at most 8%, at most 10%, at most 15%, at most 20%, at most 25%, at most 30%, at most 35%, at most 40%, at most 45%, or at most 50%, at most 60%, at most 70%, at most 80%, at most 90%, or at most 10⁰%. In some embodiments of any of the aspects, the growth rate of the bacterium is substantially higher in the growth solution compared to the production solution. As a non-limiting example, the bacterium can undergo 10 doublings in growth in the growth solution compared to 1 doubling in growth in the production solution. In some embodiments of any of the aspects, the growth rate of the bacterium in the growth solution is at least 10× compared to the growth rate of the bacterium in the production solution. In some embodiments of any of the aspects, the growth rate of the bacterium in the growth solution is at least 2×, 3×, 4×5×, 6×, 7×, 8×, 9×, 10×, 11×, 12×, 13×, 14×, 15×, 16×, 17×, 18×, 19×, 20×, or more compared to the growth rate of the bacterium in the production solution.

In some embodiments of any of the aspects, the concentration of the bacterium in the production solution (e.g., in the at least one production solution and/or in the secondary reactor chamber) is at most 10¹ CFU/mL, at most 10² CFU/mL, at most 10³ CFU/mL, at most 10⁴ CFU/mL, at most 10⁵ CFU/mL, at most 10⁶ CFU/mL, at most 10⁷ CFU/mL, at most 10⁸ CFU/mL, at most 10⁹ CFU/mL, at most 10¹⁰ CFU/mL, at most 10¹¹ CFU/mL, or at most 10¹² CFU/mL. In some embodiments of any of the aspects, the concentration of the bacterium in the production solution (e.g., in the at least one production solution and/or in the secondary reactor chamber) is an OD600 measurement of at most 0.1, at most 0.2, at most 0.3, at most 0.4, at most 0.5, at most 0.6, at most 0.7, at most 0.8, at most 0.9, or at most 1.0.

In some embodiments of any of the aspects, the production solution (e.g., second solution in the at least one secondary reactor chamber) comprises: (a) at least 11 molecules of H₂ per molecule of acetyl-CoA: (b) at least ⅓ molecule of organic carbon source and 5/3 molecules of H₂ per molecule of acetyl-CoA; or (c) at least ½ molecule of organic carbon source per molecule of acetyl-CoA. In some embodiments of any of the aspects, the at least one production solution (e.g., the second solution) in the at least one reactor chamber (e.g., the single reactor chamber or at least one secondary reactor chamber) comprises: at least 11 molecules of H₂ per molecule of acetyl-CoA. In some embodiments of any of the aspects, the at least one production solution (e.g., the second solution) in the at least one reactor chamber (e.g., the single reactor chamber or at least one secondary reactor chamber) comprises: at least ⅓ molecule of organic carbon source and 5/3 molecules of H₂ per molecule of acetyl-CoA. In some embodiments of any of the aspects, the at least one production solution (e.g., the second solution) in the at least one reactor chamber (e.g., the single reactor chamber or at least one secondary reactor chamber) comprises at least ½ molecule of organic carbon source per molecule of acetyl-CoA.

In some embodiments of any of the aspects, the at least one production solution (e.g., the second solution) in the at least one reactor chamber (e.g., the single reactor chamber or at least one secondary reactor chamber) comprises: (a) at least 11 molecules of H₂ per molecule of acetyl-CoA; (b) at least 1 molecule of organic carbon source and 5 molecules of H₂ per 3 molecules of acetyl-CoA; or (c) at least 1 molecule of organic carbon source per 2 molecules of acetyl-CoA. In some embodiments of any of the aspects, the at least one production solution (e.g., the second solution) in the at least one reactor chamber (e.g., the single reactor chamber or at least one secondary reactor chamber) comprises at least 11 molecules of H₂ per molecule of acetyl-CoA. In some embodiments of any of the aspects, the at least one production solution (e.g., the second solution) in the at least one reactor chamber (e.g., the single reactor chamber or at least one secondary reactor chamber) comprises at least 1 molecule of organic carbon source and 5 molecules of H₂ per 3 molecules of acetyl-CoA. In some embodiments of any of the aspects, the at least one production solution (e.g., the second solution) in the at least one reactor chamber (e.g., the single reactor chamber or at least one secondary reactor chamber) comprises at least 1 molecule of organic carbon source per 2 molecules of acetyl-CoA.

In some embodiments of any of the aspects, the at least one production solution (e.g., the second solution) in the at least one reactor chamber (e.g., the single reactor chamber or at least one secondary reactor chamber) comprises at least ⅕ molecule, at least ¼ molecule, at least ⅓ molecule, at least ½ molecule, at least 1 molecule, at least 2 molecules, at least 3 molecules, at least 4 molecules, at least 5 molecules, at least 6 molecules, at least 7 molecules, at least 8 molecules, at least 9 molecules, at least 10 molecules, at least 11 molecules, at least 12 molecules, at least 13 molecules, at least 14 molecules, at least 15 molecules, or more of H₂ per molecule of acetyl-CoA.

In some embodiments of any of the aspects, the at least one production solution in the at least one reactor chamber (e.g., the single reactor chamber or at least one secondary reactor chamber) comprises at least ⅕ molecule, at least ¼ molecule, at least ⅓ molecule, at least ½ molecule, at least 1 molecule, at least 2 molecules, at least 3 molecules, at least 4 molecules, at least 5 molecules, at least 6 molecules, at least 7 molecules, at least 8 molecules, at least 9 molecules, at least 10 molecules, at least 11 molecules, at least 12 molecules, at least 13 molecules, at least 14 molecules, at least 15 molecules, or more of organic carbon source per molecule of acetyl-CoA.

In some embodiments of any of the aspects, at least one (e.g., 1, 2, 3, 4, 5, 6, 7, or 8) organic carbon source(s) is selected from the group consisting of: glucose, glycerol, gluconate, acetate, fructose, decanoate, fatty acid, and glycerol gluconate, or any combination thereof. In some embodiments of any of the aspects, the organic carbon source comprises glucose, glycerol, gluconate, acetate, fructose, or decanoate. In some embodiments of any of the aspects, the organic carbon source comprises fatty acid, fructose, glucose, glycerol gluconate, acetate, or decanoate. In some embodiments of any of the aspects, the organic carbon source comprises glucose. In some embodiments of any of the aspects, the organic carbon source comprises glycerol. In some embodiments of any of the aspects, the organic carbon source comprises fructose.

In some embodiments of any of the aspects, the organic carbon source is supplied at a specific rate, e.g., about 20 g/L/hr (e.g., 20.125 g/L/hr). In some embodiments of any of the aspects, the organic carbon source is supplied at least 1 g/L/hr, at least 2 g/L/hr, at least 3 g/L/hr, at least 4 g/L/hr, at least 5 g/L/hr, at least 6 g/L/hr, at least 7 g/L/hr, at least 8 g/L/hr, at least 9 g/L/hr, at least 10 g/L/hr, at least 20 g/L/hr, at least 30 g/L/hr, at least 40 g/L/hr, at least 50 g/L/hr, at least 60 g/L/hr, at least 70 g/L/hr, at least 80 g/L/hr, at least 90 g/L/hr, at least 10⁰ g/L/hr, 0-10 g/L/hr, 1-10 g/L/hr, 10-20 g/L/hr, 20-30 g/L/hr, 15-25 g/L/hr, 0-50 g/L/hr, 1-50 g/L/hr, or 20-100 g/L/hr.

In some embodiments of any of the aspects, the organic carbon source is supplied as a bolus dose. In some embodiments of any of the aspects, the bolus of the organic carbon source comprises about 10 g/L of the organic carbon source. In some embodiments of any of the aspects, the bolus of the organic carbon source comprises about 20 g/L of the organic carbon source. In some embodiments of any of the aspects, the bolus of the organic carbon source comprises at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 20 g/L, at least 30 g/L, at least 40 g/L, at least 50 g/L, at least 60 g/L, at least 70 g/L, at least 80 g/L, at least 90 g/L, at least 10⁰ g/L, 0-10 g/L, 1-10 g/L, 10-20 g/L, 20-30 g/L, 15-25 g/L, 0-50 g/L, 1-50 g/L, or 20-100 g/L.

In some embodiments of any of the aspects, the organic carbon source is supplied throughout the entire run. In some embodiments of any of the aspects, the organic carbon source is supplied during at least a portion of the entire run. In some embodiments of any of the aspects, the organic carbon source is supplied during at least a portion of the culturing the bacterium in the at least one production solution. In some embodiments of any of the aspects, the organic carbon source is supplied during at least a portion of the culturing the bacterium in the at least one growth solution. In some embodiments of any of the aspects, the organic carbon source is supplied (e.g., as a bolus dose) for at least one minute to at most 7 days during the run (e.g., while culturing the bacterium in the at least one growth solution and/or at least one production solution).

In some embodiments of any of the aspects, the organic carbon source is supplied (e.g., as a bolus dose) for at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 20 minutes, at least 30 minutes, at least 40 minutes, at least 50 minutes, at least 60 minutes, at least 70 minutes, at least 80 minutes, at least 90 minutes, at least 10⁰ minutes, at least 110 minutes, at least 120 minutes, at least 3 hours, at least 4 hours, at least 5 hours, at least 6 hours, at least 7 hours, at least 8 hours, at least 9 hours, at least 10 hours, at least 11 hours, at least 12 hours, at least 24 hours, at least 2.5 days, at least 3 days, at least 3.5 days, at least 4 days, at least 4.5 days, at least 5 days, at least 5.5 days, at least 6 days, at least 6.5 days, at least 7 days, at least 8 days, at least 9 days, at least 10 days, at least 11 days, at least 12 days, at least 13 days, at least 14 days, 0-1 weeks, 1-2 weeks, or 0.05-14 days.

In some embodiments of any of the aspects, the cultured bacterium exhibits gas consumption rates of H₂, CO₂, and/or 02. Gas consumption can be measured by analyzing the gas inlet into and gas outlet out of the at least one reactor chamber and then determining a mass balance of the gas. In some embodiments of any of the aspects, the gas consumption rates of H₂, CO₂, and/or 02 is at least 1 mmol/L/hr, at least 2 mmol/L/hr, at least 3 mmol/L/hr, at least 4 mmol/L/hr, at least 5 mmol/L/hr, at least 6 mmol/L/hr, at least 7 mmol/L/hr, at least 8 mmol/L/hr, at least 9 mmol/L/hr, at least 10 mmol/L/hr, at least 20 mmol/L/hr, at least 30 mmol/L/hr, at least 40 mmol/L/hr, at least 50 mmol/L/hr, at least 60 mmol/L/hr, at least 70 mmol/L/hr, at least 80 mmol/L/hr, at least 90 mmol/L/hr, at least 10⁰ mmol/L/hr, at least 110 mmol/L/hr, at least 120 mmol/L/hr, at least 130 mmol/L/hr, at least 140 mmol/L/hr, at least 150 mmol/L/hr, at least 160 mmol/L/hr, at least 170 mmol/L/hr, at least 180 mmol/L/hr, at least 190 mmol/L/hr, at least 200 mmol/L/hr, at least 300 mmol/L/hr, at least 400 mmol/L/hr, at least 500 mmol/L/hr, at least 600 mmol/L/hr, at least 700 mmol/L/hr, at least 800 mmol/L/hr, at least 900 mmol/L/hr, at least 1 mol/L/hr, at least 2 mol/L/hr, at least 3 mol/L/hr, at least 4 mol/L/hr, at least 5 mol/L/hr, at least 6 mol/L/hr, at least 7 mol/L/hr, at least 8 mol/L/hr, at least 9 mol/L/hr, at least 10 mol/L/hr, 0-30 mmol/L/hr, 0-90 mmol/L/hr, 0-170 mmol/L/hr, 0-5 mol/L/hr, or 0-10 mol/L/hr.

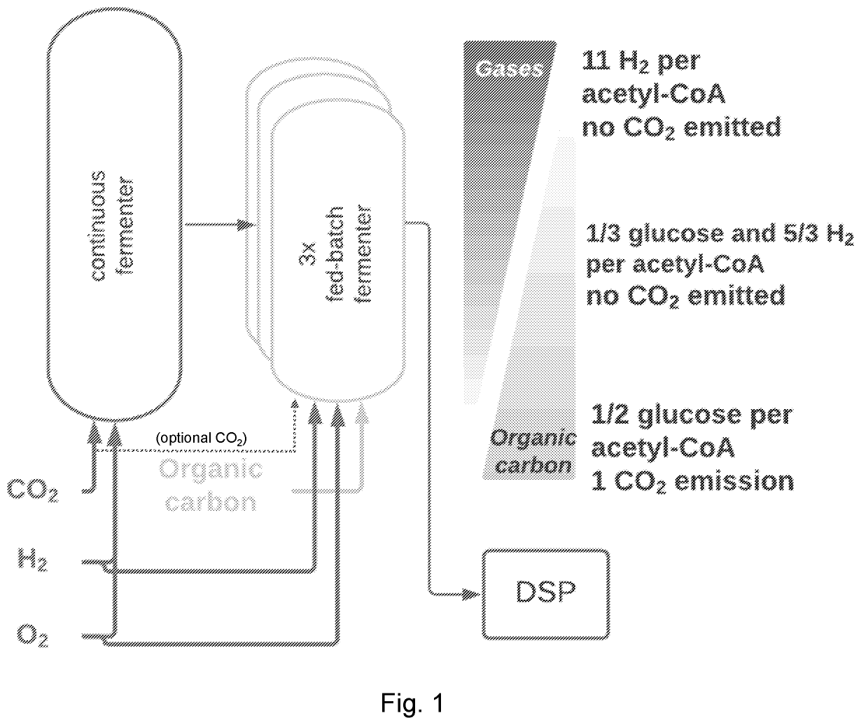

In some embodiments of any of the aspects, the at least one reactor chamber (e.g., a single reactor chamber or primary reactor) chamber emits no CO₂. In some embodiments of any of the aspects, the secondary reactor chamber emits no CO₂. In other words, the fermentation pathways (e.g., gas fermentation, mixotrophic fermentation) used by the microorganisms in the at least one reactor chamber (e.g., a single reactor chamber, or primary and/or secondary reactor chamber(s)) do not produce any CO₂ (see e.g., FIG. 1). In some embodiments of any of the aspects, the at least one reactor chamber (e.g., a single reactor chamber or secondary reactor chamber) emits at most 1 molecule of CO₂ per molecule of acetyl-CoA. In some embodiments of any of the aspects, the at least one reactor chamber (e.g., single reactor chamber or secondary reactor chamber) emits at most 2, at most 3, at most 4, at most 5, at most 6, at most 7, at most 8, at most 9, or at most 10, molecule of CO₂ per molecule of acetyl-CoA.

In some embodiments of any of the aspects, the cells can be maintained in culture. As used herein. “maintaining” refers to continuing the viability of a cell or population of cells. A maintained population of cells will have at least a subpopulation of metabolically active cells.

As used herein, the term “sustainable” refers to a method of harvesting or using a resource so that the resource is not depleted or permanently damaged. In some embodiments of any of the aspects, the resource is a product that is produced by a bacterium as described herein. In some embodiments of any of the aspects, the bacterium sustainably produces bioproducts using a minimal culture medium that comprises CO₂ as the sole carbon source and H₂ as the sole energy source.

As used herein the term “culture medium” refers to a solid, liquid or semi-solid designed to support the growth of microorganisms or cells. In some embodiments of any of the aspects, the culture medium is a liquid. In some embodiments of any of the aspects, the culture medium comprises both the liquid medium and the bacterial cells within it. In some embodiments of any of the aspects, the at least one growth (e.g., first) solution and/or production (e.g., second) solution comprises cell culture medium. In some embodiments of any of the aspects, the at least one growth solution (e.g., the first solution) in at least one reactor chamber (e.g., a single reactor chamber or in a primary reactor chamber) comprises cell culture medium. In some embodiments of any of the aspects, the at least one production solution (e.g., the second solution) in at least one reactor chamber (e.g., in the single reactor chamber or in a secondary reactor chamber) comprises cell culture medium. In some embodiments of any of the aspects, the at least one growth solution and at least one production solution (e.g., first and second solutions) comprises cell culture medium.

In some embodiments of any of the aspects, the culture medium is a defined medium. As used herein, the term “defined medium” refers to a cell culture medium in which all the components and concentrations are known. As a non-limiting example, the defined medium comprises defined levels of specific salts and metal. In some embodiments of any of the aspects, the at least one growth solution and/or production solution (e.g., first and/or second solution) comprises defined medium. In some embodiments of any of the aspects, the at least one growth solution (e.g., the first solution) in at least one reactor chamber (e.g., a single reactor chamber or in a primary reactor chamber) comprises defined medium. In some embodiments of any of the aspects, the at least one production solution (e.g., the second solution) in at least one reactor chamber (e.g., the single reactor chamber or in a secondary reactor chamber) comprises defined medium. In some embodiments of any of the aspects, the at least one growth solution and at least one production solution (e.g., first and second solutions) comprises defined medium.

In some embodiments of any of the aspects, the culture medium is a minimal medium. As used herein, the term “minimal medium” refers to a cell culture medium in which only few and necessary nutrients are supplied, such as a carbon source, a nitrogen source, salts and trace metals dissolved in water with a buffer. Non-limiting examples of components in a minimal medium include Na₂HPO₄ (e.g., 3.5 g/L), KH₂PO₄ (e.g., 1.5 g/L), (NH₄)₂SO₄ (e.g., 1.0 g/L), MgSO₄. 7H₂O (e.g., 80 mg/L), CaSO₄. 2H₂O (e.g., 1 mg/L), NiSO₄. 7H₂O (e.g., 0.56 mg/L), ferric citrate (e.g., 0.4 mg/L), and NaHCO₃ (200 mg/L). In some embodiments of any of the aspects, a minimal medium can be used to promote lithotrophic growth, e.g., of a chemolithotroph. In some embodiments, (NH₄) CI (e.g., 1.0 g/L) is used in addition to or instead of (NH₄)₂SO₂. In some embodiments, the minimal media comprises at least one trace metal from Table 3.

TABLE 3
Exemplary trace metals in culture media; see e.g.,
Mozumder et al., Modeling pure culture heterotrophic
production of polyhydroxybutyrate (PHB), Bioresour
Technol. 2014 March; 155: 272-80.

| | exemplary | |
|---|---|---|
| component | concentration | |
| FeSO₄*7H₂O | 10 | g/L |
| ZnSO₄*7H₂O | 2.25 | g/L |
| CuSO₄*5H₂O | 1 | g/L |
| MnSO₄*5H₂O | 0.5 | g/L |
| 35% HCl | 10 | mL/L |
| CaCl₂*2H₂O | 2 | g/L |
| Na₂B₄O₇*10H₂O | 0.23 | g/L |
| (NH₄)₆Mo₇O₂₄ | 0.1 | g/L |

In some embodiments of any of the aspects, the at least one growth solution and/or at least one production solution (e.g., first and/or second solution) comprises minimal medium. In some embodiments of any of the aspects, the at least one growth solution (e.g., the first solution) in at least one reactor chamber (e.g., a single reactor chamber or in a primary reactor chamber) comprises minimal medium. In some embodiments of any of the aspects, the at least one production solution (e.g., the second solution) in at least one reactor chamber (e.g., the single reactor chamber or in a secondary reactor chamber) comprises minimal medium. In some embodiments of any of the aspects, the at least one growth solution and at least one production solution (e.g., first and second solutions) comprise minimal medium.

In some embodiments of any of the aspects, the culture medium is a rich medium. As used herein, the term “rich medium” refers to a cell culture medium in which more than just a few and necessary nutrients are supplied, e.g., a non-minimal medium. In some embodiments of any of the aspects, rich culture medium can comprise nutrient broth (e.g., 17.5 g/L), yeast extract (7.5 g/L), and/or (NH₄)₂SO₄ (e.g., 5 g/L). In some embodiments of any of the aspects, a rich medium comprises glycerol. In some embodiments of any of the aspects, a rich medium comprises a minimal media, as described herein or known in the art, and additional nutrients (e.g., nutrient broth, yeast extract, etc.). In some embodiments of any of the aspects, a rich medium does necessarily promote lithotrophic growth. In some embodiments of any of the aspects, a rich medium does not necessarily promote lithotrophic growth. In some embodiments of any of the aspects, a rich medium promotes heterotrophic growth.

In some embodiments of any of the aspects, the at least one growth solution and/or at least one production solution (e.g., first and/or second solution) comprises rich medium. In some embodiments of any of the aspects, the at least one growth solution (e.g., the first solution) in at least one reactor chamber (e.g., a single reactor chamber or in a primary reactor chamber) comprises rich medium. In some embodiments of any of the aspects, the at least one production solution (e.g., the second solution) in at least one reactor chamber (e.g., a single reactor chamber or in a secondary reactor chamber) comprises rich medium. In some embodiments of any of the aspects, the at least one growth solution and at least one production solution (e.g., first and second solutions) comprise rich medium.

In some embodiments of any of the aspects, the culture medium, culture vessel, or environment surrounding the culture medium (e.g., in the at least one reactor chamber (e.g., a single reactor chamber, or primary and/or secondary reactor chamber)) or culture vessel (e.g., an incubator) is vented to the outside atmosphere. In some embodiments of any of the aspects, the culture medium, culture vessel, or environment surrounding the culture medium (e.g., in the at least one reactor chamber (e.g., a single reactor chamber, or primary and/or secondary reactor chamber)) or culture vessel (e.g., an incubator) is not vented to the outside atmosphere.

In some embodiments of any of the aspects, the culture medium, culture vessel, or environment surrounding the culture medium (e.g., in the at least one reactor chamber (e.g., a single reactor chamber, or primary and/or secondary reactor chamber)) or culture vessel (e.g., an incubator) comprises 0%-100% H₂, 0%-100% CO₂ and/or 0%-100% O₂. In some embodiments of any of the aspects, the gases in the culture medium, culture vessel, or environment surrounding the culture medium (e.g., in the at least one reactor chamber (e.g., a single reactor chamber, or primary and/or secondary reactor chamber)) or culture vessel (e.g., an incubator) consist of 0%-100% H₂, 0%-100% CO₂ and/or 0%-100% O₂.

In some embodiments of any of the aspects, the culture medium, culture vessel, or environment surrounding the culture medium (e.g., in the at least one reactor chamber (e.g., a single reactor chamber, or primary and/or secondary reactor chamber)) or culture vessel (e.g., an incubator) comprises approximately 68% H₂, approximately 13% CO₂, and/or approximately 19% O₂. In some embodiments of any of the aspects, the gasses in the culture medium, culture vessel, or environment surrounding the culture medium (e.g., in the at least one reactor chamber (e.g., a single reactor chamber, or primary and/or secondary reactor chamber)) or culture vessel (e.g., an incubator) consist of 68% H₂, 13% CO₂, and/or 19% O₂. In some embodiments of any of the aspects, the culture medium, culture vessel, or environment surrounding the culture medium (e.g., in the at least one reactor chamber (e.g., a single reactor chamber, or primary and/or secondary reactor chamber)) or culture vessel (e.g., an incubator) comprises approximately 30% H₂, approximately 15% CO₂, and/or approximately 5% O₂. In some embodiments of any of the aspects, the gasses in the culture medium, culture vessel, or environment surrounding the culture medium (e.g., in the at least one reactor chamber (e.g., a single reactor chamber, or primary and/or secondary reactor chamber) or culture vessel (e.g., an incubator) consist of 30% H₂, 15% CO₂ and/or 5% O₂.

In some embodiments of any of the aspects, the culture medium, culture vessel, or environment surrounding the culture medium (e.g., in the at least one reactor chamber (e.g., a single reactor chamber, or primary and/or secondary reactor chamber)) or culture vessel (e.g., an incubator) comprises at most 10% H₂, at most 20% H₂, at most 30% H₂, at most 40% H₂, at most 50% H₂, at most 60% H₂, at most 61% H₂, at most 62% H₂, at most 63% H₂, at most 64% H₂, at most 65% H₂, at most 66% H₂, at most 67% H₂, at most 68% H₂, at most 69% H₂, at most 70% H₂, at most 80% H₂, at most 90% H₂, at most 95% H₂, at most 99% H₂, or at most 10⁰% H₂. In some embodiments of any of the aspects, the culture medium, culture vessel, or environment surrounding the culture medium (e.g., in the at least one reactor chamber (e.g., a single reactor chamber, or primary and/or secondary reactor chamber)) or culture vessel (e.g., an incubator) comprises at most 5% CO₂, at most 10% CO₂, at most 11% CO₂, at most 12% CO₂, at most 13% CO₂, at most 14% CO₂, at most 15% CO₂, at most 16% CO₂, at most 17% CO₂, at most 18% CO₂, at most 19% CO₂, at most 20% CO₂, at most 25% CO₂, at most 30% CO₂, at most 40% CO₂, at most 50% CO₂, at most 60% CO₂, at most 70% CO₂, at most 80% CO₂, at most 90% CO₂, or at most 10⁰% CO₂. In some embodiments of any of the aspects, the culture medium, culture vessel, or environment surrounding the culture medium (e.g., in the at least one reactor chamber (e.g., a single reactor chamber, or primary and/or secondary reactor chamber)) or culture vessel (e.g., an incubator) comprises at most 1% O₂, at most 2% O₂, at most 3% O₂, at most 4% O₂, at most 5% O₂, at most 10% O₂, at most 11% O₂, at most 12% O₂, at most 13% O₂, at most 14% O₂, at most 15% O₂, at most 16% O₂, at most 17% O₂, at most 18% O₂, at most 19% O₂, at most 20% O₂, at most 25% O₂, at most 30% O₂, at most 40% O₂, at most 50% O₂, at most 60% O₂, at most 70% O₂, at most 80% O₂, at most 90% O₂, or at most 10⁰% O₂.

In some embodiments of any of the aspects, the gases in the culture medium, culture vessel, or environment surrounding the culture medium (e.g., in the at least one reactor chamber (e.g., a single reactor chamber, or primary and/or secondary reactor chamber)) or culture vessel (e.g., an incubator) comprise: at most 1% H₂, at most 2% H₂, at most 3% H₂, at most 4% H₂, at most 5% H₂, at most 6% H₂, 7% H₂, at most 8% H₂, at most 9% H₂, at most 10% H₂, at most 20% H₂, at most 30% H₂, at most 40% H₂, at most 50% H₂, at most 60% H₂, at most 61% H₂, at most 62% H₂, at most 63% H₂, at most 64% H₂, at most 65% H₂, at most 66% H₂, at most 67% H₂, at most 68% H₂, at most 69% H₂, at most 70% H₂, at most 80% H₂, at most 90% H₂, at least 95% H₂, at most 99% H₂, or at most 100% H₂; at most 0.01% CO₂, at most 0.02% CO₂, at most 0.03% CO₂, at most 0.04% CO₂, at most 0.05% CO₂, at most 0.06% CO₂, at most 0.07% CO₂, at most 0.08% CO₂, at most 0.09% CO₂, at most 0.1% CO₂, at most 0.2% CO₂, at most 0.3% CO₂, at most 0.4% CO₂, at most 0.5% CO₂, at most 0.6% CO₂, at most 0.7% CO₂, at most 0.8% CO₂, at most 0.9% CO₂, at most 1% CO₂, at most 2% CO₂, at most 3% CO₂, at most 4% CO₂, at most 5% CO₂, at most 10% CO₂, at most 11% CO₂, at most 12% CO₂, at most 13% CO₂, at most 14% CO₂, at most 15% CO₂, at most 16% CO₂, at most 17% CO₂, at most 18% CO₂, at most 19% CO₂, at most 20% CO₂, at most 25% CO₂, at most 30% CO₂, at most 40% CO₂, at most 50% CO₂, at most 60% CO₂, at most 70% CO₂, at most 80% CO₂, at most 90% CO₂, or at most 100% CO₂; and/or at most 0.01% O₂, at most 0.02% O₂, at most 0.03% O₂, at most 0.04% O₂, at most 0.05% O₂, at most 0.06% O₂, at most 0.07% O₂, at most 0.08% O₂, at most 0.09% O₂, at most 0.1% O₂, at most 0.2% O₂, at most 0.3% O₂, at most 0.4% O₂, at most 0.5% O₂, at most 0.6% O₂, at most 0.7% O₂, at most 0.8% O₂, at most 0.9% O₂, at most 1% O₂, at most 2% O₂, at most 3% O₂, at most 4% O₂, at most 5% O₂, at most 10% O₂, at most 11% O₂, at most 12% O₂, at most 13% O₂, at most 14% O₂, at most 15% O₂, at most 16% O₂, at most 17% O₂, at most 18% O₂, at most 19% O₂, at most 20% O₂, or at most 25% O₂, at most 30% O₂, at most 40% O₂, at most 50% O₂, at most 60% O₂, at most 70% O₂, at most 80% O₂, at most 90% O₂, or at most 10⁰% O₂.

In some embodiments of any of the aspects, the culture medium, culture vessel, or environment surrounding the culture medium (e.g., in the at least one reactor chamber (e.g., a single reactor chamber, or primary and/or secondary reactor chamber)) or culture vessel (e.g., an incubator) is exposed to gas comprising 70%-99.99% atmospheric air with at least one of the following gases added (in addition to such gases already in the atmospheric air); 0.01%-10% H₂, 0.01%-10% CO₂, and/or 0.01%-10% O₂. In some embodiments of any of the aspects, about 3.6% H₂ is added to the atmospheric air. In some embodiments of any of the aspects, about 1.9% CO₂ is added to the atmospheric air. In some embodiments of any of the aspects, about 1.7% CO₂ is added to the atmospheric air.

As used herein, “atmospheric air” refers to air from the environment (e.g., the room or building housing the at least one reactor chamber or culture vessel: e.g., the external environment outside the room or building); atmospheric air can also be referred to as environmental air. By volume, the dry air in Earth's atmosphere is about 78% (e.g., 78.09%) nitrogen, about 21% (e.g., 20.95%) oxygen, about 1% (e.g., 0.93%) argon, and about 0.03 percent trace gases, including but not limited to carbon dioxide, methane, nitrous oxide and ozone.

In some embodiments of any of the aspects, the gas flow rate of at least one gas (e.g., atmospheric air, H₂, CO₂, and/or O₂) into the culture medium, culture vessel, or environment surrounding the culture medium (e.g., in the at least one reactor chamber (e.g., a single reactor chamber, or primary and/or secondary reactor chamber)) or culture vessel (e.g., an incubator) is 0.1 to 5 VVM. The gas flow rate can be measured in VVM, which stands for volume of gas sparged (e.g., in aerobic cultures) per unit volume of growth medium per minute: VVM can be calculated by dividing the measured gas flow rate (e.g., units: L/m, using a rotameter) by the volume (e.g., liters) of growth medium (e.g., including cultured cells). In some embodiments of any of the aspects, the gas flow rate of at least one gas (e.g., atmospheric air, H₂, CO₂, and/or 02) is at least 0.1 VVM, at least 0.2 VVM, at least 0.3 VVM, at least 0.4 VVM, at least 0.5 VVM, at least 0.6 VVM, at least 0.7 VVM, at least 0.8 VVM, at least 0.9 VVM, at least 1 VVM, at least 1.1 VVM, at least 1.2 VVM, at least 1.3 VVM, at least 1.4 VVM, at least 1.5 VVM, at least 1.6 VVM, at least 1.7 VVM, at least 1.8 VVM, at least 1.9 VVM, at least 2 VVM, at least 2.1 VVM, at least 2.2 VVM, at least 2.3 VVM, at least 2.4 VVM, at least 2.5 VVM, at least 2.6 VVM, at least 2.7 VVM, at least 2.8 VVM, at least 2.9 VVM, at least 3 VVM, at least 3.1 VVM, at least 3.2 VVM, at least 3.3 VVM, at least 3.4 VVM, at least 3.5 VVM, at least 3.6 VVM, at least 3.7 VVM, at least 3.8 VVM, at least 3.9 VVM, at least 4 VVM, at least 4.1 VVM, at least 4.2 VVM, at least 4.3 VVM, at least 4.4 VVM, at least 4.5 VVM, at least 4.6 VVM, at least 4.7 VVM, at least 4.8 VVM, at least 4.9 VVM, at least 5 VVM, 0-3 VVM, 0-5 VVM. 0.1-3 VVM, 0.1-5 VVM, 0-1 VVM. 0.1-1 VVM, 1-2 VVM, 2-3 VVM, 3-4 VVM, 4-5 VVM, about 2.1 VVM, or about 2.6 VVM.

In some embodiments of any of the aspects, the culture medium (e.g., in the at least one reactor chamber (e.g., a single reactor chamber, or primary and/or secondary reactor chamber)) comprises CO₂ as the sole carbon source. In some embodiments of any of the aspects, CO₂ is at least 90%, at least 95%, at least 98%, at least 99% or more of the carbon sources present in the culture medium (e.g., in the at least one reactor chamber (e.g., a single reactor chamber, or primary and/or secondary reactor chamber)). In some embodiments of any of the aspects, the culture medium (e.g., in the at least one reactor chamber (e.g., a single reactor chamber, or primary and/or secondary reactor chamber)) comprises CO₂ in the form of bicarbonate (e.g., HCO₃, NaHCO₃) and/or dissolved CO₂ (e.g., atmospheric CO₂; e.g., CO₂ provided by a cell culture incubator). In some embodiments of any of the aspects, the culture medium (e.g., in the at least one reactor chamber (e.g., a single reactor chamber, or primary and/or secondary reactor chamber)) does not comprise organic carbon as a carbon source. Non-limiting example of organic carbon sources include fatty acids, gluconate, acetate, fructose, decanoate, glucose, glycerol, glycerol gluconate: see e.g., Jiang et al. Int J Mol Sci. 2016 July; 17 (7); 1157).

In some embodiments of any of the aspects, the culture medium (e.g., in at least one reactor chamber (e.g., the single reactor chamber or the secondary reactor chamber)) comprises glycerol as the sole carbon source. In some embodiments of any of the aspects, glycerol (e.g., at least one reactor chamber (e.g., the single reactor chamber or the secondary reactor chamber)) is at least 90%, at least 95%, at least 98%, at least 99% or more of the carbon sources present in the culture medium. In some embodiments of any of the aspects, the culture medium (e.g., at least one reactor chamber (e.g., the single reactor chamber or in the secondary reactor chamber)) comprises glycerol and CO₂ as the sole carbon sources. In some embodiments of any of the aspects, the glycerol and CO₂ is at least 90%, at least 95%, at least 98%, at least 99% or more of the carbon sources present in the culture medium (e.g., at least one reactor chamber (e.g., the single reactor chamber or the secondary reactor chamber)).

In some embodiments of any of the aspects, the culture medium (e.g., in the at least one reactor chamber (e.g., a single reactor chamber, or primary and/or secondary reactor chamber)) comprises H₂ as the sole energy source. In some embodiments of any of the aspects, H₂ is at least 90%, at least 95%, at least 98%, at least 99% or more of the energy sources present in the culture medium (e.g., in the at least one reactor chamber (e.g., a single reactor chamber, or primary and/or secondary reactor chamber)). In some embodiments of any of the aspects, H₂ is supplied by water-splitting electrodes in the culture medium (e.g., in the at least one reactor chamber (e.g., a single reactor chamber, or primary and/or secondary reactor chamber)). Accordingly, in one aspect described herein is a system comprising a reactor chamber (e.g., at least one reactor chamber (e.g., a single reactor chamber, or primary and/or secondary reactor chamber)) with a solution (e.g., culture medium) contained therein. The solution may include oxygen (O₂), hydrogen (H₂), carbon dioxide (CO₂), bioavailable nitrogen (e.g., ammonia, (NH₄)₂SO₄, amino acids), and a bacterium as described herein. Gasses such as one or more of hydrogen (H₂), carbon dioxide (CO₂), nitrogen (N₂), and oxygen (O₂) may also be located within a headspace of the reactor chamber, though embodiments in which a reactor does not include a headspace such as in a flow through reactor are also contemplated. The system may also include a pair of electrodes immersed in the solution (e.g., culture medium). The electrodes are configured to apply a voltage potential to, and pass a current through, the solution to split water contained within the culture medium to form at least hydrogen (H₂) and oxygen (O₂) gasses in the solution. These gases may then become dissolved in the solution. During use, a concentration of the bioavailable nitrogen in the solution (e.g., the production solution in the at least one reactor chamber (e.g., the single reactor chamber, or a secondary reactor chamber) may be maintained below a threshold nitrogen concentration that causes the bacteria to produce a desired bioproduct (e.g., triacylglycerides). This product may either by excreted from the bacteria in the at least one reactor chamber (e.g., the single reactor chamber or secondary reactor chamber) and/or stored within the bacteria in the at least one reactor chamber (e.g., the single reactor chamber or secondary reactor chamber) as the disclosure is not so limited (see e.g., US Patent Publication 2018/0265898, the contents of which are incorporated herein by reference in their entirety).

In some embodiments of any of the aspects, the culture medium (e.g., in the at least one reactor chamber (e.g., a single reactor chamber, or primary and/or secondary reactor chamber)) does not comprise oxygen (O₂) gasses in the solution, i.e., the culture is grown under anaerobic conditions. In some embodiments of any of the aspects, the culture medium (e.g., in the at least one reactor chamber (e.g., a single reactor chamber, or primary and/or secondary reactor chamber)) comprises low levels of oxygen (O₂) gasses in the solution, i.e., the culture is grown under hypoxic conditions or microoxic conditions. As a non-limiting example, the culture medium (e.g., in the at least one reactor chamber (e.g., a single reactor chamber, or primary and/or secondary reactor chamber)) can comprise at most 30%, at most 20%, at most 15%, at most 10%, at most 5%, at most 4%, at most 3%, at most 2%, or at most 1% O₂ gasses in the solution.

In some embodiments of any of the aspects, the culture medium (e.g., in the at least one reactor chamber (e.g., the single reactor chamber or the secondary reactor chamber)) further comprises arabinose. In some embodiments of any of the aspects, arabinose acts as an inducer for genes in a pBAD vector. In some embodiments of any of the aspects, the culture medium (e.g., in the at least one reactor chamber (e.g., the single reactor chamber or the secondary reactor chamber)) further comprises at least 0.1% arabinose. As a non-limiting example, the culture medium (e.g., in the at least one reactor chamber (e.g., the single reactor chamber or the secondary reactor chamber)) further comprises at least 0.1% arabinose, at least 0.2% arabinose, at least 0.3% arabinose, at least 0.4% arabinose, at least 0.5% arabinose, 0.6% arabinose, at least 0.7% arabinose, at least 0.8% arabinose, at least 0.9% arabinose, or at least 1.0% arabinose.

In some embodiments of any of the aspects, methods described herein comprise isolating, collecting, or concentrating a product from a bacterium or from the culture medium of a bacterium. In some embodiments of any of the aspects, the bioproduct is isolated, collected, or concentrated (e.g., from the at least one reactor chamber (e.g., the single reactor chamber or the secondary reactor chamber)) after the bacterium produces a pre-determined concentration of the bioproduct. In some embodiments of any of the aspects, after culturing, fermentation and/or bioproduction of or by the bacterium has occurred in the at least one reactor chamber (e.g., the single reactor chamber or at least one secondary reaction chamber), the secondary reaction chamber comprises a metabolized production solution (e.g., an at least partially metabolized production solution or an at least partially metabolized second solution). As compared to the at least one production solution (e.g., the second solution), the metabolized production solution (e.g., metabolized second solution) further comprises bacterium and bioproduct and may optionally include less starting material (e.g., carbon dioxide (CO₂), hydrogen (H₂), oxygen (O₂), organic carbon sources) and more waste products produced by gas fermentation, organic carbon fermentation, and/or mixotrophic fermentation. In some embodiments of any of the aspects, at least a portion of the metabolized production solution (e.g., metabolized second solution) from the at least one secondary reaction chamber is used to isolate, collect, or concentrate the bioproduct.

As used herein the terms “isolate,” “collect,” “concentrate”, “purify” and “extract” are used interchangeably and refer to a process whereby a target component (e.g., TAGs) is removed from a source, such as a fluid (e.g., culture medium). In some embodiments of any of the aspects, methods of isolation, collection, concentration, purification, and/or extraction comprise a reduction in the amount of at least one heterogeneous element (e.g., proteins, nucleic acids: i.e., a contaminant). In some embodiments of any of the aspects, methods of isolation, collection, concentration, purification, and/or extraction reduce by 1%, 2%, 3%, 4%, 5%, 10%, 15%, 20%, 25%, 30%, 35%, 40%, 45%, 50%, 55%, 60%, 65%, 70%, 75%, 80%, 85%, 90% or 95%, or more, the amount of heterogeneous elements, for example biological macromolecules such as proteins or DNA, that may be present in a sample comprising a molecule of interest. The presence of heterogeneous proteins can be assayed by any appropriate method including High-performance Liquid Chromatography (HPLC), gel electrophoresis and staining and/or ELISA assay. The presence of DNA and other nucleic acids can be assayed by any appropriate method including gel electrophoresis and staining and/or assays employing polymerase chain reaction.

Described herein are microorganisms that can be used to sustainably produce bioproducts (e.g., triacylglycerides). In some embodiments of any of the aspects, the microorganism is a bacterium. In some embodiments of any of the aspects, the microorganism is a bacterium, archaea, fungi, plant (e.g., algae), or protist. In some embodiments of any of the aspects, the microorganism is any organism capable of producing a bioproduct in the systems or methods as described herein. In some embodiments of any of the aspects, the microorganism is capable of mixotrophy and/or switchotrophy. It is contemplated herein that any such microorganism (e.g., bacterium, archaea, fungi, plant (e.g., algae), or protist) can be used in place the bacteria described herein. As such, the terms “bacteria” and “microorganism” can be used interchangeably herein, unless the embodiment specifically calls for use of a bacterium.

In some embodiments of any of the aspects, the bacterium naturally produces the bioproduct. In some embodiments of any of the aspects, the bacterium is engineered to sustainably produce bioproducts. Non-limiting examples of bioproducts include polypeptides, glycoproteins, lipoproteins, lipids, monosaccharides, polysaccharides, nucleic acids, small molecules, or metabolites. In some embodiments of any of the aspects, the bioproduct is selected from the group consisting of: polyhydroxyalkanoate (PHA); sucrose: lipochitooligosaccharide; and triacylglyceride.

In some embodiments of any of the aspects, the bacterium is a chemoautotroph. In some embodiments of any of the aspects, the bacterium can grow under chemoautotrophic (i.e., lithotrophic) conditions. As used herein, the term “chemoautotroph” refers to an organism that uses inorganic energy sources to synthesize organic compounds from carbon dioxide. The term “chemolithotroph” can be used interchangeably with chemoautotroph or chemolithoautotroph. Chemoautotrophs stand in contrast to heterotrophs. As used herein, the term “heterotroph” refers to an organism that derives its nutritional requirements from complex organic substances (e.g., sugars).

In some embodiments of any of the aspects, the bacterium is a chemolithotroph. As used herein, the term “chemolithotroph” refers to an organism that is able to use inorganic reduced compounds (e.g., hydrogen, nitrite, iron, sulfur) as a source of energy (e.g., as electron donors). The chemolithotrophy process is accomplished through oxidation of inorganic compounds and ATP synthesis. The majority of chemolithotrophs are able to fix carbon dioxide (CO₂) through the Calvin cycle, a metabolic pathway in which carbon enters as CO₂ and leaves as glucose (see e.g., Kuenen, G. (2009). “Oxidation of Inorganic Compounds by Chemolithotrophs”. In Lengeler, J.; Drews, G.; Schlegel, H. (eds.), Biology of the Prokaryotes. John Wiley & Sons, p. 242. ISBN 9781444313307). The chemolithotroph group of organisms includes sulfur oxidizers, nitrifying bacteria, iron oxidizers, and hydrogen oxidizers. The term “chemolithotrophy” refers to a cell's acquisition of energy from the oxidation of inorganic compounds, also known as electron donors. This form of metabolism is known to occur only in prokaryotes. See e.g., Table 1 for non-limiting examples of chemolithotrophic bacteria and archaea.

TABLE 1
Chemolithotrophic bacteria and archaea

| | Non-Limiting | Source | Respiration |
|---|---|---|---|
| | Examples of | of energy | electron |
| Bacteria | Chemolithotrophs | and electrons | acceptor |
| Iron | Acidithiobacillus | Fe²⁺ (ferrous iron) → | O₂ (oxygen) → |
| bacteria | ferrooxidans | Fe³⁺ (ferric iron) + e⁻ | H₂O (water) |
| Nitrosifying | Nitrosomonas | NH₃ (ammonia) → | O₂ (oxygen) → |
| bacteria | | NO₂⁻ (nitrite) + e⁻ | H₂O (water) |
| Nitrifying | Nitrobacter | NO₂⁻ (nitrite) → | O₂ (oxygen) → |
| bacteria | | NO₃⁻ (nitrate) + e⁻ | H₂O (water) |
| Chemotrophic | Halothiobacillaceae | S²⁻ (sulfide) → S⁰ | O₂ (oxygen) → |
| purple sulfur | | (sulfur) + e⁻ | H₂O (water) |
| bacteria | | | |
| Sulfur- | Chemotrophic | S⁰ (sulfur) → | O₂ (oxygen) → |
| oxidizing | Rhodobacteraceae | SO₄²⁻ (sulfate) + e⁻ | H₂O (water) |
| bacteria | and Thiotrichaceae | | |
| Aerobic | Cupriavidus necator; | H₂ (hydrogen) → H₂O | O₂ (oxygen) → |
| hydrogen | Cupriavidus | (water) + e⁻ | H₂O (water) |
| bacteria | metallidurans | | |
| Anammox | Planctomycetes | NH₄⁺ (ammonium) → NO₂⁻ | N₂ (nitrogen) + |
| bacteria | | (nitrite) | H₂O (water) |
| Thiobacillus | Thiobacillus | S⁰ (sulfur) → | NO₃⁻ (nitrate) |
| denitrificans | denitrificans | SO₄²⁻ (sulfate) + e⁻ | |
| Sulfate-reducing | Desulfovibrio | H₂ (hydrogen) → H₂O | Sulfate |
| bacteria: Hydrogen | paquesii | (water) + e⁻ | (SO₄²⁻) |
| bacteria | | | |
| Sulfate-reducing | Desulfotignum | PO₃³⁻ (phosphite) → PO₄³⁻ | Sulfate |
| bacteria: Phosphite | phosphitoxidans | (phosphate) + e⁻ | (SO₄²⁻) |
| bacteria | | | |
| Methanogens | Archaea | H₂ (hydrogen) → H₂O | CO₂ (carbon |
| | | (water) + e⁻ | dioxide) |
| Carboxydotrophic | Carboxydothermus | carbon monoxide (CO) → | H₂O (water) → |
| bacteria | hydrogenoformans | carbon dioxide | H₂ (hydrogen) |
| | | (CO₂) + e⁻ | |

In some embodiments of any of the aspects, the bacterium is a chemolithotroph belonging to a classification selected from the group consisting of Acidithiobacillus, Alcaligenes, Carboxydothermus, Cupriavidus, Desulfotignum, Desulfovibrio, Halothiobacillaceae, Hydrogenomonas, Nitrobacter, Nitrosomonas, Planctomycetes, Ralstonia, Rhodobacteraceae, Thiobacillus, Thiotrichaceae, and Wautersia. In some embodiments of any of the aspects, the microorganism is a methanogenic archaea (e.g., belonging to the genera Methanosarcina or Methanothrix). In some embodiments of any of the aspects, the bacterium is selected from the group consisting of Acidithiobacillus ferrooxidans, Carboxydothermus hydrogenoformans, Cupriavidus metallidurans, Cupriavidus necator, Desulfotignum phosphitoxidans, Desulfovibrio paquesii, and Thiobacillus denitrificans. In some embodiments of any of the aspects, the bacterium is further engineered to be chemolithotrophic. In some embodiments of any of the aspects, the bacterium is aerobic and uses O₂ as its respiration electron acceptor. In some embodiments of any of the aspects, the bacteria can be a heterotroph or a chemolithotroph, e.g., depending on environmental conditions.

In some embodiments of any of the aspects, the bacterium is a mixotroph. As used herein the term “mixotroph” refers to an organism capable of functioning as both autotrophy (e.g., chemolithotrophy) and heterotroph. As a non-limiting example, a mixotroph is capable of both gas fermentation (autotrophy) and organic carbon (e.g., sugar) fermentation (heterotrophy). In some embodiments of any of the aspects, the mixotroph belongs to the Cupriavidus genus. In some embodiments of any of the aspects, the mixotroph is C. necator. In some embodiments of any of the aspects, the mixotroph is Clostridium ljungdahlii or Clostridium autoethanogenum. In some embodiments of any of the aspects, the mixotroph is selected from the group consisting of: Chloroflexi, Cyanobacteria, and Proteobacteria. In some embodiments of any of the aspects, the mixotroph belongs to the Rhodococcus genus. In some embodiments of any of the aspects, the mixotroph is Rhodococcus opacus or Rhodococcus sp.

In some embodiments of any of the aspects, the bacterium is a switchotroph. As used herein the term “switchotroph” refers to an organism capable of switching between autotrophy (e.g., chemolithotrophy) and heterotrophy. As a non-limiting example, a switchotroph is capable of switching between gas fermentation (autotrophy) and organic carbon (e.g., sugar) fermentation (heterotrophy). In some embodiments of any of the aspects, the switchotroph belongs to the Cupriavidus genus. In some embodiments of any of the aspects, the switchotroph is C. necator. In some embodiments of any of the aspects, the switchotroph belongs to the Rhodococcus genus. In some embodiments of any of the aspects, the switchotroph is Rhodococcus opacus or Rhodococcus sp.

In some embodiments of any of the aspects, the bacterium is not a heterotroph. As used herein the term “heterotroph” refers to an organism that is only capable of organic carbon fermentation, and is not an autotroph, chemolithotroph, mixotroph, or switchotroph. Heterotrophs are not capable of gas fermentation or mixotrophic fermentation or switching between gas fermentation and organic carbon fermentation. The systems and methods described herein are specifically contemplated for use with autotrophs or chemolithotrophs, mixotrophs, or switchotrophs, but not for use with heterotrophs.

In some embodiments of any of the aspects, the bacterium uses CO₂ as its sole carbon source or H₂ as its sole energy source. In some embodiments of any of the aspects, the bacterium uses CO₂ as its sole carbon source and H₂ as its sole energy source. In some embodiments of any of the aspects, the bacterium uses H₂ as its sole energy source. In some embodiments of any of the aspects, the bacterium uses CO₂ as its sole carbon source.

In some embodiments of any of the aspects, the bacterium is engineered from a bacterium that uses CO₂ as its sole carbon source or H₂ as its sole energy source. In some embodiments of any of the aspects, the bacterium is engineered from a bacterium that uses CO₂ as its sole carbon source and H₂ as its sole energy source. In some embodiments of any of the aspects, the bacterium is engineered from a bacterium that uses H₂ as its sole energy source. In some embodiments of any of the aspects, the bacterium is engineered from a bacterium that uses CO₂ as its sole carbon source. In some embodiments of any of the aspects, the bacterium is engineered from a mixotroph. In some embodiments of any of the aspects, the bacterium is engineered from a switchotroph.

In some embodiments of any of the aspects, the bacterium obtains at least 90%, at least 95%, at least 98%, at least 99% or more of its carbon from CO₂ (e.g., in the at least one reactor chamber (e.g., a single reactor chamber, or primary and/or secondary reactor chamber)). In some embodiments of any of the aspects, the bacterium obtains at least 90%, at least 95%, at least 98%, at least 99% or more of its energy from H₂. In some embodiments of any of the aspects, the bacterium obtains at least 90%, at least 95%, at least 98%, at least 99% or more of its carbon from CO₂ and at least 90%, at least 95%, at least 98%, at least 99% or more of its energy from H₂ (e.g., in the at least one reactor chamber (e.g., a single reactor chamber, or primary and/or secondary reactor chamber)).

As used herein, the term “carbon source” refers to the molecules used by an organism as the source of carbon for building its biomass: a carbon source can be an organic compound or an inorganic compound. “Source” denotes an environmental source. In some embodiments of any of the aspects, the bacterium fixes carbon dioxide (CO₂) through the Calvin cycle, a metabolic pathway in which carbon enters as CO₂ and leaves as glucose. As used herein, the term “sole carbon source” denotes that the bacterium uses only the indicated carbon source (e.g., CO₂) and no other carbon sources. For example, “sole carbon source” is intended to mean where the suitable conditions comprise a culture media containing a carbon source such that, as a fraction of the total carbon atoms in the media, the specific carbon source (e.g., CO₂), respectively, represent about 100% of the total carbon atoms in the media. In some embodiments, the sole carbon source of the bacteria is inorganic carbon, including but not limited to carbon dioxide (CO₂) and bicarbonate (HCO₃). In some embodiments of any of the aspects, the sole carbon source is atmospheric CO₂. In some embodiments, the bacterium uses a first sole carbon source (e.g., CO₂; e.g., CO₂ and an organic carbon source) in the growth solution and at least a second carbon source (e.g., CO₂; CO₂ and an organic carbon source; or an organic carbon source) in the production solution. In some embodiments, the bacterium uses a first sole carbon source (e.g., CO₂; e.g., CO₂ and an organic carbon source) in the primary reactor chamber and at least a second carbon source (e.g., CO₂; CO₂ and an organic carbon source; or an organic carbon source) in the secondary reactor chamber.

In some embodiments of any of the aspects, the bacterium uses CO₂ as its major carbon source (e.g., in the at least one reactor chamber (e.g., a single reactor chamber, or primary and/or secondary reactor chamber)), meaning at least 50% of its carbon atoms are obtained from CO₂. As a non-limiting example, the bacterium obtains at least 50%, at least 60%, at least 70%, at least 80%, at least 90%, at least 95%, or at least 99% of its carbon atoms from CO₂ (e.g., in the at least one reactor chamber (e.g., a single reactor chamber, or primary and/or secondary reactor chamber)).

In some embodiments of any of the aspects, the bacterium does not use organic carbon as a carbon source (e.g., in the at least one reactor chamber (e.g., a single reactor chamber, or primary and/or secondary reactor chamber)). Non-limiting example of organic carbon sources include fatty acids, gluconate, acetate, fructose, decanoate: see e.g., Jiang et al. Int J Mol Sci. 2016 July: 17 (7); 1157).

In some embodiments of any of the aspects, the bacterium uses a simple organic carbon source as its sole carbon source (e.g., in the at least one reactor chamber (e.g., a single reactor chamber, or secondary reactor chamber)). Non-limiting examples of simple organic carbon sources include: glucose, glycerol, gluconate, acetate, fructose, or decanoate. In some embodiments of any of the aspects, the bacterium uses fructose as its sole carbon source (e.g., in the at least one reactor chamber (e.g., a single reactor chamber, or secondary reactor chamber)). In some embodiments of any of the aspects, the bacterium uses fructose and CO₂ as its carbon sources (e.g., in the at least one reactor chamber (e.g., a single reactor chamber, or secondary reactor chamber)). In some embodiments of any of the aspects, the bacterium is engineered from a bacterium that uses fructose as its sole carbon source. In some embodiments of any of the aspects, the bacterium obtains at least 90%, at least 95%, at least 98%, at least 99% or more of its carbon from fructose. In some embodiments of any of the aspects, the bacterium uses fructose as its major carbon source, meaning at least 50% of its carbon atoms are obtained from fructose (e.g., in the at least one reactor chamber (e.g., a single reactor chamber, or secondary reactor chamber)). As a non-limiting example, the bacterium obtains at least 50%, at least 60%, at least 70%, at least 80%, at least 90%, at least 95%, or at least 99% of its carbon atoms from fructose (e.g., in the secondary reactor chamber).

In some embodiments of any of the aspects, the bacterium uses glycerol as its sole carbon source (e.g., in the at least one reactor chamber (e.g., a single reactor chamber, or secondary reactor chamber)). In some embodiments of any of the aspects, the bacterium uses glycerol and CO₂ as its carbon sources (e.g., in the at least one reactor chamber (e.g., a single reactor chamber, or secondary reactor chamber)). In some embodiments of any of the aspects, the bacterium is engineered from a bacterium that uses glycerol as its sole carbon source. In some embodiments of any of the aspects, the bacterium obtains at least 90%, at least 95%, at least 98%, at least 99% or more of its carbon from glycerol. In some embodiments of any of the aspects, the bacterium uses glycerol as its major carbon source, meaning at least 50% of its carbon atoms are obtained from glycerol (e.g., in the secondary reactor chamber). As a non-limiting example, the bacteria obtains at least 50%, at least 60%, at least 70%, at least 80%, at least 90%, at least 95%, or at least 99% of its carbon atoms from glycerol (e.g., in the secondary reactor chamber).

In some embodiments of any of the aspects, the bacterium uses H₂ as its sole energy source (e.g., in the at least one reactor chamber (e.g., a single reactor chamber, or primary and/or secondary reactor chamber)). As used herein, the term “energy source” refers to molecules that contribute electrons and contribute to the process of ATP synthesis. As described here, the bacterium can be a chemolithotroph, i.e., an organism that is able to use inorganic reduced compounds (e.g., hydrogen, nitrite, iron, sulfur) as a source of energy (e.g., as electron donors). As used herein, the term “sole energy source” denotes that the bacterium uses only the indicated energy source (e.g., H₂) and no other energy sources. In some embodiments of any of the aspects, the sole energy source is atmospheric H₂ (e.g., in the at least one reactor chamber (e.g., a single reactor chamber, or primary and/or secondary reactor chamber)). In some embodiments of any of the aspects, H₂ is supplied by electrodes in the solution (e.g., in the at least one reactor chamber (e.g., a single reactor chamber, or primary and/or secondary reactor chamber)).

In some embodiments of any of the aspects, the bacterium uses H₂ as its major energy source, meaning at least 50% of its donated electrons (e.g., used for ATP synthesis) are obtained from H₂ (e.g., in the at least one reactor chamber (e.g., a single reactor chamber, or primary and/or secondary reactor chamber)). As a non-limiting example, the bacterium obtains at least 50%, at least 60%, at least 70%, at least 80%, at least 90%, at least 95%, or at least 99% of its donated electrons from H₂ (e.g., in the at least one reactor chamber (e.g., a single reactor chamber, or primary and/or secondary reactor chamber)).

Bacteria used in the systems and methods disclosed herein may be selected so that the bacteria both oxidize hydrogen as well as consume carbon dioxide. Accordingly, in some embodiments, the bacteria may include an enzyme capable of metabolizing hydrogen as an energy source such as with hydrogenase enzymes. Additionally, the bacteria may include one or more enzymes capable of performing carbon fixation such as Ribulose-1,5-bisphosphate carboxy lase/oxygenase (RuBisCO). One possible class of bacteria that may be used in the systems and methods described herein to produce a product include, but are not limited to, chemolithoautotrophs. Additionally, appropriate chemolithoautotrophs may include any one or more of Ralstonia eutropha (R. eutropha) as well as Alcaligenes paradoxs I 360 bacteria, Alcaligenes paradoxs 12/X bacteria, Nocardia opaca bacteria, Nocardia autotrophica bacteria, Paracoccus denitrificans bacteria, Pseudomonas facilis bacteria, Arthrobacter species 11× bacteria, Xanthobacter autotrophicus bacteria, Azospirillum lipferum bacteria, Derxia gummosa bacteria, Rhizobium japonicum bacteria, Microcyclus aquaticus bacteria, Microcyclus ebruneus bacteria, Renobacter vacuolatum bacteria, and any other appropriate bacteria.

In some embodiments of any of the aspects, the bacterium belongs to the Cupriavidus genus. The Cupriavidus genus of bacteria includes the former genus Wautersia. Cupriavidus bacteria are characterized as Gram-negative, motile, rod-shaped organisms with oxidative metabolism. Cupriavidus bacteria possess peritrichous flagella, are obligate aerobic organisms, and are chemoorganotrophic or chemolithotrophic. In some embodiments of any of the aspects, the bacteria is selected from the group consisting of Cupriavidus alkaliphilus, Cupriavidus basilensis, Cupriavidus campinensis, Cupriavidus gilardii, Cupriavidus laharis, Cupriavidus metallidurans, Cupriavidus necator, Cupriavidus nantongensis, Cupriavidus numazuensis, Cupriavidus oxalaticus, Cupriavidus pampae, Cupriavidus pauculus, Cupriavidus pinatubonensis, Cupriavidus plantarum, Cupriavidus respiraculi, Cupriavidus taiwanensis, and Cupriavidus yeoncheonensis.

In some embodiments of any of the aspects, the bacterium is Cupriavidus necator. Cupriavidus necator can also be referred to as Ralstonia eutropha, Hydrogenomonas eutrophus, Alcaligenes eutropha, or Wautersia eutropha. In some embodiments of any of the aspects, the bacterium is Cupriavidus necator strain H₁₆. In some embodiments of any of the aspects, the bacterium is Cupriavidus necator strain N-1.

Members of the species and genera described herein can be identified genetically and/or phenotypically. By way of non-limiting example, the bacterium as described herein comprises a 16S rDNA sequence at least 97% identical to a 16S rDNA sequence present in a reference strain operational taxonomic unit for Cupriavidus necator. In some embodiments of any of the aspects, the bacterium as described herein comprises a 16S rDNA that comprises SEQ ID NO: 1 or SEQ ID NO: 2 or a sequences that is at least 95%, at least 96%, at least 97%, at least 98%, or at least 99% identical to the sequence of SEQ ID NO: 1 or SEQ ID NO: 2. In some embodiments of any of the aspects, the bacterium as described herein is Cupriavidus necator (e.g., strain H₁₆ or strain N-1).

Cupriavidus necator strain N-1 16S
ribosomal RNA, partial sequence,
NCBI Reference Sequence: NR_028766.1,
1356 nucleotides (nt)
SEQ ID NO: 1
TTAGATTGAACGCTGGCGGCATGCCTTACACATGCAAGTCGAACG
GCAGCACGGGCTTCGGCCTGGTGGCGAGTGGCGAACGGGTGAGTA
ATACATCGGAACGTGCCCTGTAGTGGGGGATAACTAGTCGAAAGA
TTAGCTAATACCGCATACGACCTGAGGGTGAAAGCGGGGGACCGC
AAGGCCTCGCGCTACAGGAGCGGCCGATGTCTGATTAGCTAGTTG
GTGGGGTAAAAGCCTACCAAGGCGACGATCAGTAGCTGGTCTGAG
AGGACGATCAGCCACACTGGGACTGAGACACGGCCCAGACTCCTA
CGGGAGGCAGCAGTGGGGAATTTTGGACAATGGGGGCAACCCTGA
TCCAGCAATGCCGCGTGTGTGAAGAAGGCCTTCGGGTTGTAAAGC
ACTTTTGTCCGGAAAGAAATGGCTCTGGTTAATACCCGGGGTCGA
TGACGGTACCGGAAGAATAAGCACCGGCTAACTACGTGCCAGCAG
CCGCGGTAATACGTAGGGTGCGAGCGTTAATCGGAATTACTGGGC
GTAAAGCGTGCGCAGGCGGTTTTGTAAGACAGGCGTGAAATCCCC
GAGCTCAACTTGGGAATGGCGCTTGTGACTGCAAGGCTAGAGTAT
GTCAGAGGGGGGTAGAATTCCACGTGTAGCAGTGAAATGCGTAGA
GATGTGGAGGAATACCGATGGCGAAGGCAGCCCCCTGGGACGTCA
CTGACGCTCATGCACGAAAGCGTGGGGAGCAAACAGGATTAGATA
CCCTGGTAGTCCACGCCCTAAACGATGTCAACTAGTTGTTGGGGA
TTCATTTCTTCAGTAACGTAGCTAACGCGTGAAGTTGACCGCCTG
GGGAGTACGGTCGCAAGATTAAAACTCAAAGGAATTGACGGGGAC
CCGCACAAGCGGTGGATGATGTGGATTAATTCGATGCAACGCGAA
AAACCTTACCTACCCTTGACATGCCACTAACGAAGCAGAGATGCA
TTAGGTGCCCGAAAGGGAAAGTGGACACAGGTGCTGCATGGCTGT
CGTCAGCTCGTGTCGTGAGATGTTGGGTTAAGTCCCGCAACGAGC
GCAACCCTTGTCTCTAGTTGCTACGAAAGGGCACTCTAGAGAGAC
TGCCGGTGACAAACCGGAGGAAGGTGGGGATGACGTCAAGTCCTC
ATGGCCCTTATGGGTAGGGCTTCACACGTCATACAATGGTGCGTA
CAGAGGGTTGCCAACCCGCGAGGGGGAGCTAATCCCAGAAAACGC
ATCGTAGTCCGGATCGTAGTCTGCAACTCGACTACGTGAAGCTGG
AATCGCTAGTAATCGCGGATCAGCATGCCGCGGTGAATACGTTCC
CGGTCT,
Cupriavidus necator strain H16 16S
ribosomal RNA, 1537 nt
SEQ ID NO: 2
AGATTGAACTGAAGAGTTTGATCCTGGCTCAGATTGAACGCTGGC
GGCATGCCTTACACATGCAAGTCGAACGGCAGCACGGGCTTCGGC
CTGGTGGCGAGTGGCGAACGGGTGAGTAATACATCGGAACGTGCC
CTGTAGTGGGGGATAACTAGTCGAAAGATTAGCTAATACCGCATA
CGACCTGAGGGTGAAAGCGGGGGACCGCAAGGCCTCGCGCTACAG
GAGCGGCCGATGTCTGATTAGCTAGTTGGTGGGGTAAAAGCCTAC
CAAGGCGACGATCAGTAGCTGGTCTGAGAGGACGATCAGCCACAC
TGGGACTGAGACACGGCCCAGACTCCTACGGGAGGCAGCAGTGGG
GAATTTTGGACAATGGGGGCAACCCTGATCCAGCAATGCCGCGTG
TGTGAAGAAGGCCTTCGGGTTGTAAAGCACTTTTGTCCGGAAAGA
AATGGCTCTGGTTAATACCCGGGGTCGATGACGGTACCGGAAGAA
TAAGCACCGGCTAACTACGTGCCAGCAGCCGCGGTAATACGTAGG
GTGCGAGCGTTAATCGGAATTACTGGGCGTAAAGCGTGCGCAGGC
GGTTTTGTAAGACAGGCGTGAAATCCCCGAGCTCAACTTGGGAAT
GGCGCTTGTGACTGCAAGGCTAGAGTATGTCAGAGGGGGAAGAAT
TCCACGTGTAGCAGTGAAATGCGTAGAGATGTGGAGGAATACCGA
TGGCGAAGGCAGCCCCCTGGGACGTCACTGACGCTCATGCACGAA
AGCGTGGGGAGCAAACAGGATTAGATACCCTGGTAGTCCACGCCC
TAAACGATGTCAACTAGTTGTTGGGGATTCATTTCTTCAGTAACG
TAGCTAACGCGTGAAGTTGACCGCCTGGGGAGTACGGTCGCAAGA
TTAAAACTCAAAGGAATTGACGGGGACCCGCACAAGCGGTGGATG
ATGTGGATTAATTCGATGCAACGCGAAAAACCTTACCTACCCTTG
ACATGCCACTAACGAAGCAGAGATGCATTAGGTGCCCGAAAGGGA
AAGTGGACACAGGTGCTGCATGGCTGTCGTCAGCTCGTGTCGTGA
GATGTTGGGTTAAGTCCCGCAACGAGCGCAACCCTTGTCTCTAGT
TGCTACGAAAGGGCACTCTAGAGAGACTGCCGGTGACAAACCGGA
GGAAGGTGGGGATGACGTCAAGTCCTCATGGCCCTTATGGGTAGG
GCTTCACACGTCATACAATGGTGCGTACAGAGGGTTGCCAACCCG
CGAGGGGGAGCTAATCCCAGAAAACGCATCGTAGTCCGGATCGTA
GTCTGCAACTCGACTACGTGAAGCTGGAATCGCTAGTAATCGCGG
ATCAGCATGCCGCGGTGAATACGTTCCCGGGTCTTGTACACACCG
CCCGTCACACCATGGGAGTGGGTTTTGCCAGAAGTAGTTAGCCTA
ACCGCAAGGAGGGCGATTACCACGGCAGGGTTCATGACTGGGGTG
AAGTCGTAACAAGGTAGCCGTATCGGAAGGTGCGGCTGGATCACC
TCCTTTC,

In some embodiments of any of the aspects, the bacterium comprises at least one engineered inactivating modification of at least one endogenous gene. In some embodiments of any of the aspects, an engineered inactivating modification of an endogenous gene comprises one or more of: i) deletion of the entire coding sequence, ii) deletion of the promoter of the gene, iii) a frameshift mutation, iv) a nonsense mutation (i.e., a premature termination codon), v) a point mutation, vi) a deletion, vii) or an insertion. Non-limiting examples of inactivating modifications include a mutation that decreases gene or polypeptide expression, a mutation that decreases gene or polypeptide transport, a mutation that decreases gene or polypeptide activity, a mutation in the active site of an enzyme that decreases enzymatic activity, or a mutation that decreases the stability of a nucleic acid or polypeptide. Examples of loss-of-function mutations for each gene can be clear to a person of ordinary skill (e.g., a premature stop codon, a frameshift mutation); they can be measurable by an assay of nucleic acid or protein function, activity, expression, transport, and/or stability; or they can be known in the art.

In some embodiments of any of the aspects, an inactivating modification of an endogenous gene can be engineered in a bacterium using an integration vector (e.g., pT18mobsacB). In some embodiments of any of the aspects, the engineering of an inactivating modification of an endogenous gene in a bacterium further comprises conjugation methods and/or counterselection methods. In some embodiments of any of the aspects, the introduction of an integration vector comprising an endogenous gene comprising an inactivating modification causes the endogenous gene to be replaced with the endogenous gene comprising an inactivating modification.

In some embodiments of any of the aspects, the bacterium is engineered to comprise at least one overexpressed gene. In some embodiments of any of the aspects, the overexpressed gene is endogenous. In some embodiments of any of the aspects, the overexpressed gene is exogenous. In some embodiments of any of the aspects, the overexpressed gene is heterologous. In some embodiments of any of the aspects, a gene can be overexpressed using an expression vector (e.g., pBAD, pCR2.1).

In some embodiments of any of the aspects, the bacterium is engineered to comprise at least one exogenous copy of a functional gene. As a non-limiting example, the bacterium can comprise 1, 2, 3, 4, or at least 5 exogenous copies of a functional gene. As used herein, the term “functional” refers to a form of a molecule which possesses either the native biological activity of the naturally existing molecule of its type, or any specific desired activity, for example as judged by its ability to bind to ligand molecules. In some embodiments of any of the aspects, a molecule can comprise at least 50%, at least 60%, at least 70%, at least 75%, at least 80%, at least 85%, at least 90%, at least 95%, at least 96%, at least 97%, at least 98%, at least 99%, or at least 99% of the activity of the wild-type molecule, e.g., in its native organism.

In some embodiments of any of the aspects, a functional gene as described herein is exogenous. In some embodiments of any of the aspects, a functional gene as described herein is ectopic. In some embodiments of any of the aspects, a functional gene as described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism, in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

In some embodiments of any of the aspects, the bacterium is engineered to comprise at least one functional heterologous gene. As used herein, the term “heterologous” refers to that which is not endogenous to, or naturally occurring in, a referenced sequence, molecule (including e.g., a protein), virus, cell, tissue, or organism. For example, a heterologous sequence of the present disclosure can be derived from a different species, or from the same species but substantially modified from an original form. Also for example, a nucleic acid sequence that is not normally expressed in a virus or a cell is a heterologous nucleic acid sequence. The term “heterologous” can refer to DNA, RNA, or protein that does not occur naturally as part of the organism in which it is present or which is found in a location or locations in the genome that differ from that in which it occurs in nature. It is DNA, RNA, or protein that is not endogenous to the virus or cell and has been artificially introduced into the virus or cell.

In some embodiments of any of the aspects, at least one exogenous copy of a functional gene can be engineered into a bacterium using an expression vector (e.g., pBadT). In some embodiments of any of the aspects, the expression vector (e.g., pBadT) is translocated from a donor bacterium (e.g., MFDpir) into the bacterium under conditions that promote conjugation.

In some embodiments of any of the aspects, at least one exogenous or heterologous gene as described herein can comprise a detectable label, including but not limited to c-Myc, HA, VSV-G, HSV, FLAG, V5, HIS, or biotin. Detectable labels can also include, but are not limited to, radioisotopes, bioluminescent compounds, chromophores, antibodies, chemiluminescent compounds, fluorescent compounds, metal chelates, and enzymes.

In some embodiments of any of the aspects, the bacterium further comprises a selectable marker. Non-limiting examples of selectable markers include a positive selection marker: a negative selection marker: a positive and negative selection marker: resistance to at least one of ampicillin, kanamycin, triclosan, and/or chloramphenicol; or an auxotrophy marker. In some embodiments of any of the aspects, the selectable marker is selected from the group consisting of beta-lactamase, Neo gene (e.g., Kanamycin resistance cassette) from Tn5, mutant FabI gene, and an auxotrophic mutation.

In some embodiments of any of the aspects, a bacterium that is resistant to reactive oxygen species may be used. Further, in some embodiments a R. eutropha bacteria that is resistant to ROS as compared to a wild-type H₁₆ R. eutropha may be used. US 2018/0265898 and Table 2 below detail several genetic polymorphisms found between the wild-type H₁₆ R. eutropha and a ROS-tolerant BC4 strain that was purposefully evolved. Mutations of the BC4 strain relative to the wild type bacteria are detailed further below.

TABLE 2
Mutations in ROS-tolerant BC4 strain

| Mutation | Position | Annotation | Gene | Description |
|---|---|---|---|---|
| G → T | 611,894 | R133R | acrC1 | cation/multidrug |
| | | | | efflux system outer |
| | | | | membrane protein |
| Δ45 bp | 611,905 | 344-388 of | acrC1 | cation/multidrug |
| | | 1494 nt | | efflux system outer |
| | | | | membrane protein |
| G → A | 2,563,281 | intergenic, | Hfq and | uncharacterized host |
| | | (−1/+210) | H16_A2360 | factor I protein/ |
| | | | | GTP-binding |
| | | | | protein |
| Δ15 bp | 241,880 | 363-377 of | H16_B0214 | transcriptional |
| | | 957 nt | | regulator, |
| | | | | LysR-Family |

Two single nucleotide polymorphisms and two deletion events have been observed. Without wishing to be bound by theory, the large deletion from acrCl may indicate a decrease in overall membrane permeability, possibly affecting superoxide entry to the cell resulting in the observed ROS resistance. The genome sequences are accessible at the NCBI SRA database under the accession number SRP073266 and specific mutations of the BC4 strain are listed below. The standard genome sequence for the wild-type H16 R. eutropha is also accessible at the RCSB Protein Data Bank under accession number AM260479 which the following mutations may also be referenced to.

In reference to the above table, an R. eutropha bacteria may include at least one to four mutations selected from the mutations noted above in Table 2 and may be selected in any combination. These specific mutations are listed below in more detail with mutations noted relative to the wild type R. eutropha bolded and underlined within the sequences given below.

The first noted mutation may correspond to the sequence listed below ranging from position 611790-611998 for Ralstonia eutropha H16 chromosome 1. The bolded, double underlined text indicates a mutation (e.g., nt 105 of SEQ ID NO: 3).

(209 nt)
SEQ ID NO: 3
GCCTCGCTGCTTTCCACCTGGCGCCGCACGCGGCCCCAGACGTCG
ATTTCCCAGGTTGCGCCCAGGGTCGCGCTCTGCCCGTTGAGCGTG
CTGCCG CTGGCGCC GCGCGCGCGCGAGGCGCCGGCCTGTGCGT
CGACGGTCGGGAAGAAGCCGGCGCGCGCGGCCTGCAGCGACGCCA
CCGCCTGGCGGTACTGCGCCTCGGCGGCCTT

The second noted mutation may correspond to the sequence listed below ranging from position 611905-613399 for Ralstonia eutropha H16 chromosome 1. The bolded, double underlined text indicates a mutation (e.g., nt 345-390 of SEQ ID NO: 4).

(1495 nt)
SEQ ID NO: 4
AGGCGCCGGCCTGTGCGTCGACGGTCGGGAAGAAGCCGGCGCGCG
CGGCCTGCAGCGACGCCACCGCCTGGCGGTACTGCGCCTCGGCGG
CCTTGATGTTCTGGTTCGAGATCTGCACCTCGGACATCAGCGCGT
CGAGCTGCGCATCGCCGAACACGGTCCACCAGTCGGCGCGTGCCA
GCGCATCCTGCGGCTCGGCGGGCTTCCAGTCGCCGGTCCAGGCGG
GGGTGGCGGCATCGGCTTCCTTGAAGGATGCGGAAACCGGCGCGT
CGGGGCGCTGGTAGTCGGGGCCGACGGCGCAGCCGGCCAGCAGCA
GCGCGCAGGCCAGCGACACCGGCAGGGCA TGGGTCAGGAGGCGG
GAAAGAACTGTCATGTCGAGTCTTCGCAAAT CTAGACGGCGGCC
GGCTGGTCAGGCGTGCCGGCACCACGGCGGCGCTGGCGCCAGGCC
TTGACCTTCAGGCGCCAGCGGTCCAGCGTCAGGTAGACCACCGGC
GTGGTGTACAGCGTCAGCAGCTGGCTTACCACCAGTCCGCCGACA
ATGGAGATGCCCAGCGGCGCGCGCAGTTCGGCGCCGTCGCCGCGG
CCGATTGCCAGCGGCACCGCGCCCAGCAGCGCGGCCATGGTGGTC
ATCAGGATCGGGCGGAAGCGCAGCAGGCAGGCGCGGTAGATCGCG
TCGCGCGGCGACAGGCCATCGCGCCGTTCGGCATCGATGGCGAAG
TCGATCATCATGATCGCGTTCTTTTTCACGATGCCGATCAGCAGG
ATCACGCCGATCAGCGCGATGATGCTGAAGTCGGTCTTCGATGCC
AGCAGCGCCAGCAGCGCGCCCACGCCGGCGGAGGGCAGCGTCGAC
AGGATCGTCAGCGGATGCACATAGCTTTCATACAGCACGCCCAGC
ACGATGTAGATCGTGATCAGCGCCGCCAGGATCAGGATCGGCTGA
CTCTTGAGCGAATCCTGGAACGCCTTGGCGCCGCCCTGGAAGTTG
GCGCGCAGCGTCTCCGGCACGCCGATGCGCGCCATCTCGCGCGTG
ATCGCGTCGGTCGCCTGCGACAGCGAAGTGCCCTCGGCCAGGTTG
AACGAGATCGTCGAGGCCGCGAACTGGCCCTGGTGGTTCACGCCC
AGCGGCGTGCTGGACGGGGTCACGCGCGCGAACGCCGCCAGCGGC
ACGCGGTTGCCGTTGCCGGTGACCACGTAGATGTCCTTGAGCGCA
TCGGGCCCTTGCAGGTATTCCTGGCTCAGCTCCATCACCACGCGG
TACTGGTTCAGCGGATGGTAGATGGTGGACACCAGCCGCTGGCCG
AAGGCATCGTTGAGCACCGCATCCACCTGCTGCGCGGTCACGCCC
AGGCGCGAGGCCGCGTCGCGGTCGATGATCACCGAGGTCTGCAGG
CCCTTGTCGTTGGTATCGGTGTCGATATCCTCCAGCCCCTTCAGG
TTCGACAACGCGGCGCGCACCTTGGGCTCCCACGCGCGCAGCACT
TCCAGGTCGTCC

The third noted mutation may correspond to the sequence listed below ranging from position 2563181-2563281 for Ralstonia eutropha H16 chromosome 1. The bolded, double underlined text indicates a mutation (e.g., nt 101 of SEQ ID NO: 5).

(201 nt)
SEQ ID NO: 5
GCAGCTTGATGCCATTGACGAGGTAGATGGAAACCGGCACGTGCT
CTTTGCGCAGCGCGTTCAGGAACGGGCCTTGTAGCAGTTGCCCTT
TGTTGC TCAT GGCACACTCCAAATTTATAGGTTTAGTGGTGAA
TGATGGGGATGGAAATCCCCGGTTCAAGTCAGGCGGCGCAAAAAC
GCGCCAGAAAAAAGATCA AAAAC

The fourth noted mutation may correspond to the sequence listed below ranging from position 241880-242243 for Ralstonia eutropha H16 chromosome 1. The bolded, double underlined text indicates a mutation (e.g., nt 364-379 of SEQ ID NO: 6).

(479 nt)
SEQ ID NO: 6
GAGGATGCCATGTCCGAAGCGCCTGTCCTTGCCCCCTCGACCTCA
ACCCAGCCGCCCGCCGCCGGCCAGCTCAACCTGATCCGCCCGCAG
CCATATGCCGACTGGGCGCCGCAGGTCACGGCCGAAGAACGCGCC
ACGCTGCGCCGCGAGCTGGAGCAGGGCGCCGTGCTGTACTTCCCG
AACCTGAATTTCCGCTTCCAGCCGGGCGAAGAGCGCTTCCTTGAC
AGCCGCTATTCCGACGGCAAGTCCAAGAACATCAACCTGCGCGCC
GACGACACCGCGGTGCGCGGCGCCCAGGGCAGTCCGCAGGACCTG
GCGGACCTGTACACGCTGATCCGCCGCTACGCCGACAACAGCGAA
TTG CTGGTGCGCACGCTGT TCCCTGAATACATCCCGCACATGA
CGCGCGCCGGCACCTCGCTGCGGCCCAGCGAGATCGCCGGGCGCC
CGGTCAGCTGGCGCAAGGACGACACCCGCCT

In the above sequences, it should be understood that a bacterium may include changes in one or more base pairs relative to the mutation sequences noted above that still produce the same functionality and/or amino acid within the bacteria. For example, a bacterium may include 95%, 96%, 97%, 98%, 99%, or any other appropriate percentage of the same mutation sequences listed above while still providing the noted enhanced ROS resistance.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

In microbiology, “16S sequencing” or “16S rRNA” or “16S-rRNA” or “16S” refers to sequence derived by characterizing the nucleotides that comprise the 16S ribosomal RNA gene(s). The bacterial 16S rDNA is approximately 1500 nucleotides in length and is used in reconstructing the evolutionary relationships and sequence similarity of one bacterial isolate to a second isolate using phylogenetic approaches. 16S sequences are used for phylogenetic reconstruction as they are in general highly conserved, but contain specific hypervariable regions that harbor sufficient nucleotide diversity to differentiate genera and species of most bacteria, as well as fungi.

The “V1-V9 regions” of the 16S rRNA refers to the first through ninth hypervariable regions of the 16S rRNA gene that are used for genetic typing of bacterial samples. These regions in bacteria are defined by nucleotides 69-99, 137-242, 433-497, 576-682, 822-879, 986-1043, 1117-1173, 1243-1294 and 1435-1465 respectively using numbering based on the E, coli system of nomenclature. Brosius et al., Complete nucleotide sequence of a 16S ribosomal RNA gene from Escherichia coli, PNAS 75 (10); 4801-4805 (1978). In some embodiments, at least one of the V1, V2, V3, V4, V5, V6, V7, V8, and V9 regions are used to characterize an OTU. In one embodiment, the V1, V2, and V3 regions are used to characterize an OTU. In another embodiment, the V3, V4, and V5 regions are used to characterize an OTU. In another embodiment, the V4 region is used to characterize an OTU. A person of ordinary skill in the art can identify the specific hypervariable regions of a candidate 16S rRNA by comparing the candidate sequence in question to the reference sequence and identifying the hypervariable regions based on similarity to the reference hypervariable regions.

“Operational taxonomic unit (OTU, plural OTUs)” refers to a terminal leaf in a phylogenetic tree and is defined by a specific genetic sequence and all sequences that share a specified degree of sequence identity to this sequence at the level of species. A “type” or a plurality of “types” of bacteria includes an OTU or a plurality of different OTUs, and also encompasses a strain, species, genus, family or order of bacteria. The specific genetic sequence may be the 16S rRNA sequence or a portion of the 16S rRNA sequence, or it may be a functionally conserved housekeeping gene found broadly across the eubacterial kingdom. OTUs generally share at least 95%, 96%, 97%, 98%, or 99% sequence identity. OTUs are frequently defined by comparing sequences between organisms. Sequences with less than the specified sequence identity (e.g., less than 97%) are not considered to form part of the same OTU.

“Clade” refers to the set of OTUs or members of a phylogenetic tree downstream of a statistically valid node in a phylogenetic tree. The clade comprises a set of terminal leaves in the phylogenetic tree that is a distinct monophyletic evolutionary unit.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein. Preferably, the subject is a mammal. The mammal can be a human, non-human primate, mouse, rat, dog, cat, horse, or cow, but is not limited to these examples. A subject can be male or female. In some embodiments, the subject is a plant. In some embodiments, the subject is a bacterium.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile. Val. Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. activity and specificity of a native or reference polypeptide is retained.

Amino acids can be grouped according to similarities in the properties of their side chains (in A. L. Lehninger, in Biochemistry, second ed., pp. 73-75. Worth Publishers, New York (1975)); (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine. Met. Ala. Val. Leu. Ile; (2) neutral hydrophilic: Cys, Ser, Thr, Asn, Gln; (3) acidic: Asp. Glu; (4) basic: His, Lys, Arg, (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide (or a nucleic acid encoding such a polypeptide) can be a functional fragment of one of the amino acid sequences described herein. As used herein, a “functional fragment” is a fragment or segment of a peptide which retains at least 50% of the wild-type reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant.” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can beat least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73. 1985); Craik (BioTechniques. January 1985, 12-19); Smith et al. (Genetic Engineering: Principles and Methods. Plenum Press. 1981); and U.S. Pat. Nos. 4.518.584 and 4.737.462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell is typically still referred to as “engineered” even though the actual manipulation was performed on a prior entity.

In some embodiments, a nucleic acid encoding a polypeptide as described herein is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E, coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection: an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy. 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers. Inc., 1995 (ISBN 1-56081-569-8); Immunology by Werner Luttmann, published by Elsevier. 2006; Janeway's Immunobiology. Kenneth Murphy, Allan Mowat, Casey Weaver (eds.), W. W. Norton & Company, 2016 (ISBN 0815345054, 978-0815345053); Lewin's Genes X I, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeck, David H Margulies, Ethan M Shevach, Warren Strobe, (eds.) John Wiley and Sons, Inc., 2003 (ISBN 0471142735, 9780471142737), International Patent Publications WO2021158657A1, PCT/US2021/016406, PCT/US2022/015793; the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:

1. A system for producing a bioproduct comprising:
at least one reactor chamber containing therein at least one solution selected from:
a) at least one growth solution comprising:
   i) carbon dioxide (CO₂), hydrogen (H2), and oxygen (O₂); or
   ii) an organic carbon source, hydrogen (H2), and oxygen (O₂), and optionally carbon dioxide (CO₂); and/or
b) at least one production solution comprising:
   i) carbon dioxide (CO₂), hydrogen (H2), and oxygen (O₂);
   ii) an organic carbon source, hydrogen (H2), and oxygen (O₂), and optionally carbon dioxide (CO₂); or
   iii) an organic carbon source and oxygen (O₂); and
wherein the at least one reactor chamber contains therein:
c) at least one microorganism (e.g., bacterium) in the at least one growth solution and/or at least one production solution, wherein the at least one microorganism (e.g., bacterium) produces the bioproduct.
   2. The system of paragraph 1, wherein the system comprises one reactor chamber.
   3. The system of paragraph 1 or 2, wherein at least a portion of the growth solution can be removed from the at least one reactor chamber.
   4. The system of any one of paragraphs 1-3, wherein at least a portion of the production solution can be added to the at least one reactor chamber.
   5. The system of any one of paragraphs 1-4, wherein the system comprises at least two reactor chambers.
   6. The system of any one of paragraphs 1-5, wherein the at least one reactor chamber containing the at least one growth solution is a continuous fermentation reactor chamber.
   7. The system of any one of paragraphs 1-6, wherein the at least one reactor chamber containing the at least one growth solution is a gas fermentation reactor chamber.
   8. The system of any one of paragraphs 1-7, wherein the at least one reactor chamber containing the at least one growth solution is a mixotrophic fermentation reactor chamber.
   9. The system of any one of paragraphs 1-8, wherein the at least one reactor chamber containing the at least one production solution is a fed-batch fermentation reactor chamber.
   10. The system of any one of paragraphs 1-9, wherein the at least one reactor chamber containing the least one production solution is:
a) a gas fermentation reactor chamber;
b) a gas and organic carbon (mixotrophic) fermentation reactor chamber; or
c) an organic carbon fermentation reactor chamber.
   11. The system of any one of paragraphs 1-10, wherein the at least one reactor chamber containing the at least one production solution emits no CO₂.
   12. The system of any one of paragraphs 1-11, wherein the at least one reactor chamber containing the at least one production solution emits no CO₂.
   13. The system of any one of paragraphs 1-12, wherein the at least one reactor chamber containing the at least one production solution emits at most 1 molecule of CO₂ per molecule of acetyl-CoA.
   14. The system of any one of paragraphs 1-13, wherein the at least one reactor chamber further comprises a pair of electrodes in contact with the first and/or at least one production solution that split water to form the hydrogen.
   15. The system of any one of paragraphs 1-14, wherein the at least one reactor chamber further comprises an isolated gas volume above a surface of the first and/or at least one production solution within a headspace of the at least one reactor chamber.
   16. The system of paragraph 15, wherein the isolated gas volume comprises carbon dioxide (CO₂), hydrogen (H2), and/or oxygen (O₂).
   17. The system of any one of paragraphs 1-16, wherein the at least one reactor chamber further comprises a power source comprising a renewable source of energy.
   18. The system of paragraph 17, wherein the renewable source of energy comprises a solar cell, wind turbine, generator, battery, or grid power.
   19. A system for producing a bioproduct comprising:
a) a primary reactor chamber with a at least one growth solution contained therein,
wherein the at least one growth solution comprises:
   i) carbon dioxide (CO₂), hydrogen (H2), and oxygen (O₂), or
   ii) an organic carbon source, hydrogen (H2), and oxygen (O₂), and optionally carbon dioxide (CO₂);
b) at least one secondary reactor chamber with a at least one production solution contained therein, wherein the at least one production solution comprises:
   i) carbon dioxide (CO₂), hydrogen (H2), and oxygen (O₂);
   ii) an organic carbon source, hydrogen (H2), and oxygen (O₂), and optionally carbon dioxide (CO₂); or
   iii) an organic carbon source and oxygen (O₂); and
c) at least one microorganism (e.g., bacterium) in the at least one growth solution of the primary reactor chamber and/or at least one production solution of the secondary reactor chamber, wherein the at least one microorganism (e.g., bacterium) produces the bioproduct.
   20. The system of paragraph 19, wherein the system comprises at least two secondary reactor chambers.
   21. The system of paragraph 19 or 20, wherein the system comprises three secondary reactor chambers, wherein:
a) the solution in the first secondary reactor chamber comprises carbon dioxide (CO₂), hydrogen (H2), and oxygen (O₂);
b) the solution in the second secondary reactor chamber comprises an organic carbon source, hydrogen (H2), and oxygen (O₂); and
c) the solution in the third secondary reactor chamber comprises an organic carbon source and oxygen (O₂).
   22. The system of any one of paragraphs 19-21, wherein the primary reactor chamber is a continuous fermentation reactor chamber.
   23. The system of any one of paragraphs 19-22, wherein the primary reactor chamber is a gas fermentation reactor chamber.
   24. The system of any one of paragraphs 19-23, wherein the primary reactor chamber is a mixotrophic fermentation reactor chamber.
   25. The system of any one of paragraphs 19-24, wherein the secondary reactor chamber is a fed-batch fermentation reactor chamber.
   26. The system of any one of paragraphs 19-25, wherein the primary and at least one secondary reactor chambers are physically linked.
   27. The system of any one of paragraphs 19-26, wherein the at least one growth solution from the primary reactor chamber is batch fed into the secondary reactor chamber.
   28. The system of any one of paragraphs 19-27, wherein the secondary reactor chamber is:
a) a gas fermentation reactor chamber;
b) a gas and organic carbon (mixotrophic) fermentation reactor chamber; or
c) an organic carbon fermentation reactor chamber.
   29. The system of any one of paragraphs 19-28, wherein the primary reactor chamber emits no CO₂.
   30. The system of any one of paragraphs 19-29, wherein the secondary reactor chamber emits no CO₂.
   31. The system of any one of paragraphs 19-30, wherein the secondary reactor chamber emits at most 1 molecule of CO₂ per molecule of acetyl-CoA.
   32. The system of any one of paragraphs 19-31, wherein the primary and/or secondary reactor chamber further comprises a pair of electrodes in contact with the first and/or at least one production solution that split water to form the hydrogen.
   33. The system of any one of paragraphs 19-32, wherein the primary and/or secondary reactor chamber further comprises an isolated gas volume above a surface of the at least one growth solution and/or at least one production solution within a headspace of the primary and/or secondary reactor chamber.
   34. The system of paragraph 33, wherein the isolated gas volume comprises carbon dioxide (CO₂), hydrogen (H2), and/or oxygen (O₂).
   35. The system of any one of paragraphs 19-34, wherein the primary and/or secondary reactor chamber further comprises a power source comprising a renewable source of energy.
   36. The system of paragraph 35, wherein the renewable source of energy comprises a solar cell, wind turbine, generator, battery, or grid power.
   37. The system of any one of paragraphs 1-36, wherein the system comprises:
a) one growth solution and one production solution:
b) two growth solutions and one production solution; or
c) one growth solution and two production solutions.
   38. The system of any one of paragraphs 1-37, wherein the system further comprises at least one inducer solution.
   39. The system of any one of paragraphs 1-38, wherein the at least one inducer solution comprises a level of bioavailable nitrogen below a pre-determined threshold.
   40. The system of any one of paragraphs 1-39, wherein the at least one inducer solution comprises arabinose.
   41. The system of any one of paragraphs 1-40, wherein the at least one inducer solution further comprises:
a) carbon dioxide (CO₂), hydrogen (H2), and oxygen (O₂);
b) an organic carbon source, hydrogen (H2), and oxygen (O₂), and optionally carbon dioxide (CO₂); or
c) an organic carbon source and oxygen (O₂).
   42. The system of any one of paragraphs 1-41, wherein the microorganism (e.g., bacterium) is a chemolithotroph.
   43. The system of any one of paragraphs 1-42, wherein the microorganism (e.g., bacterium) is a mixotroph.
   44. The system of any one of paragraphs 1-43, wherein the mixotroph is capable of gas fermentation and organic carbon fermentation.
   45. The system of any one of paragraphs 1-44, wherein the microorganism (e.g., bacterium) is a switchotroph.
   46. The system of any one of paragraphs 1-45, wherein the switchotroph is capable of switching between gas fermentation and organic carbon fermentation.
   47. The system of any one of paragraphs 1-46, wherein the microorganism (e.g., bacterium) is not a heterotroph.
   48. The system of any one of paragraphs 1-47, wherein the microorganism (e.g., bacterium) is Cupriavidus necator.
   49. The system of any one of paragraphs 1-48, wherein the microorganism (e.g., bacterium) naturally produces the bioproduct.
   50. The system of any one of paragraphs 1-49, wherein the microorganism (e.g., bacterium) is engineered to produce the bioproduct.
   51. The system of any one of paragraphs 1-50, wherein the microorganism (e.g., bacterium) is capable of being induced to produce the bioproduct.
   52. The system of any one of paragraphs 1-51, wherein the bioproduct is capable of being isolated, collected, or concentrated after the microorganism (e.g., bacterium) produces a pre-determined concentration of the bioproduct.
   53. The system of any one of paragraphs 1-52, wherein the organic carbon source is selected from the group consisting of: glucose, glycerol, gluconate, acetate, fructose, decanoate, fatty acid, and glycerol gluconate.
   54. The system of any one of paragraphs 1-53, wherein the organic carbon source comprises glucose.
   55. The system of any one of paragraphs 1-54, wherein the at least one growth solution and/or at least one production solution comprises cell culture medium.
   56. The system of any one of paragraphs 1-55, wherein the at least one growth solution and/or at least one production solution comprises defined medium.
   57. The system of any one of paragraphs 1-56, wherein the at least one growth solution and/or at least one production solution comprises minimal medium.
   58. The system of any one of paragraphs 1-57, wherein the at least one growth solution and/or at least one production solution comprises rich medium.
   59. The system of any one of paragraphs 1-58, wherein the bioproduct is selected from the group consisting of: polypeptide, glycoprotein, lipoprotein, lipid, monosaccharide, polysaccharide, nucleic acid, small molecule, or metabolite.
   60. The system of any one of paragraphs 1-59, wherein the bioproduct is selected from the group consisting of: polyhydroxyalkanoate (PHA); sucrose; lipochitooligosaccharide; and triacylglyceride.
   61. A method of a culturing a microorganism (e.g., bacterium), the method comprising:
a) culturing the microorganism (e.g., bacterium) in at least one reactor chamber with at least one growth solution contained therein, wherein the at least one growth solution comprises:
   i) carbon dioxide (CO₂), hydrogen (H2), and oxygen (O₂); or
   ii) an organic carbon source, hydrogen (H2), and oxygen (O₂), and optionally carbon dioxide (CO₂);
b) adding at least one production solution to the at least one reactor chamber, wherein the at least one production solution comprises:
   i) carbon dioxide (CO₂), hydrogen (H2), and oxygen (O₂);
   ii) an organic carbon source, hydrogen (H2), and oxygen (O₂), and optionally carbon dioxide (CO₂); or
   iii) an organic carbon source and oxygen (O₂); and
c) culturing the microorganism (e.g., bacterium) in the at least one production solution.
   62. A method of a culturing a microorganism (e.g., bacterium), comprising:
a) culturing the microorganism (e.g., bacterium) in at least one reactor chamber with at least one growth solution contained therein, wherein the at least one growth solution comprises:
   i) carbon dioxide (CO₂), hydrogen (H2), and oxygen (O₂); or
   ii) an organic carbon source, hydrogen (H2), and oxygen (O₂), and optionally carbon dioxide (CO₂);
b) adding at least one production solution to the at least one reactor chamber, wherein the at least one production solution comprises:
   i) carbon dioxide (CO₂), hydrogen (H2), and oxygen (O₂);
   ii) an organic carbon source, hydrogen (H2), and oxygen (O₂), and optionally carbon dioxide (CO₂); or
   iii) an organic carbon source and oxygen (O₂);
c) culturing the microorganism (e.g., bacterium) in the at least one production solution; and
d) isolating, collecting, or concentrating the bioproduct from the microorganism (e.g., bacterium) in the at least one reactor chamber or from the at least one production solution in the at least one reactor chamber.
   63. The method of paragraph 61 or 62, wherein the microorganism (e.g., bacterium) is cultured in the at least one growth solution for a sufficient amount of time and under sufficient conditions for the microorganism (e.g., bacterium) to grow to a pre-determined concentration.
   64. The method of any one of paragraphs 61-63, wherein the microorganism (e.g., bacterium) does not produce the bioproduct in the at least one growth solution.
   65. The method of any one of paragraphs 61-64, wherein at least a portion of the at least one growth solution is removed from the at least one reactor chamber after the microorganism (e.g., bacterium) grows to a pre-determined concentration.
   66. The method of any one of paragraphs 61-65, wherein at least a portion of the at least one production solution is added to the at least one reactor chamber after the microorganism (e.g., bacterium) grows to a pre-determined concentration.
   67. The method of any one of paragraphs 61-66, wherein at least a portion of the at least one growth solution is removed from the at least one reactor chamber whenever the microorganism (e.g., bacterium) grows to a pre-determined concentration such that the microorganism (e.g., bacterium) does not ever exceed the pre-determined concentration.
   68. The method of any one of paragraphs 61-67, wherein at least a portion of the at least one production solution is added to the at least one reactor chamber whenever the microorganism (e.g., bacterium) grows to a pre-determined concentration such that the microorganism (e.g., bacterium) does not ever exceed the pre-determined concentration.
   69. The method of any one of paragraphs 61-68, wherein the microorganism (e.g., bacterium) is cultured in the at least one production solution for a sufficient amount of time and under sufficient conditions for the microorganism (e.g., bacterium) to produce a pre-determined concentration of the bioproduct.
   70. The method of any one of paragraphs 61-69, wherein the microorganism (e.g., bacterium) does not exhibit substantial growth in the at least one production solution.
   71. The method of any one of paragraphs 61-70, wherein the at least one reactor chamber containing the at least one growth solution is a continuous fermentation reactor chamber.
   72. The method of any one of paragraphs 61-71, wherein the at least one reactor chamber containing the at least one growth solution is a gas fermentation reactor chamber.
   73. The method of any one of paragraphs 61-72, wherein the at least one reactor chamber containing the at least one growth solution is a mixotrophic fermentation reactor chamber.
   74. The method of any one of paragraphs 61-73, wherein the at least one reactor chamber containing the at least one production solution is a fed-batch fermentation reactor chamber.
   75. The method of any one of paragraphs 61-74, wherein the at least one reactor chamber containing the at least one production solution is:
a) a gas fermentation reactor chamber;
b) a gas and organic carbon (mixotrophic) fermentation reactor chamber; or
c) an organic carbon fermentation reactor chamber.
   76. The method of any one of paragraphs 61-75, wherein the at least one reactor chamber containing the at least one growth solution emits no CO₂.
   77. The method of any one of paragraphs 61-76, wherein the at least one reactor chamber containing the at least one production solution emits no CO₂.
   78. The method of any one of paragraphs 61-77, wherein the at least one reactor chamber containing the at least one production solution emits at most 1 molecule of CO₂ per molecule of acetyl-CoA.
   79. The method of any one of paragraphs 61-78, wherein the at least one reactor chamber further comprises a pair of electrodes in contact with the first and/or at least one production solution that split water to form the hydrogen.
   80. The method of any one of paragraphs 61-79, wherein the at least one reactor chamber further comprises an isolated gas volume above a surface of the first and/or at least one production solution within a headspace of the at least one reactor chamber.
   81. The method of paragraph 80, wherein the isolated gas volume comprises carbon dioxide (CO₂), hydrogen (H2), and/or oxygen (O₂).
   82. The method of any one of paragraphs 61-81, wherein the at least one reactor chamber further comprises a power source comprising a renewable source of energy.
   83. The method of paragraph 82, wherein the renewable source of energy comprises a solar cell, wind turbine, generator, battery, or grid power.
   84. A method of a culturing a microorganism (e.g., bacterium), the method comprising:
a) culturing the microorganism (e.g., bacterium) in a primary reactor chamber with a at least one growth solution contained therein, wherein the at least one growth solution comprises:
   i) carbon dioxide (CO₂), hydrogen (H2), and oxygen (O₂); or
   ii) an organic carbon source, hydrogen (H2), and oxygen (O₂), and optionally carbon dioxide (CO₂);
b) moving at least a portion of the at least one growth solution from the primary reactor chamber into at least one secondary reactor chamber with a at least one production solution contained therein, wherein the at least one production solution comprises:
   i) carbon dioxide (CO₂), hydrogen (H2), and oxygen (O₂);
   ii) an organic carbon source, hydrogen (H2), and oxygen (O₂), and optionally carbon dioxide (CO₂); or
   iii) an organic carbon source and oxygen (O₂); and
c) culturing the microorganism (e.g., bacterium) in the secondary reactor chamber.
   85. A method of producing a bioproduct, comprising:
a) culturing a microorganism (e.g., bacterium) that produces a bioproduct in a primary reactor chamber with a at least one growth solution contained therein, wherein the at least one growth solution comprises:
   i) carbon dioxide (CO₂), hydrogen (H2), and oxygen (O₂); or
   ii) an organic carbon source, hydrogen (H2), and oxygen (O₂), and optionally carbon dioxide (CO₂);
b) moving at least a portion of the at least one growth solution from the primary reactor chamber into a secondary reactor chamber with a at least one production solution contained therein, wherein the at least one production solution comprises:
   i) carbon dioxide (CO₂), hydrogen (H2), and oxygen (O₂);
   ii) an organic carbon source, hydrogen (H2), and oxygen (O₂), and optionally carbon dioxide (CO₂); or
   iii) an organic carbon source and oxygen (O₂);
c) culturing the microorganism (e.g., bacterium) in the secondary reactor chamber; and
d) isolating, collecting, or concentrating the bioproduct from the microorganism (e.g., bacterium) in the secondary reactor chamber or from the at least one production solution in the second reactor chamber.
   86. The method of paragraphs 84 or 85, wherein the microorganism (e.g., bacterium) is cultured in the primary reactor chamber for a sufficient amount of time and under sufficient conditions for the microorganism (e.g., bacterium) to grow to a pre-determined concentration.
   87. The method of any one of paragraphs 84-86, wherein the microorganism (e.g., bacterium) does not produce the bioproduct in the primary reactor chamber.
   88. The method of any one of paragraphs 84-87, wherein at least a portion of the at least one growth solution from the primary reactor chamber is moved into the at least one secondary reactor chamber after the microorganism (e.g., bacterium) grows to a pre-determined concentration.
   89. The method of any one of paragraphs 84-88, wherein the method comprises the following iterative steps:
a) moving at least a portion of the at least one growth solution from the primary reactor chamber into a first secondary reactor chamber after the microorganism (e.g., bacterium) grows to a pre-determined concentration; and
b) moving at least a portion of the at least one growth solution from the primary reactor chamber into a second secondary reactor chamber after the microorganism (e.g., bacterium) grows to a pre-determined concentration.
   90. The method of any one of paragraphs 84-89, wherein the method comprises the following iterative steps:
a) moving at least a portion of the at least one growth solution from the primary reactor chamber into a first secondary reactor chamber after the microorganism (e.g., bacterium) grows to a pre-determined concentration:
b) moving at least a portion of the at least one growth solution from the primary reactor chamber into a second secondary reactor chamber after the microorganism (e.g., bacterium) grows to a pre-determined concentration; and
c) moving at least a portion of the at least one growth solution from the primary reactor chamber into a third secondary reactor chamber after the microorganism (e.g., bacterium) grows to a pre-determined concentration.
   91. The method of any one of paragraphs 84-90, wherein a portion of the at least one growth solution from the primary reactor chamber is moved into at least one secondary reactor chamber whenever the microorganism (e.g., bacterium) grows to a pre-determined concentration such that the microorganism (e.g., bacterium) does not ever exceed the pre-determined concentration.
   92. The method of any one of paragraphs 84-91, wherein the microorganism (e.g., bacterium) is cultured in the secondary reactor chamber for a sufficient amount of time and under sufficient conditions for the microorganism (e.g., bacterium) to produce a pre-determined concentration of the bioproduct.
   93. The method of any one of paragraphs 84-92, wherein the microorganism (e.g., bacterium) does not exhibit substantial growth in the at least one secondary reactor chamber.
   94. The method of any one of paragraphs 84-93, wherein the primary reactor chamber is a continuous fermentation reactor chamber.
   95. The method of any one of paragraphs 84-94, wherein the primary reactor chamber is a gas fermentation reactor chamber.
   96. The method of any one of paragraphs 84-95, wherein the primary reactor chamber is a mixotrophic fermentation reactor chamber.
   97. The method of any one of paragraphs 84-96, wherein the secondary reactor chamber is a fed-batch fermentation reactor chamber.
   98. The method of any one of paragraphs 84-97, wherein the primary and at least one secondary reactor chambers are physically linked.
   99. The method of any one of paragraphs 84-98, wherein the at least one growth solution from the primary reactor chamber is batch fed into the secondary reactor chamber.
   100. The method of any one of paragraphs 84-99, wherein the secondary reactor chamber is:
a) a gas fermentation reactor chamber;
b) a gas and organic carbon (mixotrophic) fermentation reactor chamber; or
c) an organic carbon fermentation reactor chamber.
   101. The method of any one of paragraphs 84-100, wherein the primary reactor chamber emits no CO₂.
   102. The method of any one of paragraphs 84-101, wherein the secondary reactor chamber emits no CO₂.
   103. The method of any one of paragraphs 84-102, wherein the secondary reactor chamber emits at most 1 molecule of CO₂ per molecule of acetyl-CoA.
   104. The method of any one of paragraphs 84-103, wherein the primary and/or secondary reactor chamber further comprises a pair of electrodes in contact with the at least one growth solution and/or at least one production solution that split water to form the hydrogen.
   105. The method of any one of paragraphs 84-104, wherein the primary and/or secondary reactor chamber further comprises an isolated gas volume above a surface of the at least one growth solution and/or at least one production solution within a headspace of the primary and/or secondary reactor chamber.
   106. The method of paragraph 105, wherein the isolated gas volume comprises carbon dioxide (CO₂), hydrogen (H2), and/or oxygen (O₂).
   107. The method of any one of paragraphs 84-106, wherein the primary and/or secondary reactor chamber further comprises a power source comprising a renewable source of energy.
   108. The method of paragraph 107, wherein the renewable source of energy comprises a solar cell, wind turbine, generator, battery, or grid power.
   109. The method of any one of paragraphs 61-108, wherein the method comprises using:
a) one growth solution and one production solution:
b) two growth solutions and one production solution; or
c) one growth solution and two production solutions.
   110. The method of any one paragraphs 61-109, wherein the method further comprises adding at least one inducer solution to the at least one reactor chamber;
   111. The method of any one paragraphs 61-110, wherein the at least one inducer solution is added after the microorganism (e.g., bacterium) is cultured in the at least one growth solution for a sufficient amount of time and under sufficient conditions for the microorganism (e.g., bacterium) to grow to a pre-determined concentration.
   112. The method of any one paragraphs 61-111, wherein the inducer solution induces the microorganism (e.g., bacterium) to produce the bioproduct.
   113. The method of any one of paragraphs 61-112, wherein the at least one inducer solution comprises a level of bioavailable nitrogen below a pre-determined threshold.
   114. The method of any one of paragraphs 61-113, wherein the at least one inducer solution comprises arabinose.
   115. The method of any one of paragraphs 61-114, wherein the at least one inducer solution further comprises:
a) carbon dioxide (CO₂), hydrogen (H2), and oxygen (O₂);
b) an organic carbon source, hydrogen (H2), and oxygen (O₂), and optionally carbon dioxide (CO₂); or
c) an organic carbon source and oxygen (O₂).
   116 The method of any one of paragraphs 61-115, wherein the microorganism (e.g., bacterium) is a chemolithotroph.
   117. The method of any one of paragraphs 61-116, wherein the microorganism (e.g., bacterium) is a mixotroph.
   118. The method of any one of paragraphs 61-117, wherein the mixotroph is capable of gas fermentation and organic carbon fermentation.
   119. The method of any one of paragraphs 61-118, wherein the microorganism (e.g., bacterium) is a switchotroph.
   120. The method of any one of paragraphs 61-119, wherein the switchotroph is capable of switching between gas fermentation and organic carbon fermentation.
   121. The method of any one of paragraphs 61-120, wherein the microorganism (e.g., bacterium) is not a heterotroph.
   122. The method of any one of paragraphs 61-121, wherein the microorganism (e.g., bacterium) is Cupriavidus necator.
   123. The method of any one of paragraphs 61-122, wherein the microorganism (e.g., bacterium) naturally produces the bioproduct.
   124. The method of any one of paragraphs 61-123, wherein the microorganism (e.g., bacterium) is engineered to produce the bioproduct.
   125. The method of any one of paragraphs 61-124, wherein the bioproduct is isolated, collected, or concentrated after the microorganism (e.g., bacterium) produces a pre-determined concentration of the bioproduct.
   126. The method of any one of paragraphs 61-125, wherein the organic carbon source is selected from the group consisting of: glucose, glycerol, gluconate, acetate, fructose, decanoate, fatty acid, and glycerol gluconate.
   127. The method of any one of paragraphs 61-126, wherein the organic carbon source comprises glucose.
   128. The method of any one of paragraphs 61-127, wherein the at least one growth solution and/or at least one production solution comprises cell culture medium.
   129. The method of any one of paragraphs 61-128, wherein the at least one growth solution and/or at least one production solution comprises defined medium.
   130. The method of any one of paragraphs 61-129, wherein the at least one growth solution and/or at least one production solution comprises minimal medium.
   131. The method of any one of paragraphs 61-130, wherein the at least one growth solution and/or at least one production solution comprises rich medium.
   132. The method of any one of paragraphs 61-131, wherein the bioproduct is selected from the group consisting of: polypeptide, glycoprotein, lipoprotein, lipid, monosaccharide, polysaccharide, nucleic acid, small molecule, or metabolite.
   133. The method of any one of paragraphs 61-132, wherein the bioproduct is selected from the group consisting of: polyhydroxyalkanoate (PHA); sucrose: lipochitooligosaccharide; and triacylglyceride.
   134. A method of adapting the metabolism of a microorganism (e.g., bacterium) for gas fermentation, the method comprising:
a) culturing the microorganism (e.g., bacterium) in a solution comprising an organic carbon source; and
b) transitioning the microorganism (e.g., bacterium) to a gas fermentation solution lacking an organic carbon source once the microorganism (e.g., bacterium) grows to a pre-determined concentration.
   135. The method of paragraph 134, wherein the organic carbon source is selected from the group consisting of: glucose, glycerol, gluconate, acetate, fructose, decanoate, fatty acid, and glycerol gluconate.

Some embodiments of the technology described herein can be defined according to any of the following numbered paragraphs:

1. A system for producing a bioproduct comprising:
a) a primary reactor chamber with a first solution contained therein, wherein the first solution comprises carbon dioxide (CO₂), hydrogen (H2), and oxygen (O₂);
b) at least one secondary reactor chamber with a second solution contained therein, wherein the second solution comprises:
   i) carbon dioxide (CO₂), hydrogen (H2), and oxygen (O₂);
   ii) an organic carbon source, hydrogen (H2), and oxygen (O₂); or
   iii) an organic carbon source and oxygen (O₂); and
c) at least one microorganism (e.g., bacterium) in the first solution of the primary reactor chamber and/or second solution of the secondary reactor chamber, wherein the at least one microorganism (e.g., bacterium) produces the bioproduct.
   2. The system of paragraph 1, wherein the system comprises at least two secondary reactor chambers.
   3. The system of paragraph 1 or 2, wherein the system comprises three secondary reactor chambers, wherein:
a) the solution in the first secondary reactor chamber comprises carbon dioxide (CO₂), hydrogen (H2), and oxygen (O₂);
b) the solution in the second secondary reactor chamber comprises an organic carbon source, hydrogen (H2), and oxygen (O₂); and
c) the solution in the third secondary reactor chamber comprises an organic carbon source and oxygen (O₂).
   4. A method of a culturing a microorganism (e.g., bacterium), comprising:
a) culturing the microorganism (e.g., bacterium) in a primary reactor chamber with a first solution contained therein, wherein the first solution comprises carbon dioxide (CO₂), hydrogen (H2), and oxygen (O₂);
b) moving at least a portion of the first solution from the primary reactor chamber into at least one secondary reactor chamber with a second solution contained therein, wherein the second solution comprises:
   i) carbon dioxide (CO₂), hydrogen (H2), and oxygen (O₂);
   ii) an organic carbon source, hydrogen (H2), and oxygen (O₂); or
   iii) an organic carbon source and oxygen (O₂); and
c) culturing the microorganism (e.g., bacterium) in the secondary reactor chamber.
   5. A method of producing a bioproduct, comprising:
a) culturing a microorganism (e.g., bacterium) that produces a bioproduct in a primary reactor chamber with a first solution contained therein, wherein the first solution comprises carbon dioxide (CO₂), hydrogen (H2), and oxygen (O₂);
b) moving at least a portion of the first solution from the primary reactor chamber into a secondary reactor chamber with a second solution contained therein, wherein the second solution comprises:
   i) carbon dioxide (CO₂), hydrogen (H2), and oxygen (O₂);
   ii) an organic carbon source, hydrogen (H2), and oxygen (O₂); or
   iii) an organic carbon source and oxygen (O₂);
c) culturing the microorganism (e.g., bacterium) in the secondary reactor chamber; and
d) isolating, collecting, or concentrating the bioproduct from the microorganism (e.g., bacterium) in the secondary reactor chamber or from the second solution in the second reactor chamber.
   6. The system or method of any one of paragraphs 1-5, wherein the microorganism (e.g., bacterium) is a chemolithotroph.
   7. The system or method of any one of paragraphs 1-6, wherein the microorganism (e.g., bacterium) is a mixotroph.
   8. The system or method of any one of paragraphs 1-7, wherein the mixotroph is capable of gas fermentation and organic carbon fermentation.
   9. The system or method of any one of paragraphs 1-8, wherein the microorganism (e.g., bacterium) is a switchotroph.
   10. The system or method of any one of paragraphs 1-9, wherein the switchotroph is capable of switching between gas fermentation and organic carbon fermentation.
   11. The system or method of any one of paragraphs 1-10, wherein the microorganism (e.g., bacterium) is not a heterotroph.
   12. The system or method of any one of paragraphs 1-11, wherein the microorganism (e.g., bacterium) is Cupriavidus necator.
   13. The system or method of any one of paragraphs 1-12, wherein the microorganism (e.g., bacterium) naturally produces the bioproduct.
   14. The system or method of any one of paragraphs 1-13, wherein the microorganism (e.g., bacterium) is engineered to produce the bioproduct.
   15. The method of any one of paragraphs 4-14, wherein the microorganism (e.g., bacterium) is cultured in the primary reactor chamber for a sufficient amount of time and under sufficient conditions for the microorganism (e.g., bacterium) to grow to a pre-determined concentration.
   16. The method of any one of paragraphs 4-15, wherein the microorganism (e.g., bacterium) does not produce the bioproduct in the primary reactor chamber.
   17. The method of any one of paragraphs 4-16, wherein at least a portion of the first solution from the primary reactor chamber is moved into the at least one secondary reactor chamber after the microorganism (e.g., bacterium) grows to a pre-determined concentration.
   18. The method of any one of paragraphs 4-17, wherein the method comprises the following iterative steps:
a) a portion of the first solution from the primary reactor chamber is moved into a first secondary reactor chamber after the microorganism (e.g., bacterium) grows to a pre-determined concentration; and
b) a portion of the first solution from the primary reactor chamber is moved into a second secondary reactor chamber after the microorganism (e.g., bacterium) grows to a pre-determined concentration.
   19. The method of any one of paragraphs 4-18, wherein the method comprises the following iterative steps:
a) a portion of the first solution from the primary reactor chamber is moved into a first secondary reactor chamber after the microorganism (e.g., bacterium) grows to a pre-determined concentration:
b) a portion of the first solution from the primary reactor chamber is moved into a second secondary reactor chamber after the microorganism (e.g., bacterium) grows to a pre-determined concentration; and
c) a portion of the first solution from the primary reactor chamber is moved into a third secondary reactor chamber after the microorganism (e.g., bacterium) grows to a pre-determined concentration.
   20. The method of paragraph 18 or 19, wherein a portion of the first solution from the primary reactor chamber is moved into at least one secondary reactor chamber whenever the microorganism (e.g., bacterium) grows to a pre-determined concentration such that the microorganism (e.g., bacterium) does not ever exceed the pre-determined concentration.
   21. The method of any one of paragraphs 4-20, wherein the microorganism (e.g., bacterium) is cultured in the secondary reactor chamber for a sufficient amount of time and under sufficient conditions for the microorganism (e.g., bacterium) to produce a pre-determined concentration of the bioproduct.
   22. The method of any one of paragraphs 4-21, further comprising inducing the microorganism (e.g., bacterium) to produce the bioproduct.
   23. The method of any one of paragraphs 4-22, wherein the microorganism (e.g., bacterium) does not exhibit substantial growth in the at least one secondary reactor chamber.
   24. The method of any one of paragraphs 4-23, wherein the bioproduct is isolated, collected, or concentrated after the microorganism (e.g., bacterium) produces a pre-determined concentration of the bioproduct.
   25. The system or method of any one of paragraphs 1-24, wherein the primary reactor chamber is a continuous fermentation reactor chamber.
   26. The system or method of any one of paragraphs 1-25, wherein the primary reactor chamber is a gas fermentation reactor chamber.
   27. The system or method of any one of paragraphs 1-26, wherein the secondary reactor chamber is a fed-batch fermentation reactor chamber.
   28. The system or method of any one of paragraphs 1-27, wherein the primary and at least one secondary reactor chambers are physically linked.
   29. The system or method of any one of paragraphs 1-28, wherein the first solution from the primary reactor chamber is batch fed into the secondary reactor chamber.
   30. The system or method of any one of paragraphs 1-29, wherein the secondary reactor chamber is:
a) a gas fermentation reactor chamber;
b) a gas and organic carbon (mixotrophic) fermentation reactor chamber; or
c) an organic carbon fermentation reactor chamber.
   31. The system or method of any one of paragraphs 1-30, wherein the second solution comprises:
a) at least 11 molecules of H2 per molecule of acetyl-CoA:
b) at least ⅓ molecule of organic carbon source and 5/3 molecules of H2 per molecule of acetyl-CoA; or
c) at least ½ molecule of organic carbon source per molecule of acetyl-CoA.
   32. The system or method of any one of paragraphs 1-31, wherein the second solution comprises:
a) at least 11 molecules of H2 per molecule of acetyl-CoA:
b) at least 1 molecule of organic carbon source and 5 molecules of H2 per 3 molecules of acetyl-CoA; or
c) at least 1 molecule of organic carbon source per 2 molecules of acetyl-CoA.
   33. The system or method of any one of paragraphs 1-32, wherein the organic carbon source is selected from the group consisting of: glucose, glycerol, gluconate, acetate, fructose, decanoate, fatty acid, and glycerol gluconate.
   34. The system or method of any one of paragraphs 1-33, wherein the organic carbon source comprises glucose.
   35. The system or method of any one of paragraphs 1-34, wherein the primary reactor chamber emits no CO₂.
   36. The system or method of any one of paragraphs 1-35, wherein the secondary reactor chamber emits no CO₂.
   37. The system or method of any one of paragraphs 1-36, wherein the secondary reactor chamber emits at most 1 molecule of CO₂ per molecule of acetyl-CoA.
   38. The system or method of any one of paragraphs 1-37, wherein the first and/or second solution comprises cell culture medium.
   39. The system or method of any one of paragraphs 1-38, wherein the first and/or second solution comprises defined medium.
   40. The system or method of any one of paragraphs 1-39, wherein the first and/or second solution comprises minimal medium.
   41. The system or method of any one of paragraphs 1-40, wherein the first and/or second solution comprises rich medium.
   42. The system or method of any one of paragraphs 1-41, wherein the bioproduct is selected from the group consisting of: polypeptide, glycoprotein, lipoprotein, lipid, monosaccharide, polysaccharide, nucleic acid, small molecule, or metabolite.
   43. The system or method of any one of paragraphs 1-42, wherein the bioproduct is selected from the group consisting of: polyhydroxyalkanoate (PHA); sucrose: lipochitooligosaccharide; and triacylglyceride.
   44. The system or method of any one of paragraphs 1-43, wherein the primary and/or secondary reactor chamber further comprises a pair of electrodes in contact with the first and/or second solution that split water to form the hydrogen.
   45. The system or method of any one of paragraphs 1-44, wherein the primary and/or secondary reactor chamber further comprises an isolated gas volume above a surface of the first and/or second solution within a headspace of the primary and/or secondary reactor chamber.
   46. The system or method of paragraph 45, wherein the isolated gas volume comprises carbon dioxide (CO₂), hydrogen (H2), and/or oxygen (O₂).
   47. The system or method of any one of paragraphs 1-46, wherein the primary and/or secondary reactor chamber further comprises a power source comprising a renewable source of energy.
   48. The system or method of paragraph 47, wherein the renewable source of energy comprises a solar cell, wind turbine, generator, battery, or grid power.

The technology described herein is further illustrated by the following examples which in no way should be construed as being further limiting.

### Examples

### Example 1; Carbon Efficient Two-Phase High-Productivity Fermentation System

A sustainable future relies on minimizing the use of petro chemicals and reducing greenhouse gas emissions. Compared to commonly used carbohydrate-based feedstocks, gaseous feedstocks are more cost-effective, are less land-intensive, have fewer restrictions to delivery in large volumes, and have smaller carbon footprints. Synthetic biology tools can be used to genetically engineer Cupriavidus necator and develop efficient mixotrophic and lithotrophic production modes with state of the art fermentation technology.

The overall objective is to demonstrate a carbon-neutral precision fermentation system. The system is a hybrid of continuous gas fermentation (H2/O₂/CO₂) for biomass production and subsequent fed-batch mixotrophic fermentation (sugar and H2). The continuous fermentation provides an austere environment unfavorable to contamination, and because is used solely to produce biomass, it minimizes genetic drift otherwise seen in actively-expressing engineered strains. The second fermentation process includes a sugar feedstock to reach high rates and titers. The second process uses hydrogen to draw down any released CO₂ from growth on sugar, optimizing production and minimizing CO₂ output. Nitrogen limitation and the sugar feedstock is used to induce production of target chemicals. Analogous to the first process, the second fermentation is fed-batch and feeds cells solely for production, not growth, so genetic drift here is also minimized.

Other bioproduction platforms have limitations with regard to carbon efficiency, product versatility and/or productivity. Corn ethanol has high productivity but is carbon inefficient. Acetogenic ethanol production has achieved commercial scale and is a great alternative for low carbon alcohols and acids. Algal biodiesel production was considered a path for higher carbon fuels but has yet to achieve commercial viability.

These platforms have laid a foundation which have aided the development of the next generation of carbon-efficient bioproduction. Efficient established infrastructure can be drawn on for cheap sugar supply, mature gas fermentation process technology, and sophisticated strategies to engineer fatty acid metabolism. These infrastructures can be leveraged to transition to a carbon-efficient, highly productive bioeconomy for energy-rich long-chain carbon molecules with applications in a vast array of industries including fuels, materials and chemicals.

This system seeks to address the limitations of current approaches and incumbent technologies in the manufacturing of biofuels through the application and improvement of process technologies proven in acetogenic production, to aerobic, mixotrophic production with C. necator. Through the use of gas-fermentation technology and synthetic biology, the flexible feedstock platform for mixotrophic production from CO₂. H2, and organic carbon substrates, is capable of producing a wide range of bioproducts. Due to trade-offs between carbon efficiency and productivity that exist within commercialized technologies, the approach described here has not been previously pursued. There exist highly productive bioprocesses that utilize agriculturally based feedstocks, which limit their scale, unit economics, and sustainability. Alternatively, there are commercialized bioprocesses that address feedstock and scale limitations, such as algal and acetogenic approaches, but these bioprocesses face serious constraints on productivity, capital expenditures (CAPEX) costs, or control over the final molecular product. This approach addresses the limitations that other technologies face in feedstocks, productivity, and product tailoring, thus unlocking increased scale, improved economics, and meaningful sustainability.

This system bypasses the limitations of current gas fermentation approaches and retains the high product diversity that is possible in sugar-based fermentation by using an aerobic microorganism that uses H2 as an external reducing equivalent to fix CO₂ in the presence of oxygen. C. necator is able to use organic feedstock and gaseous feedstock, separately or in combination, and it is able to generate highly reduced hydrocarbon compounds at high yields. Glycerol has been used to de-repress hydrogenases in heterotrophic conditions. This system uses H2-enhanced mixotrophy with sugar in C. necator. Work with Clostridium ljungdahlii, Clostridium autoethanogemum, and cyanobacterial species demonstrate use with H2-enhanced sugar-based mixotrophy, which can be applied to C. necator. A defined media is used to maintain expression of hydrogenases during mixotrophic utilization of glycerol and H2.

### Example 2

Described herein is an exemplary embodiment in which bacteria can be cultured in at least one reactor chamber using the following protocol: (1) Mixotrophic growth, (2) switch to gas growth, (3) induce, and (4) switch to mixotrophic production. In some embodiments, the growth phase (e.g., steps (1)-(2)) can last between 0-30 days, and the production phase (e.g., steps (3)-(4)) can last between 0-14 days.

In some embodiments of any of the aspects, the gas concentrations can be in the following ranges: H₂ 1-99%, CO₂ 0.04-50%, O₂ 0.05-50%.

A variety of gas flow rates can be used. The gas flow rate can be measured in VVM, which stands for volume of gas sparged (e.g., in aerobic cultures) per unit volume of growth medium per minute: VVM can be calculated by dividing the measured gas flow rate (e.g., units: L/m, using a rotameter) by the volume (e.g., liters) of growth medium (e.g., including cultured cells). In one test, the gas flow rate was at from 0.1 to 3 throughout the run. In some embodiments of any of the aspects, the gas flow rate can range from 0.1 to 5 VVM.

During mixotrophic growth, the feedstock (e.g., an organic carbon source) supply rate can be set a specific rate. For example, during a specified time period during the production period, the mixotrophic feedstock supply rate can be from 0 to 50 g/L/hr. The specified time period can range from 1 minute to the entire run. In some embodiments, a bolus of organic carbon can be supplied at between 0-50 g/L. There can be time periods during the production period in which no feedstock or organic carbon is added.

The gas consumption rates (e.g., by the bacteria) can be as follows: H₂ 0-10,000 mmol/L/hr: CO₂ 0-5,000 mmol/L/hr: O₂ 0-5,000 mmol/L/hr. Gas consumption can be measured by analyzing the gas inlet into and gas outlet out of the at least one reactor chamber and then determining a mass balance of the gas.

The percent reduction in CO₂ during the mixotrophic production phase (e.g., step (4) above) can be a reduction from 100% CO₂ (e.g., in steps (1)-(3) above) to 0% CO₂ the mixotrophic production phase.

The specific growth rate was between 0-0.3 hr⁻¹ for this Example, which was faster than any other system for this level of titer. This maximum specific growth rate shows that the system performed unexpectedly well, with a maximum specific growth rate that was faster than gas or sugar only. The specific growth rate can be measured as cell per cell per hour or bioproduct per cell per hour. Cell number can be measured using standard techniques, e.g., by OD measurements, or serial dilution and plating. The bioproduct can be measured using standard techniques according to the specific bioproduct. For example, thin layer chromatography (TLC) can be used to detect bioproducts, such as TAGs.

Bioproducts can be produced using the system as described herein. For example, TAG production can from 2-26 hours. Using the process described in this example, Bioproduct (e.g., TAG) production continued to increase throughout the production phase, e.g., from 2-26 hours.

### Example 3

Described herein is an exemplary embodiment in which bacteria can be cultured in at least one reactor chamber using the following protocol: (1) gas growth, (2) induction, (3) gas production, and (4) switch to mixotrophic production.

In some embodiments of any of the aspects, the gas concentrations can be in the following ranges: H₂ 1-99%, CO₂ 0.04-15%, O₂ 0.05-20%

In this example, the gas flow rate was 0 to 3 vvm. In some embodiments of any of the aspects, the gas flow rate can range from 0.1 to 5 vvm

In this Example, the mixotrophic feedstock supply rate included a bolus of 20 grams of organic carbon material.

The specific growth rate for this example was 0-0.21 hr⁻¹. Bioproduct (e.g., TAG) production can occur from 0-7 days during the production phase.

1. A system for producing a bioproduct comprising:
at least one reactor chamber containing therein at least one solution selected from:
a) at least one growth solution comprising:
i) carbon dioxide (CO₂), hydrogen (H2), and oxygen (O₂); or
ii) an organic carbon source, hydrogen (H2), and oxygen (O₂), and optionally carbon dioxide (CO₂); and/or
b) at least one production solution comprising:
i) carbon dioxide (CO₂), hydrogen (H2), and oxygen (O₂);
ii) an organic carbon source, hydrogen (H2), and oxygen (O₂), and optionally carbon dioxide (CO₂); or
iii) an organic carbon source and oxygen (O₂); and
wherein the at least one reactor chamber contains therein:
c) at least one bacterium in the at least one growth solution and/or at least one production solution, wherein the at least one bacterium produces the bioproduct.

2. The system of claim 1, wherein the system comprises one reactor chamber.

3. The system of claim 1, wherein at least a portion of the growth solution can be removed from the at least one reactor chamber, and/or at least a portion of the production solution can be added to the at least one reactor chamber.

4. (canceled)

5. The system of claim 1, wherein the system comprises at least two reactor chambers.

6. The system of claim 1, wherein the at least one reactor chamber containing the at least one growth solution is a continuous fermentation reactor chamber, a gas fermentation reactor chamber, or a mixotrophic fermentation reactor chamber.

7-8. (canceled)

9. The system of claim 1, wherein the at least one reactor chamber containing the at least one production solution is a fed-batch fermentation reactor chamber.

10. The system of claim 1, wherein the at least one reactor chamber containing the least one production solution is:
a) a gas fermentation reactor chamber;
b) a gas and organic carbon (mixotrophic) fermentation reactor chamber; or
c) an organic carbon fermentation reactor chamber.

11. The system of claim 1, wherein the at least one reactor chamber containing the at least one growth solution or the at least one production solution emits no CO₂.

12. (canceled)

13. The system of claim 1, wherein the at least one reactor chamber containing the at least one production solution emits at most 1 molecule of CO₂ per molecule of acetyl-CoA.

14. (canceled)

15. The system of claim 1, wherein the at least one reactor chamber further comprises:
(a) an isolated gas volume above a surface of the at least one growth solution and/or at least one production solution within a headspace of the at least one reactor chamber, wherein the isolated gas volume comprises carbon dioxide (CO₂), hydrogen (H2), and/or oxygen (O₂); and/or
(b) a power source comprising a renewable source of energy, wherein the renewable source of energy comprises a solar cell, wind turbine, generator, battery, or grid power.

16-18. (canceled)

19. A system for producing a bioproduct comprising:
a) a primary reactor chamber with a at least one growth solution contained therein,
wherein the at least one growth solution comprises:
i) carbon dioxide (CO₂), hydrogen (H2), and oxygen (O₂), or
ii) an organic carbon source, hydrogen (H2), and oxygen (O₂), and optionally carbon dioxide (CO₂);
b) at least one secondary reactor chamber with a at least one production solution contained therein, wherein the at least one production solution comprises:
i) carbon dioxide (CO₂), hydrogen (H2), and oxygen (O₂);
ii) an organic carbon source, hydrogen (H2), and oxygen (O₂), and optionally carbon dioxide (CO₂); or
iii) an organic carbon source and oxygen (O₂); and
c) at least one bacterium in the at least one growth solution of the primary reactor chamber and/or at least one production solution of the secondary reactor chamber, wherein the at least one bacterium produces the bioproduct.

20-36. (canceled)

37. The system of claim 1, wherein the system comprises:
a) one growth solution and one production solution;
b) two growth solutions and one production solution; or
c) one growth solution and two production solutions.

38. The system of claim 1, wherein the system further comprises at least one inducer solution, wherein the at least one inducer solution:
(a) comprises a level of bioavailable nitrogen below a pre-determined threshold;
(b) comprises arabinose; and/or
(c) further comprises:
(i) carbon dioxide (CO₂), hydrogen (H2), and oxygen (O₂);
(ii) an organic carbon source, hydrogen (H2), and oxygen (O₂), and optionally carbon dioxide (CO₂); or
(iii) an organic carbon source and oxygen (O₂).

39-41. (canceled)

42. The system of claim 1, wherein the bacterium is:
(a) a chemolithotroph;
(b) a mixotroph capable of gas fermentation and organic carbon fermentation;
(c) a switchotroph capable of switching between gas fermentation and organic carbon fermentation;
(d) not a heterotroph; and/or
(e) Cupriavidus necator.

43-48. (canceled)

49. The system of claim 1, wherein the bacterium:
(a) naturally produces the bioproduct;
(b) is engineered to produce the bioproduct; and/or
(c) is capable of being induced to produce the bioproduct.

50-51. (canceled)

52. The system of claim 1, wherein the bioproduct is capable of being isolated, collected, or concentrated after the bacterium produces a pre-determined concentration of the bioproduct.

53. The system of claim 1, wherein the organic carbon source is selected from the group consisting of: glucose, glycerol, gluconate, acetate, fructose, decanoate, fatty acid, and glycerol gluconate.

54. (canceled)

55. The system of claim 1, wherein the at least one growth solution and/or at least one production solution comprises:
(a) cell culture medium;
(b) defined medium;
(c) minimal medium; and/or
(d) rich medium.

56-58. (canceled)

59. The system of claim 1, wherein the bioproduct is:
(a) selected from the group consisting of: polypeptide, glycoprotein, lipoprotein, lipid, monosaccharide, polysaccharide, nucleic acid, small molecule, or metabolite; and/or
(b) selected from the group consisting of: polyhydroxyalkanoate (PHA); sucrose; lipochitooligosaccharide; and triacylglyceride.

60. (canceled)

61. A method of a culturing a bacterium, the method comprising:
a) culturing the bacterium in at least one reactor chamber with at least one growth solution contained therein, wherein the at least one growth solution comprises:
i) carbon dioxide (CO₂), hydrogen (H2), and oxygen (O₂); or
ii) an organic carbon source, hydrogen (H2), and oxygen (O₂), and optionally carbon dioxide (CO₂);
b) adding at least one production solution to the at least one reactor chamber, wherein the at least one production solution comprises:
i) carbon dioxide (CO₂), hydrogen (H2), and oxygen (O₂);
ii) an organic carbon source, hydrogen (H2), and oxygen (O₂), and optionally carbon dioxide (CO₂); or
iii) an organic carbon source and oxygen (O₂); and
c) culturing the bacterium in the at least one production solution.

62-135. (canceled)
